(19) United States
(12) Patent Application Publication
(10) Pub. No.: US 2025/0001400 A1
(43) Pub. Date: Jan. 2, 2025

(54) MATERIAL AND METHOD FOR PERFORMING A SEPARATION BASED ON BORON CLUSTERS

(71) Applicant: MERCK PATENT GMBH, Darmstadt (DE)

(72) Inventors: Romas SKUDAS, Darmstadt (DE); Annika HOLZGREVE, Darmstadt (DE); Michael SCHULTE, Darmstadt (DE); Fabian KEPPNER, WUERZBURG (DE); Tanja KNUPLEZ, WUERZBURG (DE); Michael HAILMANN, WUERZBURG (DE); Maik FINZE, WUERZBURG (DE); Nikolai IGNATYEV, Darmstadt (DE)

(73) Assignee: MERCK PATENT GMBH, DARMSTADT (DE)

(21) Appl. No.: 18/708,273

(22) PCT Filed: Nov. 7, 2022
(86) PCT No.: PCT/EP2022/080959

§ 371 Date: May 8, 2024

(30) Foreign Application Priority Data
Nov. 8, 2021 (EP) 21206997.5

(51) Int. Cl. B01J 20/289; B01D 15/42; B01J 20/02; B01J 20/28; C07K 1/22

(52) U.S. Cl. CPC B01J 20/289; B01D 15/424; B01J 20/0248; B01J 20/28016; B01J 20/28033; C07K 1/22

(57) ABSTRACT

This invention relates to a new stationary phase carrying boron clusters. Target molecules can interact with this stationary phase depending on the cluster type and the substituents. The stationary phase is suitable for SPE and chromatographic separations.

This invention relates to a new stationary phase carrying boron clusters. Target molecules can interact with this stationary phase depending on the cluster type and the substituents. The stationary phase is suitable for SPE and chromatographic separations.

### BACKGROUND OF THE INVENTION

Boron clusters are polyhedral boron derivatives possessing unique properties. Among boron cluster types, closo-boranes (especially 10- and 12-vertex cages), closo-carboranes (especially 12-vertex cages), metallacarboranes, and selected nido-boranes are of particular interest. These compounds exhibit many advantageous properties in comparison to related organic counterparts, namely: unique chemical, electrochemical, and thermal stabilities e.g. of [closo-B₁₂H₁₂]²⁻, [closo-1-CB₁₁H₁₁]⁻, and closo-C₂B₁₀H₁₂ and metallabisdicarbollides [3,3′-M (1,2-C₂B₉H₁₁)₂]⁻ (M=Co³⁺, Fe³⁺, etc.). Boron clusters show both, electron-withdrawing and electron-donating properties depending on the cluster type and the substituents bonded to the carbon or boron atoms. A tunable molecular volume and geometry in combination with an adjustable hydrophilicity allow fine tuning of the properties of the materials containing such boron clusters.

Boron clusters are of interest for applications in biology and medicine, for example for boron neutron capture therapy (BNCT) for the treatment of cancer. The development of novel bioactive molecules and potential drugs containing boron clusters is another topic of actual research in this emerging area.

It has been reported that many polyhedral boranes are basically nontoxic due to their inertness to biochemical processes (Plešek, J. Potential Applications of the Boron Cluster Compounds. Chem. Rev. 1992, 92, 269-278.). For the boron enrichment in tumor cells, icosahedral boron clusters are highly promising due to their high number of boron atoms per molecule. In this context, current research is focused on the use of icosahedral closo-boron clusters, for instance carba-closo-dodecaboranes, carba-closo-dodecaborate anions or closo-dodecaborate anions.

The weakly acidic CH unit(s) of carba-closo-dodecaboranes and carba-closo-dodecaborate anions can be deprotonated and thus provide the possibility for a comparably easy substitution chemistry and further derivatization (Grimes, R. N. Carboranes; 3 ed.; Academic Press/Elsevier Inc.: London, U K, 2016.).

A series of adenosine derivatives modified either with boron clusters or substituted phenyls were synthesized and their physicochemical and biological properties were compared. Noticeably, a positive effect of boron clusters was detected. This result is an example, which indicates that boron clusters are useful tools for the tuning of properties of biomolecules. It provides an instrument for the modification of nucleoside structures or other biomolecules with the aim of the development of novel drugs.

Due to their specific properties, carborane clusters are of particular interest as substituents and ligands in supramolecular chemistry. Carba-closo-dodceaboranes and related icosahedral boron clusters, in general, are applied as bulky pharmacophores, e.g. for the replacement of various hydrophobic units in biologically active molecules. The introduction of 12-vertex carboranes into biologically active molecules often increases their in vivo stability and bioavailability and enhances interactions between pharmaceuticals and receptors due to the hydrophobic properties of these boron clusters. Carboranes are very stable towards degradation by enzymes. Consequently, the usage of boron clusters as components of new pharmaceuticals is growing in the last years.

The interaction of selected boron clusters and their derivatives with serum albumin, the most abundant protein in mammalian blood, has been reported (Goszczyński, T. M.; Fink, K.; Kowalski, K.; Leśnikowski, Z. J.; Boratyński, J. Interactions of Boron Clusters and their Derivatives with Serum Albumin. Sci. Rep. 2017, 7, 9800.). The results of this study demonstrate that metallacarboranes strongly interact with albumin. The observed strength of boron cluster interactions with albumin follows the order: metallacarboranes [M(C₂B₉H₁₁)₂]⁻>carboranes (C₂B₁₀H₁₂)>>dodecaborate anion [B₁₂H₁₂]²⁻. Metallacarboranes interact specifically with the binding cavity of albumin and non-specifically with the protein surface. The authors highlight the importance of the findings for the development of new bioactive compounds that contain boron clusters.

The examples described above demonstrate the versatility for the application of boron clusters in various fields.

However, the application of boron clusters in chromatography materials for the separation and/or purification of biomolecules is still unknown and bears a potential for high selectivity for selected biopharmaceutical molecules and the purification thereof.

### BRIEF DESCRIPTION OF THE INVENTION

The present invention is thus directed to the application of boron clusters in stationary phases for the separation and/or purification of a target molecule, in particular in solid phase extraction (SPE) and chromatographic separation. Those separation materials bear a potential for high selectivity for target molecules, e.g. biomolecules and the purification thereof.

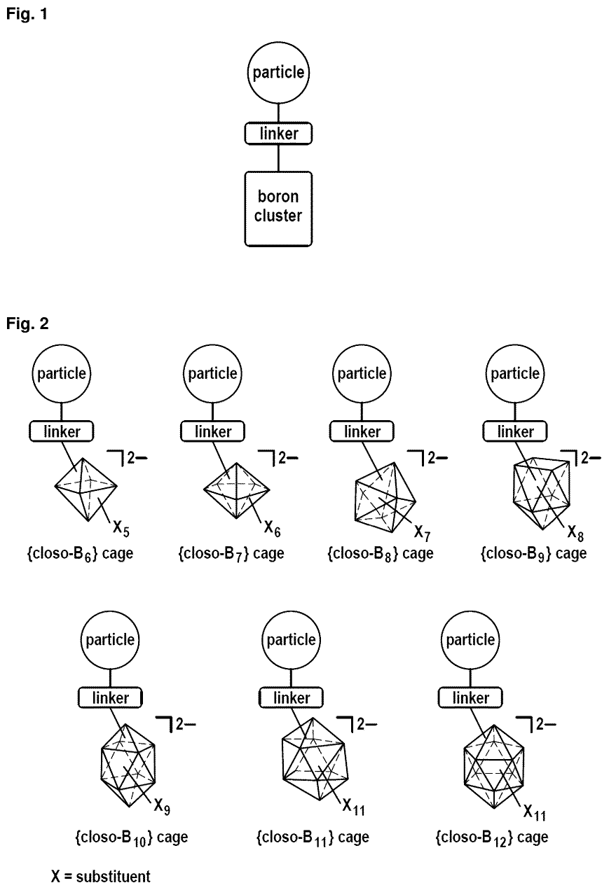

The boron clusters that can vary in cluster size, geometry, charge, atom composition (all-boron or cages with heteroatoms), and substituents bonded to the cluster atoms are attached to a particle (base material) preferably via a linker (FIG. 1).

Furthermore the present invention is directed to a stationary phase comprising a boron cluster, a separation device comprising the stationary phase and a process for separation of a target molecule from at least one other compound using the stationary phase or the separation device.

### DETAILED DESCRIPTION OF THE INVENTION

The present invention refers to a stationary phase comprising a base material and at least one boron cluster, wherein the boron cluster contains only main group elements of the periodic table.

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Unless defined otherwise, all technical and scientific terms used herein have the same meaning as commonly understood by one of ordinary skill in the art to which this invention is related. The following terms are defined for purposes of the invention as described herein.

According to the invention boron clusters are polyhedral boron derivatives (Beckett, M. A.; Brellochs, B.; Chizhevsky, I. T.; Damhus, T.; Hellwich, K.-H.; Kennedy, J. D.; Laitinen, R.; Powell, W. H.; Rabinovich, D.; Viñas, C.; Yerin, A. Nomenclature for boranes and related species (IUPAC Recommendations 2019). Pure Appl. Chem. 2020, 92, 355-381.). Boron clusters an their nomenclature according to the present invention are published by Beckett et al. 2020. In particular boron clusters include all boron closo-cages {closo-Bₙ} and other geometries such as nido-, arachno-, and hypho-clusters. The boron clusters are connected, preferably via a linker, to a suitable base material, indicated by “particle” in FIGS. 2 to 7. The boron clusters according to the present invention can be uncharged or charged, preferably the boron cluster has a charge of −1 or −2. The charge of a boron cluster is typically adjusted by variation of the cluster type, by the introduction of heteroatoms (main group elements or transition metals) into the boron cage, or by the introduction of charged substituents at the boron cluster, e.g. NH₃⁺. Substitution of boron clusters is indicated by X₅ to X₁₁ and X₁₇ in FIGS. 2 to 7.

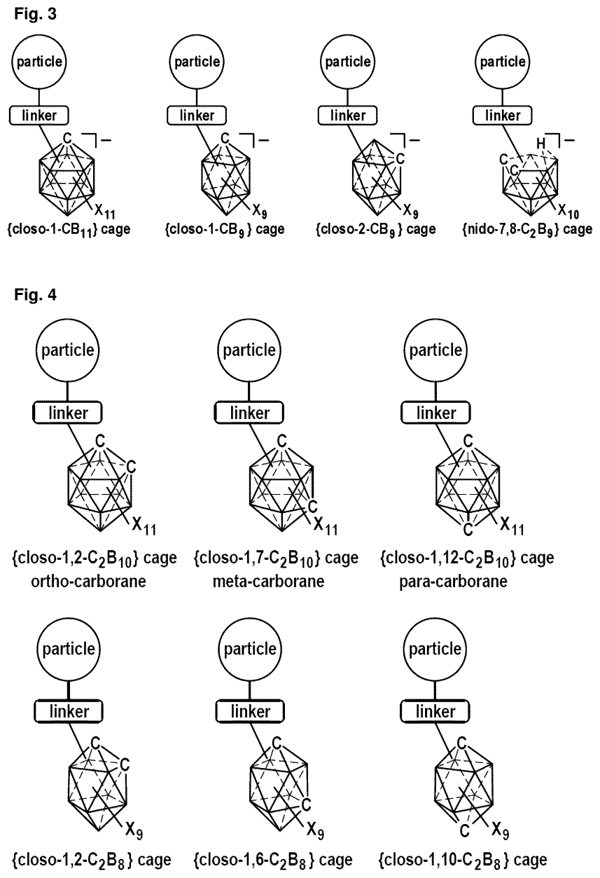

One or more boron atoms can be replaced by heteroatoms. Replacement of one or more boron atoms by heteroatoms in the boron clusters leads to other boron cage-based end groups with tunable properties. Preferably, the heteroatom is carbon (carborane) as exemplified by monocarbon boron clusters shown in FIG. 3 and dicarbon boron clusters depicted in FIG. 4 (Grimes, R. N. Carboranes; 3 ed.; Academic Press/Elsevier Inc.: London, U K, 2016.).

In addition to boron clusters that contain one or two carbon atoms, related heteroatom clusters with other main group atoms are part of the present invention. These heteroatoms include, for example, tin, lead, silicon, and phosphorus. The position of the heteroatom depends on the cluster type (size & geometry) and the type of heteroatom and the number of heteroatoms. Typical positions are shown in FIG. 3 and FIG. 4. However, other positions are possible.

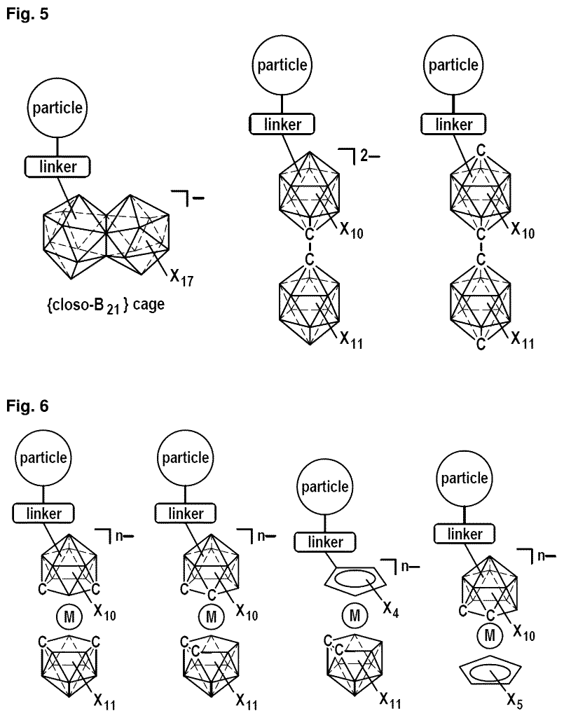

conjuncto-Boron clusters represent a further class of boron clusters according to the invention. These conjuncto-clusters include all-boron cages such as the {closo-B₂₁} cluster and clusters with heteroatoms, e.g. carbon, as exemplified in FIG. 5.

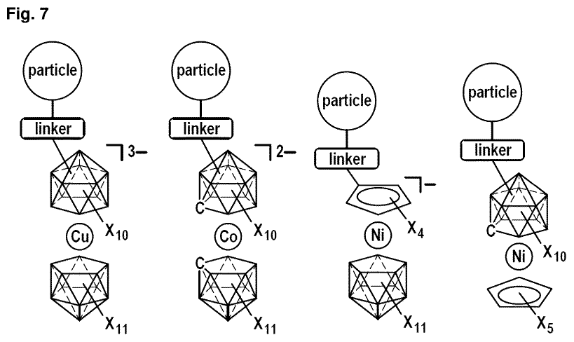

Metallacarboranes are a further type of polyhedral boron clusters. These clusters can differ in (i) (transition) metal atom(s), (ii) additional heteroatoms (=non-boron atoms), (iii) cluster size (number of cluster atoms), (iv) charge, (v) further ligands at the metal atoms (e.g. cyclopentadienyl, Cp), and (vi) substituents bonded to the cluster. In FIG. 6 examples for metallacarboranes attached to a particle are depicted and FIG. 7 shows more specific examples based on the metals Cu, Co, and Ni.

Among boron cluster types, closo-boranes (especially 12-vertex cages), closo-carboranes (especially 12-vertex cages), and metallacarboranes are of particular interest. These compounds exhibit many advantageous properties in comparison to their organic counterparts, namely: unique chemical, electrochemical, and thermal stabilities e.g. of [closo-B₁₂H₁₂]²⁻, [closo-1-CB₁₁H₁₁], and closo-C₂B₁₀H₁₂ and metallabisdicarbollides [3,3′-M(1,2-C₂B₉H₁₁)₂]⁻ (M=Co³⁺, Fe³⁺, etc.). Boron clusters show both, electron-withdrawing and electron-donating properties depending on the cluster type and the substituents bonded to the carbon or boron atoms.

One embodiment of the present invention is a stationary phase comprising a base material and at least one boron cluster.

For the avoidance of doubt, the term boron cluster refers to separate polyhedral boron molecules as described above and does not incorporate boronpolymers.

In a preferred embodiment, the boron cluster contains only main group elements of the periodic table. Boron clusters that contain only main group elements exclude boron clusters which comprise at least one transition metal element, e.g. Cu, Co or Ni. Main group elements include the elements of group 1 and 2 (s-block), and groups 13 to 18 (p-block) of the periodic table.

The term “boron cluster contains only main group elements of the periodic table” can be replaced by “boron cluster only contains main group elements of the periodic table” or “boron cluster consists of main group elements of the periodic table”.

Stationary phases comprising a base material and at least one boron cluster show very good separation properties, in particular with binding proteins as shown in Example 2. Stationary phases comprising a boron cluster wherein the boron cluster contains only main group elements of the periodic table have advantageous separation properties compared to stationary phase wherein the boron cluster contains at least one transition element, e.g. Cu, Co or Ni. It could be shown that the elution of bound target molecules is comparably better with boron cluster only containing main group elements of the periodic table. Examples 2.1 to 2.4 show separation experiments using separation material with boron cluster only containing main group elements of the periodic table. The concentration of bound protein in Run 1 to Run 3 is similar in throughout the different separation materials, in general. Example 2.5 shows a separation experiment using a separation material with a boron cluster containing cobalt (a derivative of the parent COSAN cluster [3,3′-Co (1,2-C₂B₉H₁₁)₂]⁻), which is a transition element. It can be seen that the concentration of bound protein decreases by more than 50% from Run 1 to Run 2. This indicates that the interaction of target molecules, in particular proteins, with boron clusters only containing main group elements of the periodic table are better controllable which is beneficial for separation of target molecules.

A similar trend can be seen for separation materials with boron clusters having a charge of −1 or −2, preferably −2.

In a further embodiment the boron cluster has a closo, nido, arachno, hypho, hypercloso or conjuncto cluster type. Preferably the cluster type is selected from the group consisting of closo, nido, arachno, hypho, hypercloso, conjuncto, more preferably closo, nido or conjuncto, most preferably closo. In a further embodiment the boron cluster has a conjuncto cluster type. In a further embodiment the boron cluster is a {closo-B₂₁} cluster.

In a further embodiment the boron cluster has a cluster size of 6, 7, 8, 9, 10, 11 or 12 atoms per cluster. In a further embodiment, the boron cluster has a cluster size of not more than 12 atoms.

In a further embodiment the boron cluster contains 1, 2 or more atoms that are not a boron atom. Preferably, the at least one atom that is not a boron atom is selected from the group consisting of carbon, sulfur, nitrogen, germanium, tin, lead and phosphorus, more preferably carbon.

In a further embodiment the boron cluster is uncharged or has a charge of −1 or −2, preferably the boron cluster has a charge of −1 or −2, more preferably the boron cluster has a charge of −2.

In a further embodiment the boron cluster is a metallacarborane. Preferably, the at least one transition metal atom is selected from the group consisting of Cu, Co, Fe and Ni.

Boron cluster are optionally substituted by one or more covalently bonded substituents. Preferably, those substituents are further functional groups according to the definition below.

In a further embodiment one or more hydrogen atoms of the boron cluster are replaced by a covalently bonded substituent. Preferably, the covalently bonded substituent is selected from a list consisting of
(1) halogen,
(2) pseudohalogen,
(3) —NR¹R²,
   wherein R¹ and R² independently of one another represent H, alkyl, aryl, heteroaryl or heteroalkyl,
(4) —N⁺R¹R²R³,
   wherein R¹, R² and R³ independently of one another represent H, alkyl, aryl, heteroaryl or heteroalkyl,
(5) hydroxy,
(6) —OR⁴,
   wherein R⁴ represents alkyl, aryl, heteroaryl or heteroalkyl,
(7) —O⁺R⁴R⁵ or —S⁺R⁴R⁵,
   wherein R⁴ and R⁵ independently of one another represent alkyl, aryl, heteroaryl or heteroalkyl,
(8) alkyl,
(9) cycloalkyl,
(10) (per) fluoroalkyl,
(11) alkenyl,
(12) alkynyl,
(13) aryl,
(14) heteroaryl,
(15) —C(═O) OR⁶,
   wherein R⁶ represents H, alkyl, aryl, heteroalkyl, heteroaryl or boranyl,
(16) —C(O) R⁶,
   wherein R⁶ represents H, alkyl, aryl, heteroalkyl, heteroaryl or boranyl,
(17) acid derivatives such as acid amides, imides, ureas, thioureas, guanidine and its derivatives,
(18) —SR⁷,
   wherein R⁷ represents H, alkyl or aryl,
(19) —SSR⁸,
   wherein R⁸ represents alkyl, aryl or boranyl,
(20) —SeR9,
   wherein R⁹ represents alkyl, aryl or heteroaryl,
(21) —TeR⁹,
   wherein R⁹ represents alkyl, aryl or heteroaryl,
(22) —C(S) R¹⁰,
   wherein R¹⁰ represents alkyl or aryl,
(23) —BR¹¹R¹²,
   wherein R¹¹ and R¹² independently of one another represent alkyl, aryl or heteroaryl,
(24) —B⁻R¹³R¹⁴R¹⁵
   wherein R¹³, R¹⁴ and R¹⁵ independently of one another represent H, alkyl, aryl, heteroaryl or heteroalkyl,
(25) guanidinium,
(26) sugar derivatives
(27) —P(O) (OR¹⁶) R¹⁷, and
   wherein R¹⁶ and R¹⁷ independently of one another represent H, alkyl, aryl, heteroaryl or heteroalkyl; R¹⁷ can be a fluorinated alkyl, aryl or heteroaryl
(28) —SiR¹⁸R¹⁹R²⁰ and —Ge¹⁸R¹⁹R²⁰
   wherein R¹⁸, R¹⁹ and R²⁰ independently of one another represent H, alkyl, aryl, heteroaryl, heteroalkyl, hydroxy or alkoxy

More preferably, the covalently bonded substituent is selected from a list consisting of halogen, hydroxy, cyano, carboxy, amino and —N⁺R¹R²R³, wherein R¹, R² and R³ independently of one another represent H, alkyl, aryl, heteroaryl or heteroalkyl.

In one embodiment the at least one boron cluster is selected from the group consisting of boron clusters of Table 1.

TABLE 1
Selected boron clusters with position of the attachment to
linker or base material (particle)
{closo-B₁₂X₁₁}

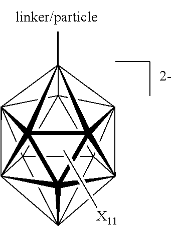

{closo-B₁₀X₉}

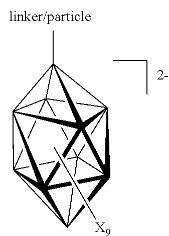

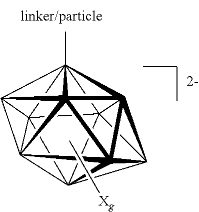

{closo-B₆X₅}

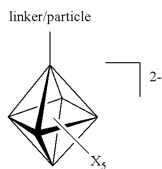

{closo-1-CB₁₁X₁₁}

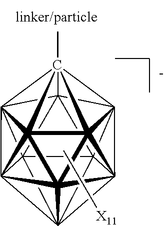

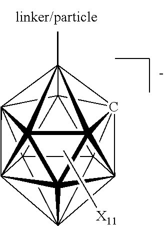

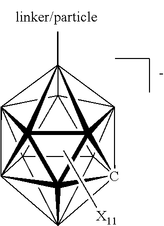

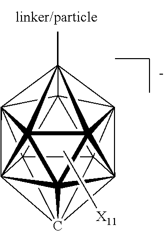

{closo-1-CB₉X₉}

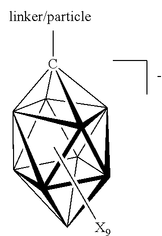

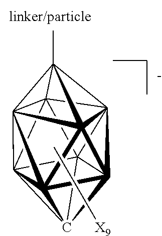

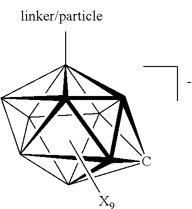

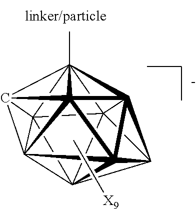

{closo-1,2-C₂B₁₀X₁₁}

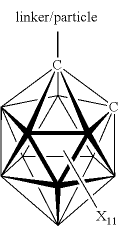

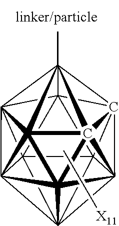

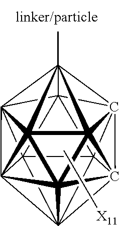

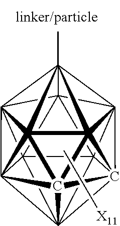

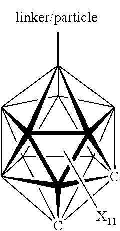

{closo-1,7-C₂B₁₀X₁₁}

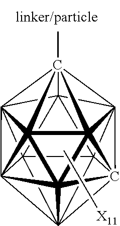

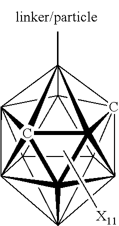

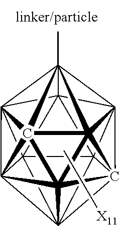

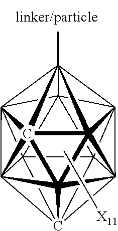

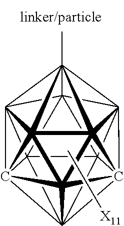

{closo-1,12-C₂B₁₀X₁₁} (left) and {closo-1,7-C₂B₁₀X₁₁} (right)

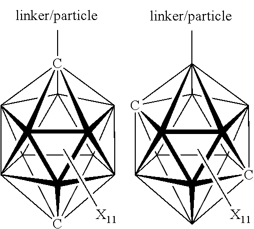

{nido-7,8-C₂B₉X₁₀}

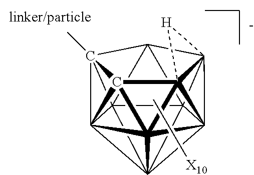

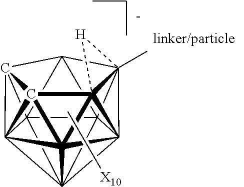

{nido-7,9-C₂B₉X₁₀}

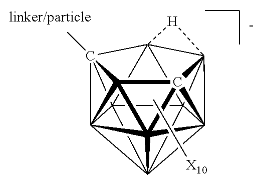

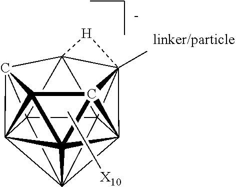

{Co(C₂B₉X₁₁)(C₂B₉X₁₀)}

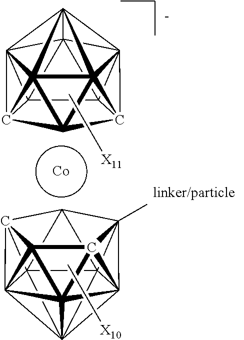

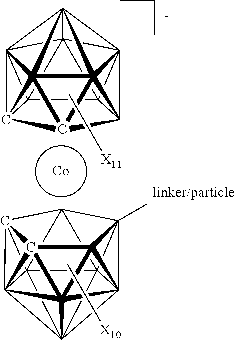

{Fe(C₂B₉X₁₁)(C₂B₉X₁₀)}

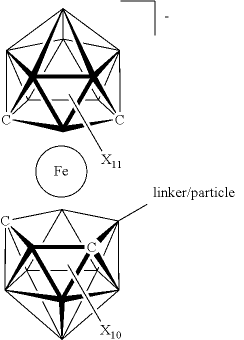

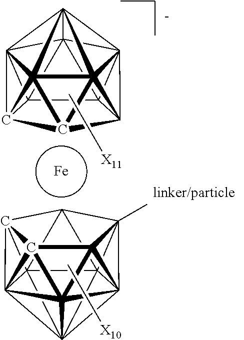

X₅, X₉, X₁₀ and X₁₁ represent one or more covalently bonded substituents as defined above

In one embodiment the at least one boron cluster is selected from the group consisting of the parent boron clusters of Table 2, which solely have hydrogen substituents bonded to the cluster. One of these hydrogen substituents is formally replaced by a linker to allow attachment to a particle.

TABLE 2
Selected parent hydrogen-substituted boron clusters
[closo-B₁₂H₁₂]⁻

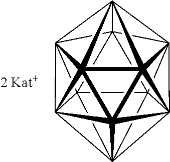

[closo-B₁₀H₁₀]⁻

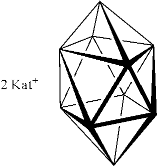

[nido-7,8-C₂B₉H₁₂]⁻

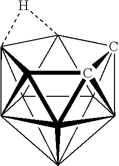

closo-1,2-C₂B₁₀H₁₂

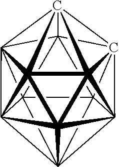

closo-1,7-C₂B₁₀H₁₂

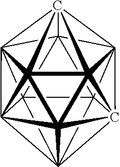

closo-1,12-C₂B₁₀H₁₂

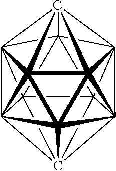

[closo-CB₁₁H₁₂]⁻

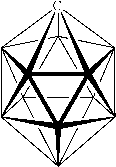

[12-I-closo-CB₁₁H₁₁]⁻

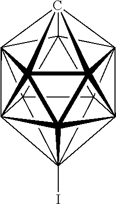

[3,3′-Co(1,2-C₂B₉H₁₁)₂]⁻

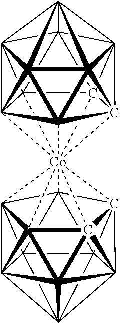

Boron cluster of the present invention can bind target molecules directly. Optionally, functional groups with particular binding properties are attached to the boron cluster.

Thus, the inventive materials and stationary phases are especially flexible and tunable to adapt to different separation processes.

As used herein the term “target molecule” refers to any molecule, substance or compound that shall be isolated, separated or purified from one or more other components, e.g. impurities, in a sample. In the production and/or purification process the target molecule is typically present in a liquid. The liquid might be water, a buffer, a non-aqueous organic solvent like ethanol, acetonitrile, heptane or any mixture of the named liquids. Beside the target molecule said liquid may comprise one or more impurities. The liquid may also be called sample. The composition of the liquid may change during production and/or purification depending on the process steps that are performed. After a chromatographic step the liquid typically comprises other solvents than before because of the eluent used in the chromatographic step. Examples of target molecules are low molecular weight molecules like drugs having a molecular weight around or below 2000 g/mol. A target molecule may also be a high molecular weight compound like a protein, e.g. an antibody. Further examples of target molecules are biopolymers from natural sources, biopolymers from recombinant sources, proteins and peptides, monoclonal and polyclonal antibodies, viruses like lentivirus, adenovirus, adeno associated virus, measles virus, virus like particles, exosomes, host cell proteins, ADCs, alkaloids, lipids like diglycerides or triglycerides, carbohydrates, nucleic acids like mRNA or plasmid DNA. A preferred target molecule is a protein, e.g. an antibody.

A “protein” is herein defined as a polymer of amino acids linked to each other by peptide bonds to form a polypeptide. Preferably the chain length of which is sufficient to produce at least a detectable tertiary structure. Proteins are naturally occurring or non-naturally occurring, synthetic, or semisynthetic. The term “protein” is understood to also cover peptides, oligopeptides, polypeptides and any therapeutic protein as defined below.

The term “therapeutic proteins” as used herein refers to any protein or polypeptide that is administered to a subject with the aim of treating or preventing a disease or medical condition. In particular, the subject may be a mammal or a human. Therapeutic proteins are administered for different purposes, such as replacing a protein that is deficient or abnormal, augmenting an existing pathway, providing a novel function or activity, interfering with a molecule or organism and delivering other compounds or proteins, such as a radionuclide, cytotoxic drug, or effector proteins. Therapeutic proteins encompass antibodies, antibody-based drugs, Fc fusion proteins, anticoagulants, blood factors, bone morphogenetic proteins, engineered protein scaffolds, enzymes, growth factors, hormones, interferons, interleukins, antibody drug conjugates (ADCs) and thrombolytics. Therapeutic proteins can be naturally occurring proteins or recombinant proteins. Their sequence can be natural or engineered.

The term “antibody” refers to a protein which has the ability to specifically bind to an antigen. “Antibody” or “IgG” further refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

An exemplary immunoglobulin (antibody) structural unit is composed of two pairs of polypeptide chains, each pair having one “light” (about 25 kD) and one “heavy” chain (about 50-70 kD), said chains being stabilized, for example, by interchain disulfide bonds. The N-terminus of each chain defines a variable region of about 100 to 110 or more amino acids primarily responsible for antigen recognition. The terms variable light chain (V L) and variable heavy chain (V H) refer to these light and heavy chains respectively.

Antibodies may be monoclonal or polyclonal and may exist in monomeric or polymeric form, for example, IgM antibodies which exist in pentameric form and/or IgA antibodies which exist in monomeric, dimeric or multimeric form. Antibodies may also include multispecific antibodies (e.g., bispecific antibodies), and antibody fragments so long as they retain, or are modified to comprise, a ligand-specific binding domain. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. When produced recombinantly, fragments may be expressed alone or as part of a larger protein called a fusion protein. Exemplary fragments include Fab, Fab′, F(ab′)2, Fc and/or Fv fragments. Exemplary fusion proteins include Fc fusion proteins. According to the present invention fusion proteins are also encompassed by the term “antibody”.

In some embodiments, an antibody is an Fc region containing protein, e.g., an immunoglobulin.

As used herein, and unless stated otherwise, the term “sample” refers to any composition or mixture that contains a target molecule. Samples may be derived from biological or other sources. Biological sources include eukaryotic sources like animals or humans. The sample may also include diluents, buffers, detergents, and contaminating species and the like that are found mixed with the target molecule.

The term “impurity” or “contaminant” as used herein, refers to any foreign or objectionable molecule, including a biological macromolecule such as DNA, RNA, one or more host cell proteins, nucleic acids, endotoxins, lipids, impurities of synthetic origin and one or more additives which may be present in a sample containing the target molecule that is being separated from one or more of the foreign or objectionable molecules.

The terms “purifying,” “separating,” or “isolating,” as used interchangeably herein, refer to increasing the degree of purity of a target molecule by separating it from a composition or sample comprising the target molecule and one or more other components, e.g. impurities. Typically, the degree of purity of the target molecule is increased by removing (completely or partially) at least one impurity from the composition.

The term “chromatography” refers to any kind of technique which separates an analyte of interest (e.g. a target molecule) from other molecules present in a mixture. Usually, the target molecule is separated from other molecules as a result of differences in rates at which the individual molecules of the mixture migrate through a stationary phase under the influence of a moving phase, or in bind and elute processes. Examples for chromatographic separation processes are reversed phase chromatography, ion exchange chromatography, size exclusion chromatography, affinity chromatography, hydrophobic interaction chromatography and mixed mode chromatography. The chromatography process of the present invention is based on interaction with boron clusters as well as optionally additionally on one or more of the other separation processes mentioned above.

A “buffer” is a solution that resists changes in pH by the action of its acid-base conjugate components. Various buffers which are typically employed depending, for example, on the desired pH of the buffer are described in Buffers. A Guide for the Preparation and Use of Buffers in Biological Systems, Gueffroy, D., ed. Calbiochem Corporation (1975). Non-limiting examples of buffers include MES, MOPS, MOPSO, Tris, HEPES, phosphate, acetate, citrate, succinate, glycine and ammonium buffers, as well as combinations of these. An aqueous buffer is a buffer whose solvent comprises more than 90%, preferably 100% water.

The term “stationary phase” refers to any kind of sorbent, matrix, resin or solid phase which in a separation process separates a target molecule from other molecules present in a mixture. Usually, the target molecule is separated from other molecules as a result of differences in rates at which the individual molecules of the mixture bind to the stationary phase and/or migrate through the stationary phase under the influence of a moving phase. Stationary phases are typically put in columns or cartridges. The stationary phase according to the present invention comprises a base material and at least one boron cluster.

A “functional group” is a ligand that is attached to the base material of a stationary phase and that determines the binding properties of the stationary phase. Examples of “functional groups” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). The stationary phases according to the present invention comprise at least one boron cluster as functional group. They might comprise additionally one or more other functional groups as listed above. Those other functional groups are typically either attached to the boron cluster or attached to the base material independent of the boron cluster. Often, one functional group or one boron cluster has more than one binding property.

When “loading” a chromatography column in bind and elute mode, a buffer is used to load the sample or composition comprising the target molecule and one or more impurities onto a chromatography column. The buffer has a conductivity and/or pH such that the target molecule is bound to the stationary phase while ideally all the impurities are not bound and flow through the column. The separation of the bound target molecule from the one or more impurities can additionally be done with a change of conductivity and/or pH such that the target molecule is washed or eluted before or after one or more impurities.

Typically the buffer in which the sample is loaded on the stationary phase is called loading buffer or sample buffer.

When “loading” a chromatography column to “flow through” a target molecule, a buffer is used to load the sample or composition comprising the target molecule and one or more impurities onto a chromatography column. The buffer has a conductivity and/or pH such that the target molecule is not bound to the stationary phase and flows through the column while ideally all the impurities are bound the column.

The term “equilibrating” refers to the use of a buffer to equilibrate the stationary phase prior to loading the target molecule. Typically, the loading buffer is used for equilibrating.

By “wash” or “washing” a stationary phase is meant passing an appropriate liquid, e.g. a buffer through or over the stationary phase. Typically washing is used to remove weakly bound contaminants from the stationary phase in bind/elute mode prior to eluting the target molecule or to remove non-bound or weakly bound target molecule after loading.

In this case, typically, the wash buffer and the loading buffer are the same. In case a virus inactivation buffer is used, it is used to inactivate certain present virus prior to eluting the target molecule. In this case, typically, the virus inactivation buffer differs from loading buffer since it may contain detergent/detergents or have different properties (pH/conductivity/salts and their amounts).

Washing can also be used to remove contaminants from the stationary phase after the elution of the target molecule. This is done by passing an appropriate liquid, e.g. a buffer through or over the stationary phase after the elution of the target molecule. In this case, typically, the washing buffer differs from loading buffer. It may contain detergent/detergents or have different properties (pH/conductivity/salts and their amounts). The washing buffer can for example be an acidic buffer.

To “elute” a molecule (e.g. the target molecule or an impurity) from a stationary phase is meant to remove the molecule therefrom. Elution may take place directly in flow though mode when the target molecule is eluted with the solvent front of the loading buffer or by altering the solution conditions such that a buffer different from the loading buffer competes with the molecule of interest for the ligand sites on the stationary phase. A non-limiting example is to elute a molecule from an ion exchange resin by altering the ionic strength of the buffer surrounding the ion exchange material such that the buffer competes with the molecule for the charged sites on the ion exchange material.

The terms “flow-through process,” “flow-through mode,” and “flow-through operation,” as used interchangeably herein, refer to a separation technique in which at least one target molecule contained in a sample along with one or more impurities is intended to flow through a chromatography stationary phase, which usually binds the one or more impurities, where the target molecule usually does not bind (i.e., flows through) and is eluted from the stationary phase with the loading buffer.

The terms “bind and elute mode” and “bind and elute process,” as used herein, refer to a separation technique in which at least one target molecule contained in a sample binds to a suitable stationary phase and is subsequently eluted with a buffer different from the loading buffer.

Solid-phase extraction (SPE) is a sample preparation method where the compounds that are dissolved or suspended in a liquid mixture are separated from other compounds depending on their physical and chemical properties. The result is that either the target molecule or undesired impurities in the sample are retained on the stationary phase. The portion that passes through the stationary phase is collected or discarded, depending on whether it contains the target molecule or undesired impurities. If the portion retained on the stationary phase includes the target molecules, they can then be removed from the stationary phase for collection in an additional step, in which the stationary phase is rinsed with an appropriate eluent.

According to the present invention, particle size is determined by laser-diffraction, preferably with Malvern Master Sizer and pore size is determined by inverse SEC.

An electron withdrawing group includes atoms or groups of atoms that have electron-withdrawing inductive and/or mesomeric effects and which are typically more electronegative as hydrogen. Exemplary electron withdrawing groups are I, Br, Cl, F, CO₂H, NO₂, CN.

An electron donating group includes atoms or groups of atoms that have electron-donating inductive and/or mesomeric effects. Exemplary electron donating groups are —OH, O-alkyl, —NH₂, —NHalkyl, —N(alkyl)₂.

An alkyl or an alkyl group denotes a straight-chain or branched alkyl group typically having 1 to 20 C atoms or having the number of C atoms as indicated, for example methyl, ethyl, isopropyl, propyl, butyl, sec-butyl or tert-butyl, furthermore pentyl, 1-, 2- or 3-methylbutyl, 1,1-, 1,2- or 2,2-dimethylpropyl, 1-ethylpropyl, n-hexyl, n-heptyl, n-octyl, n-nonyl, n-decyl, n-undecyl or n-dodecyl, n-tridecyl, n-tetracecyl, n-pentadecyl, n-hexadecyl, n-heptadecyl, n-octadecyl, n-nonadecyl or n-eicosyl, preferably the alkyl group has 1 to 10 C atoms. C₁-C₄ alkyl, C₁-C₆ alkyl, C₁-C₈ alkyl, C₁-C₁₀ alkyl and C₁-C₁₅ alkyl denotes a straight-chain or branched alkyl group having the number of C atoms as indicated.

An alkoxy or alkoxy group in the context of the invention is a straight-chain or branched alkoxy radical typically having 1 to 20 C atoms or having the number of C atoms as indicated, e.g. a C₁-C₄ alkoxy is a straight-chain or branched alkoxy radical having 1 to 4 carbon atoms. Preferred examples include: methoxy, ethoxy, n-propoxy, iso-propoxy, n-butoxy, iso-butoxy, sec-butoxy and tert-butoxy.

A cycloalkyl or cycloalkyl group denotes a saturated hydrocarbon ring which contains 3, 4, 5, 6, 7, 8 or more carbon atoms or having the number of carbon atoms as indicated. Said cycloalkyl group is for example, a monocyclic hydrocarbon ring, e.g. a cyclopropyl, cyclobutyl, cyclopentyl, cyclohexyl, cycloheptyl or cyclooctyl group, a bicyclic hydrocarbon ring, e.g. a bicyclo[4.2.0] octyl or octahydropentalenyl, or a bridged or caged saturated ring groups such as norborane or adamantyl, and cubane, preferably cyclohexyl and adamantyl.

An heteroalkyl or heteroalkyl group denotes a straight-chain or branched alkyl group as defined above wherein the alkyl group contains one or more different heteroatoms from the series N, O and S.

An (per) fluoroalkyl, (per) fluoroalkenyl or (per) fluoroaryl group denotes aliphatic and/or aromatic groups with fluorine substituents. In perfluorinated groups all hydrogen atoms are replaced by fluorine. In partially fluorinated groups one, two, or more hydrogen atoms are replaced by fluorine. Examples are perfluorinated groups such as —CF₃, —C₂F₅, —C₆F₅, —CF=CF₂. Examples for partially fluorinated groups are —CH₂CF₃, —C₆H₃(CF₃)₂.

An alkenyl or alkenyl group denotes a straight-chain or branched alkenyl typically having 2 to 20 C atoms or having the number of C atoms as indicated, where, in addition, a plurality of double bonds may be present, is, for example, allyl, 2- or 3-butenyl, iso-butenyl, sec-butenyl, furthermore 4-pentenyl, iso-pentenyl, hexenyl, heptenyl, octenyl, —C₉H₁₇, —C₁₀H₁₉ to —C₂₀H₃₉; preferably allyl, 2- or 3-butenyl, iso-butenyl, sec-butenyl.

An alkynyl or alkynyl group denotes a straight-chain or branched alkynyl radical typically having 2 to 20 C atoms or having the number of C atoms as indicated and at least one triple bond. Those alkynyl groups are a straight-chain or branched alkynyl radical having 2 to 4 carbon atoms [(C₂-C₄)-alkynyl], e.g. ethinyl, prop-1-yn-1-yl, prop-2-yn-1-yl (propargyl), but-1-yn-1-yl, but-2-yn-1-yl, but-3-yn-1-yl and but-3-yn-2-yl.

An aryl or an aryl group denotes an aryl group typically having 6, 7, 8, 9, 10, 11, 12 or more C atoms or having the number of C atoms as indicated, for example phenyl, naphthyl or anthracenyl. C₆-C₁₀ aryl or C₆-C₁₈ aryl denotes an aryl group having the number of C atoms as indicated. The aryl group may be unsubstituted or substituted, for example by halogen, NH₂, NAlK₂, NHAlkyl, NO₂, CN, SO₃H or OAlkyl. The substitution may take place once or a number of times by the substituents indicated, preferably once.

An heteroaryl or heteroaryl group denotes a monovalent, monocyclic, bicyclic or tricyclic aromatic ring having 5, 6, 8, 9, 10, 11, 12, 13 or 14 ring atoms (a “5 to 14 membered heteroaryl” group), particularly 5, 6, 9 or 10 ring atoms, or having the number of C atoms as indicated, which contains at least one ring heteroatom and optionally one, two or three further ring heteroatoms from the series: N, O and/or S, and which is bound via a ring carbon atom or optionally via a ring nitrogen atom (if allowed by valency). Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl. In general, and unless otherwise mentioned, the heteroaryl or heteroarylene groups include all possible isomeric forms thereof, e.g.: tautomers and positional isomers with respect to the point of linkage to the rest of the molecule. Preferred heteroaryl groups are imidazolyl, pyridyl, furanyl, thienyl, substituted pyridyl and morpholyl.

A halogen denotes a moiety selected from the list comprising fluorine, chlorine, bromine, or iodine. Pseudohalogen or a pseudohalogen groups denote polyatomic analogues of halogens, e.g. a moiety such as cyano, isocyano, thiocyanato, cyanato or isocyanato.

A sugar derivative denotes a carbohydrate, in particular a monosaccharide or disaccharide, e.g. glucose, fructose, galactose, sucrose or lactose.

The base material is the material the boron cluster is attached to. According to the present invention the term “base material” is interchangeably use for “support”, “support material”, “carrier” or “carrier material”.

The base materials may consist of irregularly shaped or spherical particles, whose particle size is preferably between 2 and 1000 μm. Preference is given to particle sizes between 3 and 300 μm.

The base materials may, in particular, be in the form of non-porous, core-shell or preferably porous particles. The pore sizes is preferably between 2 and 300 nm. Preference is given to pore sizes between 5 and 200 nm.

The base materials may equally also be in the form of membranes, fibres, hollow fibres, coatings, filters, capillaries, surfaces, monoliths or monolithic mouldings. Preferably the base materials are beads or membranes, preferably porous beads or membranes. The base materials might also be made by additive manufacturing like 3D printing.

A membrane as base material can be distinguished from particle-based chromatography by the fact that the interaction between a solute, e.g. the target nucleic acids or contaminants, and the matrix does not take place in the dead-ended pores of a particle, but mainly in the throughpores of the membrane. Exemplary types of membranes are flat sheet systems, stacks of membranes, microporous polymer sheets with incorporated cellulose, polystyrene or silica-based membranes as well as radial flow cartridges, hollow fiber modules and hydrogel membranes. Preferred are hydrogel membranes. Such membranes comprise a membrane support and a hydrogel formed within the pores of said support. The membrane support provides mechanical strength to the hydrogel. The hydrogel determines the properties of the final product, like pore size and binding chemistry.

The membrane support can consist of any porous membrane like polymeric membranes, ceramic based membranes and woven or non-woven fibrous material. Suitable polymeric materials for membrane supports are preferably cellulose or cellulose derivatives as well as other preferably inert polymers like polyethylene, polypropylene, polybutylenterephthalate or polyvinylidene-difluoride.

Especially preferred are membranes made of an inert, flexible fiber web support comprising assembly within and around the fiber web support a porous polyacrylamide hydrogel with quaternary ammonium groups (strong anion exchange groups), like Natrix® Q Chromatography membrane, Merck KGaA, Germany.

Monoliths and monolithic mouldings are, three-dimensional bodies with throughpores, like interconnected channels, so that liquid can flow from one side of the monolith, through the monolith, to the other side of the monolith.

Since the mobile phase is flowing through these throughpores, molecules to be separated are transported by convection rather than by diffusion. Due to their structure monolithic sorbents show flow rate independent separation efficiency and dynamic capacity. The monolith is typically formed in situ from reactant solutions and can have any shape or confined geometry, typically with frit-free construction, which guarantees convenience of operation. Preferably, monolithic materials have a binary porous structure, mesopores and macropores. The micron-sized macropores are the throughpores and ensure fast dynamic transport and low backpressure in applications; mesopores contribute to sufficient surface area and thus high loading capacity.

Suitable organic polymers are polymethacrylates, polyacrylamides, polystyrenes, polyurethanes, etc., like Poly(methacrylic acid-ethylene dimethacrylate), Poly(glycidyl methacrylate-ethylene dimethacrylate) or Poly(acrylamide-vinylpyridine-N,N′-methylene bisacrylamide).

Inorganic monoliths can be made of silica or other inorganic oxides. Preferably they are made of silica. The monoliths can be made of organic, inorganic or organic/inorganic hybrid materials. Preferred are organic polymer based monoliths.

In one embodiment the base material is in the form of porous particles. The particles can be made of an inorganic or organic material or polymer.

In one embodiment the base material is an inorganic material, e.g. made of metal oxides like SiO₂, Al₂O₃, titanium dioxide, zirconium dioxide. It may also be made of other silica based materials like controlled pore glass.

The base material might also be an inorganic-organic hybrid material or any other combination of an organic and an inorganic material.

The base material might also be an organic material or polymer. In one embodiment, the base material is made from a natural polymer, preferably in the form of porous beads, e.g. polysaccharides based on agarose, cellulose, cellulose derivatives and polymers based on dextran. Natural polymer beads are e.g. of the kind known as Sepharose® or Sephadex®.

In one embodiment, the base material is made from a synthetic polymer, preferably in the form of porous beads or membranes, comprised of cross-linked synthetic polymers, such as styrene or styrene derivatives, divinylbenzene, acrylamides, acrylate esters, methacrylate esters, vinyl esters, vinyl amides etc. Polymers based on polystyrene, polyvinyl alcohol or copolymers of (meth)acrylate derivatives and comonomers with aliphatic hydroxyl groups are preferred. Polymers based on a certain type of structure like e.g. polystyrene or polyvinylether are polymers comprising said structure. They might also comprise other structures resulting e.g. from co-polymerisation of two different monomers.

In another preferred embodiment, the base material is a polyvinylether based material, especially a copolymer formed by copolymerisation of at least one compound from the group a) and b), whereas group a) is at least one alkyl vinyl ether of the formula (I)

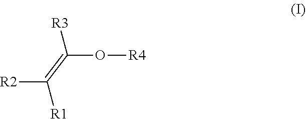

wherein,
R1, R2, R3 independently of one another represent H or C₁-C₆ alkyl, preferably H or —CH₃, and
R4 represents a substituent which carries at least one hydroxyl group and group b) is at least one crosslinking agent conforming to formula (II) and/or (III) and/or (IV)

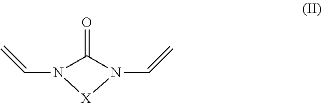

wherein,
X represents a divalent alkyl group having 2 to 5 C atoms, preferably 2 or 3 C atoms, in which one or more methylene groups which are not adjacent and are not located in the direct vicinity of N may be replaced by O, C═O, S, S═O, SO₂, NH, NOH or N and one or more H atoms of the methylene groups may be substituted, independently of one another, by hydroxyl, C₁-C₆ alkyl, halogen, NH₂, C₆-C₁₀ aryl, NH—(C₁-C₈)-alkyl, N—(C₁-C₅)-alkyl₂, C₁-C₆-alkoxy or C₁-C₆-alkyl-OH;

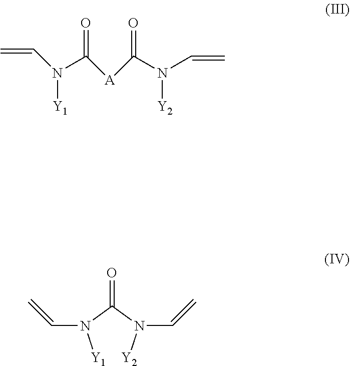

wherein
Y₁ and Y₂ in formula (III) and (IV) represent, independently of one another, C₁-C₁₀ alkyl or cycloalkyl, where one or more non-adjacent methylene groups or methylene groups which are not located in the direct vicinity of N may be replaced by O, C═O, S, S—O, SO₂, NH, NOH or N and one or more H of the methylene groups may be substituted, independently of one another, by hydroxyl, C₁-C₆ alkyl, halogen, NH₂, C₆-C₁₀-aryl, NH(C₁-C₅) alkyl, N(C₁-C₈) alkyl₂, C₁-C₆-alkoxy or C₁-C₆-alkyl-OH, or C₆-C₁₈ aryl, where one or more H in the aryl system may be substituted, independently of one another, by hydroxyl groups, C₁-C₆-alkyl, halogen, NH₂, NH(C₁-C₈) alkyl, N(C₁-C₅)alkyl₂, C₁-C₆-alkoxy or C₁-C₆-alkyl-OH, and
A is a divalent alkyl group having 2 to 5 C atoms, preferably 2 or 3 C atoms, in which one or more non-adjacent methylene groups or methylene groups which are not located in the direct vicinity of N may be replaced by O, C═O, S, S═O, SO₂, NH, NOH or N and one or more H of the methylene groups may be substituted, independently of one another, by hydroxyl groups, C₁-C₆ alkyl, halogen, NH₂, C₆-C₁₀-aryl, NH(C₁-C₅) alkyl, N(C₁-C₈)alkyl₂, C₁-C₆ alkoxy or C₁-C₆ alkyl-OH.

R4 in formula (I) is typically an alkyl, a cycloalkyl or an aryl which carries at least one hydroxyl group.

In a very preferred embodiment the matrix is formed by copolymerisation of an alkyl vinyl ether employed selected from the group of 1,4-butanediol monovinyl ether, 1,5-pentanediol monovinyl ether, diethylene glycol monovinyl ether or cyclo-hexanedimethanol monovinyl ether and divinylethyleneurea (1,3-divinylimidazolin-2-one) as crosslinking agent.

An example of a suitable commercially available vinylether based base material is Eshmuno®, Merck KGaA, Germany.

Further examples of suitable commercially available base materials are Macro-Prep CM Resin, Fractogel®, 2 and Sepharose®.

Boron clusters are attached to the base material as defined above. The boron cluster can be non-covalently or covalently bonded to the base material, preferably the boron cluster is covalently bonded to the base material.

In one embodiment the at least one boron cluster is bonded directly to the base material. The covalent attachment can for example be performed by directly bonding the boron cluster to suitable residues on the base material like OH, NH₂, carboxyl, phenol, anhydride, aldehyde, epoxide or thiol etc.

In a preferred embodiment the at least one boron cluster is bonded to the base material via a linker. According to the invention, the linker is a chemical group that connects the boron cluster and the base material. The chemical group can contain chemical moieties of the base material and/or the boron cluster, e.g. amine, hydroxy, epoxy or carboxy. Any linker suitable to connect the base material and the boron cluster can be used.

In one embodiment the linker is a structure of formula (V),

-X—Z—Y— (V)
wherein,
X and Y represent a first and a second reactive or activatable group, preferably, X and Y is independently selected from a moiety consisting of hydroxy, amino, thiol, carboxy, oxiranyl, formyl, halo, isocyanato and chloro sulphonyl; and
Z is selected from the groups consisting of
(a) C₁-C₁₅ alkyl,
(b) aryl,
(c) C₁-C₁₀ alkyl-aryl,
(d) C₁-C₁₀ alkyl-aryl-C₁-C₆ alkyl, wherein one or more of the carbon atoms of the alkyl may be replaced by an oxygen, sulfur or nitrogen, and wherein aryl includes but is not limited to phenyl, naphthyl, pyridyl or thienyl, and wherein one or more of the carbon atoms might be substituted with OH or C₁-C₆ alkyl, and
(e) a peptide of 2 to 10 amino acids, said amino acids included, but not limited to the L- and D-forms of the amino acids including glycine, alanine, valine, leucine, isoleucine, serine, threonine, aspartic acid, asparagine, glutamic acid, glutamine, lysine, hydroxy-lysine, histidine, arginine, phenylalanine, tyrosine, tryptophan, cysteine, methionine, ornithine, beta-alanine, homoserine, homotyrosine, homophenylalanine and citrulline.

Other linkers may include, but are not limited to p-benzoquinone, bis-(diazobenzidine), 3,6-bis-(mercurimethyl) dioxane, bisoxiranes, cyanuric chloride, dicyclohexylcarbodiimide, dinitrophenylsulphone, dimethyladipimidate, dimethylsuberimidate, divinylsulphone, N,N′-ethylene-bis-(iodoacetamide), glutaraldehyde, hexamethylene bis(maleimide), hexamethylene diisocyanate, N,N′-1,3-phenylene-bis-(maleimide), phenol-2,4-disulphonyl chloride, tetra-azotised o-dianisidine, toluene diisocyanate, Woodward's K reagent, water soluble carbodiimides, 6-aminohexanoic acid, hexamethylene-diamine, 1,7-diamino-4-aza-heptane (3,3′-diamino-dipropylamine), and aminoacids or peptides.

In one embodiment the structure of the linker is,

-Xₘ-(CH-A)ₙ-X-(CHA-CHB)-
wherein,
X represents O, NR′ or S, wherein R′ represents H or C₁-C₄ alkyl
A represents H or C₁-C₄ alkyl
B represents H or OH
n represents an integer of from 0 to 12
m represents the integer 1, or 0 in the case n represents the integer 0.

In one embodiment the structure of the linker is,

-Dₓ-(CHA-CHB)_{y}-X-Zₘ-(CHA-CH2)ₙ-O—
wherein,
X represents O or NR′, or X represents S in case Z represents CH2 or m represents the integer 0,
D represents O, NR′ or S,
Z represents CH2 or C═O,
A, B, R′ independently of one another represent H or C₁-C₄ alkyl,
n represents an integer of from 1 to 12
m represents the integer 0 or 1
x represents the integer 1 or x represents the integer 0 in the case y represents the integer 0
y represents an integer of from 0 to 4.

In one embodiment the structure of the linker is,

-Dₓ-(CHA-CHB)_{y}-[Oₖ-(CHA-CHB)]_{z}-X-Zₘ-(CHY—CH2)ₙ-O—
wherein,
X represents O or NR′ or X represents S in case Z represents CH2 or m represents the integer 0,
D represents O, NR′ or S,
Z represents CH₂ or C═O,
Y represents H, C₁-C₄ alkyl, —(CH₂-CH₂)ₚC(O) OH,
A, B, R′ independently of one another represent H or C₁-C₄ alkyl,
n represents an integer of from 1 to 12,
m, k, p independently of one another represent the integer 0 or 1,
x represents the integer 1, or x represents the integer 0 in the case y represents the integer 0,
y represents an integer of from 0 to 4 and
z represents an integer of from 1 to 4.

In one embodiment the structure of the linker is as structure selected from the group consisting of —CH(OH)CH₂—NH—, —O—(CH₂)₂—(C=O)—NH—, —O—(CH₂)₃-(C=O)—NH—(CH₂)₂-O—(CH₂)₂—O—, —O—(CH₂)₂—(C═O)NH—(CH₂)₄—O—, —O—(CH₂)₂—(C═O)NH—(CH₂)₂-O—(CH₂)₂—O— and —O—(CH₂)₂—O—(CH₂)₂—NH—(C=O)—(CH₂)₂—O—.

The stationary phase according to the present invention comprises a base material and at least one boron cluster as defined above. The stationary phase can comprise further functional groups beside the at least one boron cluster. This might for example be ionic, hydrophilic or hydrophobic groups. By generating a stationary phase with two different functionalities, one obtains mixed mode materials with separation properties resulting from both or several types of functionalities, whereby in case of the present invention at least one functional group is a boron cluster.

The stationary phases according to the invention can also be described as base materials provided with separation effectors whereby at least one of the separation effectors is a boron cluster as defined above.

In a preferred embodiment, the stationary phase comprises a base material which is formed by copolymerisation of an alkyl vinyl ether employed selected from the group of 1,4-butanediol monovinyl ether, 1,5-pentanediol monovinyl ether, diethylene glycol monovinyl ether or cyclo-hexanedimethanol monovinyl ether and divinylethyleneurea (1,3-divinylimidazolin-2-one) as crosslinking agent. More preferably, the base material is a suitable commercially available vinylether based base material, e.g. Eshmuno®, Merck KGaA, Germany.

In a preferred embodiment, the stationary phase comprises a boron cluster unit which is a closo-B₁₂X₁₁, a closo-B₁₀X₉, a nido-C₂B₉X₁₁ or a 3,3′-Co (1,2-C₂B₉X₁₀) (1′,2′-C₂B₉X₁₁) with X being a suitable substituent, e.g. hydrogen as in a closo-B₁₂H₁₁, a closo-B₁₀H₉, a nido-C₂B₉H₁₁ or a 3,3′-Co (1,2-C₂B₉H₁₀) (1′,2′-C₂B₉H₁₁, more preferably closo-B₁₂X₁₁, for example a closo-B₁₂H₁₁.

According to the invention, the term “group density” of the stationary phase refers to the density of the functional groups for binding of the boron cluster on the base material, e.g. COO⁻ group density of the base material starting material in μeq/g. According to the invention, the term “ligand density” of the stationary phase refers to the density of the boron cluster on the base material in μeq/g.

In a preferred embodiment, the stationary phase has a group density of from 300 to 2400 microequivalents per gram (μeq/g), preferably 700 to 1500 μeq/g, more preferably 1000 μeq/g.

In a preferred embodiment, the stationary phase has a ligand density of from 140 to 1500 microequivalents per gram (μeq/g), preferably 500 to 1000 μeq/g, more preferably 500 to 700 μeq/g, most preferably 621 μeq/g.

Increasing the group density or ligand density increased the amount of bound and eluted target molecule.

In a preferred embodiment, the stationary phase comprises irregularly shaped or spherical particles, whose particle size is between 2 and 1000 μm. Preferably, the particle size is between 3 and 600 μm, 3 and 300 μm, 20 to 150 μm, more preferably between 20 and 100 μm. The particles may be in the form of non-porous, core-shell or preferably porous particles. The pore sizes of the particles is preferably between 2 and 300 nm. Preferably the pore size is between 5 and 200 nm, more preferably between 40 and 110 nm.

In one embodiment the stationary phase is a combination of the preferred embodiments of the stationary phase as mentioned above can be combined.

The stationary phases according to the present invention can be made by different processes. The at least one boron cluster is either bonded directly to the base material or via a linker as described above. The covalent attachment can for example be performed by directly bonding via suitable residues on the base material. Suitable residues are known to the skilled person in the art, for example OH, NH₂, carboxyl, phenol, anhydride, aldehyde, epoxide or thiol. Exemplified syntheses are described in the literature, e.g. Graalfs, H.; Graft Copolymer for Cation-exchange Chromatography; WO2008145270.

It is also possible to generate the stationary phase according to the present invention by polymerizing monomers comprising a boron cluster and a polymerizable moiety. Examples of stationary phases generated by polymerization of suitable monomers are polystyrene, polymethacrylamide or polyacrylamide based stationary phases generated by polymerizing suitable styrene or acryloyl monomers.

In the case of “grafting to”, polymer chains must firstly be formed from the monomers and bound to the surface of the base material in a second step. In the case of “grafting from”, a polymerisation reaction is initiated on the surface of the base material, and the graft polymer is built up directly from individual monomers.

Preference is given to the “grafting from” method and particular preference is given to variants in which only a few by-products, such as a non-covalently bonded polymer, which have to be separated off are formed. Processes with controlled free-radical polymerisation, such as, for example, the method of atom-transfer free-radical polymerisation (ATRP), are suitable. Here, an initiator group is covalently bonded to the surface of the base material in the desired density in a first step. An initiator group is, for example, a halide bonded via an ester function, as in a 2-bromo-2-methylpropionic acid ester. The graft polymerisation is carried out in a second step in the presence of copper (I) salts.

In one embodiment the stationary phase is generated by grafting the boron cluster onto the base material. In a further embodiment the boron cluster are comprised in polymer chains, also called tentacles, grafted onto the base material.

Depending on the process conditions, the grafting with monomers leads to the coupling of one monomer or multiple monomers in a polymerization reaction forming a polymer chain. A polymer chain contains multiple monomers and can contain multiple boron clusters. In the interest of simplification, the resulting products of such grafting processes are displayed as a product obtained by the coupling of only one monomer throughout the experimental part, e.g. examples in sections 1.7, 1.8 and 1.10. It is emphasized that those products cover stationary phases resulting from the coupling of one monomer and multiple monomers, whereas the coupling of multiple monomers leads to polymer chains that can contain more than one boron cluster.

By way of example, example 1.8.3 is depicted with the following formula (VI)

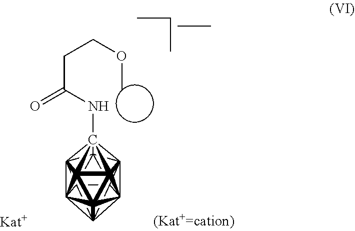

The Eshmuno®_{carboxy} starting material is synthesized via a grafting reaction including Eshmuno®_{hydroxy} and acrylic acid, as illustrated in section 1.8.1. The grafting reaction with the monomer acrylic acid can lead to the coupling of one acrylic acid or multiple acrylic acids in a polymerization reaction forming a polymer chain. A polymer chain contains two or more monomers with respective carboxy groups. Each of the carboxy groups can be coupled with an amine derivative of adamantane or closo-boron cluster under the protocol illustrated in sections 1.8.2 to 1.8.13. Hence, the polymer chain can contain multiple boron clusters. The following formula (VII) illustrates possible chemical structures of example 1.8.3.

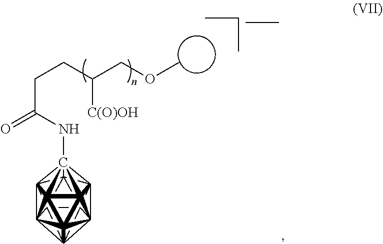

whereas n represents an integer of 0, 1, 2 or more. Preferably, n represents an integer of from 0 to 10. Formula (VII) is illustrated with one boron cluster but it is emphasized that each of the carboxy groups can be coupled with a boron cluster. According to the present invention, the illustration of example 1.8.3 with formula (VI) covers all the chemical structure as mentioned above. This applies equally to all examples in sections 1.8 and 1.10.

By way of example, example 1.7.3 is depicted with the following formula (VIII)

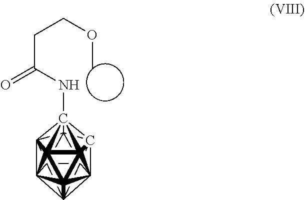

Formula (VIII) is synthesized via a grafting reaction including Eshmuno®_{hydroxy} and a monomer which is an acrylic acid or allylic derivative of the boron cluster (boron-containing monomer), as illustrated in section 1.7.1. The grafting reaction can lead to the coupling of one boron-containing monomer or multiple boron-containing monomers in a polymerization reaction forming a polymer chain. A polymer chain contains two or more monomers with respective boron cluster. Hence, the polymer chain contains multiple boron clusters. The following formula (IX) illustrates possible chemical structures of example 1.7.3.

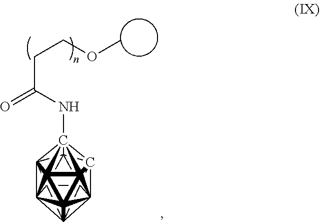

whereas n represents an integer of 1, 2 or more. Preferably, n represents an integer of from 1 to 10, more preferably 1 to 5. According to the present invention, the illustration of example 1.7.3 with formula (VIII) covers all the chemical structure as mentioned above. This applies equally to all examples in section 1.7.

A very preferred one-step grafting from polymerisation reaction suitable for the production of the stationary phase of the present invention can be initiated by cerium (IV) on a hydroxyl-containing support, without the support having to be activated.

This cerium (IV) initiated grafting is preferably carried out in accordance with EP 0337144 or U.S. Pat. No. 5,453,186. The chain produced is linked to the base material via a monomer unit. To this end, the base material according to the invention is suspended in a solution of monomers, preferably in an aqueous solution. The grafting-on of the polymeric material is effected in the course of a conventional redox polymerisation with exclusion of oxygen. The polymerisation catalyst employed is cerium (IV) ions, since this catalyst forms free-radical sites on the surface of the base material, from which the graft polymerisation of the monomers is started. This reaction is normally carried out in dilute mineral acids. In order to carry out this graft polymerisation, the acid is usually employed in an aqueous solution with a concentration in the range from 1 to 0.00001 mol/l, preferably from 0.1 to 0.001.

Very particular preference is given to the use of dilute nitric acid, which is employed with a concentration in the range from 0.1 to 0.001 mol/l.

For the preparation of the separating materials according to the invention, the monomers are normally added in excess to the base material. Typically, 0.05 to 100 mol of total monomer are employed per litre of sedimented polymer material, preferably 0.05-25 mol/l are employed.

The polymerisation is terminated by termination reactions involving the cerium salts. For this reason, the (average) chain length can be influenced by the concentration ratios of the base material, the initiator and the monomers. Furthermore, uniform monomers or also mixtures of different monomers can be employed; in the latter case, grafted copolymers are formed.

The monomers to be favourably used for the preparation of the separation materials according to the present invention are those according to formula (X)

CR*R**=CR¹-(CR²)_{z}—Y—B (X)
wherein
R*, R** and R¹ independently of one another represent H or CH₃,
R² is selected from a group consisting of H, alkyl, phenyl, cycloalkyl or alkyl-cycloalkyl, phenylalkyl or alkylphenyl group having up to 10 C-atoms in the alkyl group, it being possible for these groups to be monosubstituted or polysubstituted, preferably by halogen, alkoxy, cyano, amino, mono- or dialkylamino, trialkylammonium, carboxyl, sulfonyl, acetoxy or acetamino moieties,
a cyclic or bicyclic substituent having 5 to 10 C-atoms, wherein one or more CH or CH₂ groups can be replaced, by N or NH, N or NH and S, or N or NH and O,
or a sulfone sulfide of the structure —(CH₂)ₙ—SO₂—(CH₂)ₙ—S(CH₂)ₙOH, wherein n represents an integer of from 2 to 6
Y represents-C(═O)—X, —OC(═O)(CHR³)—, —(CH₂)ₘNH—, —(CH₂)ₘ—, wherein
   X is —NR⁴-, -NR⁴R⁵-, SR⁴, —S— or —O—, wherein
      R⁴ and R⁵ independently of one another are selected from a group consisting of alkyl, phenyl, cycloalkyl or alkyl-cycloalkyl, phenylalkyl or alkylphenyl group having up to 10 C-atoms in the alkyl group, it being possible for these groups to be monosubstituted or polysubstituted, preferably by halogen, alkoxy, cyano, amino, mono- or dialkylamino, trialkylammonium, carboxyl, sulfonyl, acetoxy or acetamino moieties, a cyclic or bicyclic substituent having 5 to 10 C-atoms, wherein one or more CH or CH₂ groups can be replaced, by N or NH, N or NH and S, or N or NH and O, or a sulfone sulfide of the structure —(CH₂)ₙ—SO₂—(CH₂)ₙ—S(CH₂)ₙOH and one of the substituent R² and R³ may also be H, where R² and R³ are co-ordinated with one another so that either both substituents are acidic or basic or one or both of the substituents are neutral, wherein
      n represents an integer of from 2 to 6
   R³ is selected from H or an alkyl group having up to 5 C-atoms,
   m represents an integer of from 1 to 12,
z represents an integer of from 0 to 6, and
B represents a boron cluster according to the present invention.

Preferably, the monomers used for the preparation of the separation materials according to the present invention are those according to formula (XI)

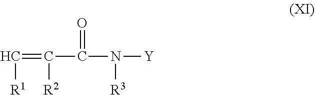

wherein
R¹, R² and R³ are independently selected from H or CH₃, preferably H, Y represents a boron cluster according to the present invention.

The separation materials according to the present invention preferably only contain tentacle-like linear polymer structures grafted onto the base material that are built from monomers according to formula (X). Preferably, they contain linear polymers that are only build by one type of monomer according to formula (X).

But it is also possible that the linear polymers are built by co-polymerization of two or more different monomers according to formula (X). It is also possible that the linear polymers are built by co-polymerization of one or more different monomers according to formula (X) and one or more other polymerizable monomers like other acrylamides, methacrylates, acrylates, methacrylates etc. which are functionalized e.g. with ionic, hydrophilic or hydrophobic groups.

A boron cluster as described above, the stationary phases comprising the boron cluster and the separation device comprising the stationary phase according to the invention is preferably used for the selective, partially selective or non-selective binding, adsorption or purification of one or more target molecules with the aim of separation out of a sample liquid, or for the selective, partially selective or non-selective binding or adsorption of one or more secondary components with the aim of separation of the secondary component out of a matrix, the isolation, enrichment and/or depletion of biopolymers from natural sources, the isolation, enrichment and/or depletion of biopolymers from recombinant sources, the isolation, enrichment and/or depletion of proteins and peptides, the isolation, enrichment and/or depletion of monoclonal and polyclonal antibodies, the isolation, enrichment and/or depletion of viruses, the isolation, enrichment and/or depletion of host cell proteins, the isolation, enrichment and/or depletion of ADCs, the isolation, enrichment and/or depletion of alkaloids, lipids like diglycerides or triglycerides, carbohydrates, nucleic acids or other biomolecules. Preferably the target molecule is a protein. Preferably the separation and/or purification of a target molecule is a solid phase extraction or chromatographic separation, more preferably a chromatographic separation.

In one embodiment a boron cluster, the stationary phase according to the invention or the separation device according to the invention is used for separation and/or purification of a target molecule, preferably a protein.

In one embodiment the separation and/or purification of a target molecule is a solid phase extraction or chromatographic separation, preferably a chromatographic separation.

The target molecules are separated from at least one or more other substances from a sample, whereby the sample which comprises the target molecule is a liquid or is dissolved in a liquid, which is brought into contact with the stationary phase according to the invention. Contact times are usually in the range from 30 seconds to 24 hours. It is advantageous to work in accordance with the principles of liquid chromatography by passing the liquid through a chromatography column which contains the stationary phase according to the invention. The liquid can run through the column merely through its gravitational force or it can be pumped through by means of a pump. An alternative method is batch chromatography, in which the stationary phase is mixed with the liquid by stirring or shaking for as long as the target molecules need to be able to bind to the stationary phase. It is likewise possible to work in accordance with the principles of the chromatographic fluidized bed by introducing the liquid to be separated into, for example, a suspension comprising the stationary phase, where the separating material is selected so that it is suitable for the desired separation owing to its high density and/or a magnetic core.

If the chromatographic process is run in the bind and elute mode, the target molecule binds to the stationary phase according to the invention. The stationary phase can optionally, subsequently be washed with a wash buffer, which preferably has the same ion strength and the same pH as the liquid in which the target molecule is brought into contact with the stationary phase. The wash buffer removes all substances which do not bind to the stationary phase. Further washing steps with other suitable buffers may follow without desorbing the target molecule. The desorption of the bound target molecule is typically carried out by changing the ion strength in the eluent and/or by changing the pH in the eluent and/or by changing the solvents. The target molecule can thus be obtained in a purified and concentrated form in the eluent. The target molecule usually has a purity of 70 percent to 99 percent, preferably 85 percent to 99 percent, particularly preferably 90 percent-99 percent, after desorption from the stationary phase.

However, if the chromatographic process is run in the flow-through mode, the target molecule remains in the liquid phase, but other accompanying substances bind to the stationary phase. The target molecule is then obtained directly by collecting the column eluate in through-flow.

The stationary phases according to the present invention can be used for plenty of different applications. They can for example be used for hydrophilic and hydrophobic separations as well as for aqueous and non-aqueous separations.

Without further elaboration, it is believed that one skilled in the art can, using the preceding description, utilise the present invention to its fullest extent. The preferred specific embodiments and examples are, therefore, to be construed as merely illustrative, and not limiting to the remainder of the disclosure in any way whatsoever.

The present invention if further directed to a process for the separation of a target molecule from at least one other compound whereby a stationary phase according to the present invention as described above, preferably present in a separation device, is contacted with a liquid comprising the target molecule and the at least one other compound and whereby the target molecule shows a binding to the stationary phase that is different from the binding of the other compound, e.g. it interacts stronger or weaker with the stationary phase than the other compounds. The process is preferably used in a process for solid phase extraction or chromatographic separation.

In another embodiment the process is a process for chromatographic separation of a target molecule from at least one other compound whereby a stationary phase according to the present invention as described above is present in a chromatography column and the liquid comprising the target molecule and the at least one other compound is run through the column whereby the target molecule and the other compounds present in the liquid are eluted from the column depending on their interaction with the stationary phase.

In another embodiment the process is performed by loading the stationary phase according to the present invention with an aqueous loading buffer of a certain pH and a certain ionic strength and eluting the target molecule with an aqueous elution buffer of the same pH but another ionic strength, typically a higher ionic strength.

In another embodiment the process is performed by loading the stationary phase according to the present invention with an aqueous loading buffer of a certain pH and a certain ionic strength and eluting the target molecule with an aqueous buffer of another pH and/or another ionic strength, optionally with a further additive.

In another embodiment the process is performed by loading the stationary phase according to the present invention with an aqueous loading buffer, having a pH value in the range of 2 to 11, preferably 5 to 9, more preferably 4.5 to 7.5, most preferably having a pH value of 6, and a conductivity in the range of 1 to 150 mS/cm, preferably in the range of 2 to 100 mS/cm and eluting the target molecule with an aqueous buffer of another pH and/or another ionic strength, optionally with a further additive.

In another embodiment the process is performed by loading the stationary phase according to the present invention with an aqueous loading buffer, having a pH value of 6, and eluting the target molecule with an aqueous buffer, having a pH value of 10, with imidazole, preferably 3M solution of imidazole as further additive.

In another embodiment the process is performed by loading the stationary phase according to the present invention with an aqueous loading buffer of a certain pH and a certain ionic strength and eluting the target molecule with an organic medium. An organic medium is an organic liquid that does not comprise more than 10% of water, e.g. methanol, ethanol, acetonitrile, THF, heptane, toluene etc. or mixtures thereof.

In another embodiment the process is performed by loading the stationary phase according to the present invention with an organic loading medium and eluting the target molecule with the same medium or a more polar medium, e.g. using a gradient elution. An organic loading medium for this embodiment is a liquid comprising water and an organic solvent with a maximum of 20% of the organic solvent. Various organic solvents can be selected, e.g. methanol, ethanol, acetonitrile, THF, heptane, toluene etc. or mixtures thereof.

The present invention is further directed to the chromatographic separation of two proteins having a different pI (isoelectric point) and/or a different content of carboxylic acids by boron cluster, preferably using a stationary phase according to the present invention.

The present invention is further directed to a separation device comprising the stationary phases according to the present invention. The device can for example be used for solid phase extraction or for chromatographic applications. In any case it comprises a means for holding the stationary phase. The stationary phase might be surrounded by the device or attached to it.

In one embodiment, the device comprises a housing with an inlet and an outlet. In another embodiment it is a flat plate or a pin with the stationary phase attached to one side of it. In another embodiment it is a filter comprising the stationary phase. In a preferred embodiment the device is a chromatography column comprising the above described stationary phases according to the present invention. Chromatography columns are known to a person skilled in the art. They typically comprise cylindrical tubes or cartridges filled with the stationary phase as well as filters and/or means for fixing the stationary phase in the tube or cartridge and optionally connections for solvent delivery to and from the tube or cartridge. The size of the chromatography column varies depending on the application, e.g. analytical or preparative. In one embodiment the column or generally the separation device is a single use device.

### EXAMPLES

### 1. Synthesis of Chromatography Material

### 1.1 Synthesis of Functionalized boron Clusters of the General Formula: boron-cluster-O-CH₂CH₂-Y-CH₂CH₂—NH₂ (Y=O, or No Atom), General Protocol for the Synthesis of the Respective tetrabutylammonium salt

The conversion of dioxane and tetrahydrofuran derivatives of closo-dodecaborate and closo-decaborate anions with ammonia were carried out similar to the reaction of [nBu₄N] [1-O(C₂H₄)₂O-closo-B₁₂H₁₁] with ammonia as described in the literature (Sivaev, I. B.; Semioshkin, A. A.; Brellochs, B.; Sjöberg, S.; Bregadze, V. I. Synthesis of oxonium derivatives of the dodecahydro-closo-dodecaborate anion [B₁₂H₁₂]²⁻. Tetramethylene oxonium derivative of [B₁₂H₁₂]²⁻ as a convenient precursor for the synthesis of functional compounds for boron neutron capture therapy. Polyhedron 2000, 19, 627-632; Ishii, S.; Nakamura, H. Synthesis and biological evaluation of closo-dodecaborate ibuprofen conjugate (DIC) as a new boron agent for neutron capture therapy. J. Organomet. Chem. 2018, 865, 178-182; Kubasov, A. S.; Matveev, E. Y.; Retivov, V. M.; Akimov, S. S.; Razgonyaeva, G. A.; Polyakova, I. N.; Votinova, N. A.; Zhizhin, K. Y.; Kuznetsov, N. T. Nickel (II) complexes with nitrogen-containing derivatives of the closo-decaborate anion. Russ. Chem. Bull. 2014, 63, 187-193.).

Here, the respective dioxane and tetrahydrofuran derivatives of closo-dodecaborate and closo-decaborate anions were suspended in ethanol and mixed with acetonitrile until the boron cluster derivatives were completely dissolved. Then, this solution was mixed with an aqueous solution of ammonia (25% solution) and stirred for 12 hours at 110° C. The solvent was removed in vacuum, the residue was dissolved in methanol and mixed with a tetrabutylammonium hydroxide solution (1 M in methanol). The solvent was removed under reduced pressure and the residue was taken up into dichloromethane. The organic phase was washed with water and dried over magnesium sulfate. The solvent was removed under reduced pressure and the solid obtained was dried in a fine vacuum.

Metathesis to cesium salt:

The tetrabutylammonium salt was dissolved in dichloromethane and mixed with a solution of cesium fluoride in methanol. The precipitant was filtered off, washed with dichloromethane and methanol, and dried in a fine vacuum.

### 1.1.1 Syntheses of Kat₂[1-H₂N(CH₂)₂O(CH₂)₂O-closo-B₁₂H₁₁] (Kat=[nBu₄N], Cs)

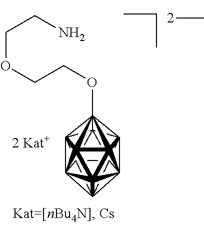

Quantities of Chemicals used for these Syntheses:
Kat=[nBu₄N]:
16.2 g (34.4 mmol) [nBu₄N] [1-O(CH₄)₂O-closo-B₁₂H₁₁];
500 mL of ethanol;
200 ml of a 25% aqueous solution of ammonia;
50 ml of acetonitrile;
100 mL of methanol;
41.2 mL of a tetrabutylammonium hydroxide solution (1 M in methanol);
100 mL of dichloromethane.
Yield of [nBu₄N]₂ [1-H₂N(CH₂)₂O(CH₂)₂O-closo-B₁₂H₁₁]: 24.7 g (33.9 mmol, 99%),
white solid.

Metathesis to Cs₂ [1-H₂N(CH₂)₂O(CH₂)₂O-closo-B₁₂H₁₁]:
24.7 g (33.9 mmol) of [nBu₄N]₂[1-H₂N(CH₂)₂O(CH₂)₂O-closo-B₁₂H₁₁] dissolved in 100 mL of dichloromethane;
11.4 g (74.5 mmol) of CsF in 150 mL of methanol.
Washing with 3×50 mL dichloromethane and 3×50 mL methanol.
Yield: 16.1 g (31.6 mmol, 93%), white solid.

### Characterization:

NMR data of the [1-H₂N(CH₂)₂O(CH₂)₂O-closo-B₁₂H₁₁]²⁻ anion:

¹H NMR (500.1 MHZ, CD₃CN): δ=3.74-3.66 (m, 6H, 3CH₂), 3.05 (t, 2H, CH₂, ³J_{HH}=5.14 Hz), 1.82-0.69 (m, 11H, BH) ppm, signal of NH was not observed.

¹H {¹¹B} NMR (500.1 MHZ, CD₃CN): δ=3.74-3.66 (m, 6H, 3CH₂), 3.05 (t, 2H, CH₂, ³J_{HH}=5.14 Hz), 1.14 (s, 5H), 0.81 (s, 5H), 0.55 (s, 1H) ppm, signal of NH was not observed.

¹¹B NMR (160.4 MHZ, CD₃CN): δ=6.4 (s, 1B), −16.7 (d, 5B, ¹J_{BH}=124 Hz), −18.1 (d, 5B, ¹J_{BH}=127 Hz), −22.8 (d, 1B, ¹J_{BH}=124 Hz) ppm.

¹¹B{¹H} NMR (160.4 MHZ, CD₃CN): δ=6.4 (s, 1B, B1), −16.7 (s, 5B), −18.1 (s, 5B), −22.8 (s, 1B) ppm.

¹³C{¹H} NMR (125.7 MHz, CD₃CN): δ=71.0 (s, 1C), 68.8 (s, 1C), 67.8 (s, 1C), 67.5 (s, 1C) ppm.

IR (ATR): [symbol] =2959 (m, v(C—H)), 2872 (m, v(C—H)), 2458 (s, v(B—H)) cm⁻¹.

### Elemental Analysis:

Calculated for [nBu₄N]₂[1-H₂N(CH₂)₂O(CH₂)₂O-closo-B₁₂H₁₁]: C, 59.24; H, 12.84; N, 5.76%. found for C₃₆H₉₃B₁₂N₃O₂: C, 58.37; H, 12.73; N, 5.34%.

### 1.1.2 Synthesis of Kat₂[1-H₂N(CH₂)₄O-closo-B₁₂H₁₁] (Kat=[nBu₄N], Cs)

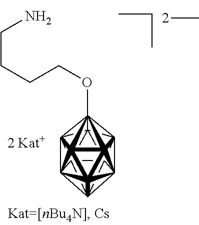

Quantities of Chemicals used for these Syntheses:

Kat=[nBu₄N]:
650 mg (1.42 mmol) [nBu₄N][1-(CH₂)₄O-closo-B₁₂H₁₁];
15 mL ethanol,
5 mL of an 25% aqueous solution of ammonia;
5 mL of acetonitrile;
10 mL of methanol,
3.20 mL of a tetrabutylammonium hydroxide solution (1 M in methanol);
25 mL of dichloromethane.
Yield of the [nBu₄N]₂ [1-H₂N(CH₂)₄O-closo-B₁₂H₁₁]: 920 mg (1.29 mmol, 91%), white solid.
   Metatheses to Cs₂[1-H₂N(CH₂)₄O-closo-B₁₂H_{11]:}
743 mg (1.04 mmol) of [nBu₄N] 2 [1-H₂N(CH₂)₄O-closo-B₁₂H₁₁] dissolved in 10 mL of dichloromethane;
347 mg (2.28 mmol) of CsF in 15 mL of methanol.
Washing with 3×5 mL dichloromethane and 3×5 mL methanol.
Yield of the Cs₂[1-H₂N(CH₂)₄O-closo-B₁₂H₁₁]: 320 mg (0.81 mmol, 77%), white solid.

### Characterization:

NMR data of the [1-H₂N(CH₂)₄O-closo-B₁₂H₁₁]²⁻ anion:

¹H NMR (500.1 MHz, CD₃CN): δ=3.45 (t, 2H, CH₂, ³J_{HH}=6.5 Hz), 2.71 (t, 2H, CH₂, ³J_{HH}=6.4 Hz), 1.82-0.69 (m, 11H, BH), 1.58-1.53 (m, 4H, 2CH₂) ppm, signal of NH was not observed.

¹H {¹¹B} NMR (500.1 MHZ, CD₃CN): δ=3.45 (t, 2H, ³J_{HH}=6.5 Hz, H₄), 2.71 (t, 2H, ³J_{HH}=6.4 Hz, H₁), 1.58-1.53 (m, 4H, H₂-3), 1.14 (s, 5H, B2-6H), 0.81 (s, 5H, B7-11H), 0.55 (s, 1H, B12H) ppm, signal of NH was not observed.

¹¹B NMR (160.4 MHZ, CD₃CN): δ=6.5 (s, 1B), −16.7 (d, 5B, ¹J_{BH}=124 Hz), −18.3 (d, 5B, ¹J_{BH}=127 Hz), −22.8 (d, 1B, ¹J_{BH}=124 Hz) ppm.

¹¹B {1H} NMR (160.4 MHZ, CD₃CN): δ=6.5 (s, 1B), −16.7 (s, 5B), −18.3 (s, 5B), −22.8 (s, 1B) ppm.

¹³C{1H} NMR (125.7 MHZ, CD₃CN): δ=49.8 (s, 1C), 46.5 (s, 1C), 28.1 (s, 1C), 23.1 (s, 1C) ppm.

IR (ATR): [symbol] =2900 (m, n (C—H)), 2861 (m, n (C—H)), 2471 (s, n (B—H) cm⁻¹.

### Elemental Analysis:

Calculated for Cs₂ [1-H₂N(CH₂)₄O-closo-B₁₂H₁₁]: C, 9.71; H, 4.28; N, 2.83%.

Found for C₄H₂₁B₁₂Cs₂NO: C, 10.95; H, 4.85; N, 2.64%.

### 1.1.3 Synthesis of Kat₂[2-H₂N(CH₂)₂O(CH₂)₂O-closo-B₁₀H₉] (Kat=[nBu₄N], Cs)

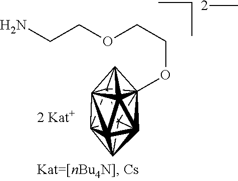

Quantities of Chemicals used for these Syntheses:

Kat=[nBu₄N]:
447 mg (0.99 mmol) [nBu₄N] [2-O(C₂H₄)₂O-closo-B₁₀H₉];
25 mL of ethanol;
10 mL of an 25% aqueous solution of ammonia;
15 mL of acetonitrile,
20 mL of methanol,
2.40 mL of a tetrabutylammonium hydroxide solution (1 M in methanol);
25 mL of dichloromethane.
Yield of [nBu₄N] 2 [2-H₂N(CH₂)₂O(CH₂)₂O-closo-B₁₀H₉]: 690 mg (0.97 mmol, 98%), white solid.

Metatheses to Cs₂ [2-H₂N(CH₂)₂O(CH₂)₂O-closo-B₁₀H₉]:
690 mg (0.97 mmol) of [nBUN] [2-H₂N(CH₂)₂O(CH₂)₂O-closo-B₁₀H₉] dissolved in 10 mL of dichloromethane;
230 mg (1.52 mmol) of CsF in 15 mL of methanol.
Washing with 3×5 mL dichloromethane and 3×5 mL methanol.
Yield of Cs₂ [2-H₂N(CH₂)₂O(CH₂)₂O-closo-B₁₀H₉]: 200 mg (0.41 mmol, 42%), white solid.

### Characterization:

NMR data of the [2-H₂N(CH₂)₂O(CH₂)₂O-closo-B₁₀H₉]²⁻ anion:

¹H NMR (500.1 MHz, CD₃CN): δ=3.66-3.59 (m, 6H, 3CH₂), 3.01 (t, 2H, CH₂, 3J_{HH}=5.30 Hz), 1.60-0.74 (m, 9H, BH) ppm, signal of NH was not observed.

¹H {¹¹B} NMR (500.1 MHz, CD₃CN): δ=3.66-3.59 (m, 6H, 3CH₂), 3.01 (t, 2H, CH₂ ³J_{HH}=5.30 Hz), 2.96 (s, 1H), 0.87 (s, 2H), 0.44 (s, 2H), 0.12 (s, 2H), −0.37 (s, 1H) ppm, signal of NH was not observed.

¹¹B NMR (160.4 MHZ, CD₃CN): δ=−1.8 (s, 1B), −3.2 (d, 1B, ¹J_{BH}=153 Hz), −5.7 (d, 1B, ¹J_{BH}=144 Hz), −23.8 (d, 4B, ¹J_{BH}=143 Hz), −29.6 (d, 2B, ¹J_{BH}=140 Hz,), −34.4 (d, 1B, ¹J_{BH}=136 Hz) ppm.

¹¹B {1H} NMR (160.4 MHZ, CD₃CN): δ=−1.8 (s, 1B), −3.2 (s, 1B), −5.7 (s, 1B), −23.8 (s, 4B), −29.6 (s, 2B), −34.4 (s, 1B) ppm.

¹³C{1H} NMR (125.7 MHZ, CD₃CN): δ=71.2 (s, 1C), 71.0 (s, 1C), 69.1 (s, 1C), 58.0 (s, 1C) ppm.

IR (ATR): [symbol] =2959 (m, n (C—H)), 2871 (m, n (C—H)), 2437 (s, n (B—H)) cm⁻¹.

### Elemental Analysis:

Calculated for Cs₂ [2-H₂N(CH₂)₂O(CH₂)₂O-closo-B₁₀H₉]: C, 9.86; H, 3.93; N, 2.88%.

Found for C₄H₁₉B₁₀Cs₂NO₂: C, 10.34; H, 4.02; N, 2.93%.

### 1.1.4 Syntheses of Kat₂[2-H₂N(CH₂)₄O-closo-B₁₀H₉] (Kat=[nBu₄N], Cs)

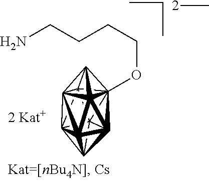

Quantities of Chemicals used for these Syntheses:

Kat=[nBu₄N]:
650 mg (1.51 mmol) [nBu₄N] [2-(CH₂)₄O-closo-B₁₀H₉];
15 mL of ethanol,
5 mL of an 25% aqueous solution of ammonia;
5 mL of acetonitrile;
10 mL of methanol,
3.30 mL of a tetrabutylammonium hydroxide solution (1 M in methanol);
25 mL dichloromethane.
Yield of [nBu₄N] 2 [1-H₂N(CH₂)₄O-closo-B₁₀H₉]: 966 mg (1.40 mmol, 93%), white solid.
   Metatheses to Cs₂ [1-H₂N(CH₂)₄O-closo-B₁₀H₉]:
1.00 g (1.45 mmol) of [nBu₄N] 2 [1-H₂N(CH₂)₄O-closo-B₁₀H₉] dissolved in 10 mL of dichloromethane;
484 mg (3.19 mmol) CsF in 15 mL of methanol.
Washing with 3×5 mL dichloromethane and 3×5 mL methanol.
Yield of Cs₂[1-H₂N(CH₂)₄O-closo-B₁₀H₉]: 360 mg (0.76 mmol, 53%), white solid.

### Characterization:

NMR data of the [1-H₂N(CH₂)₄O-closo-B₁₀H₉]²⁻ anion:

¹H NMR (500.1 MHZ, CD₃CN): δ=3.33 (t, CH₂, ³J_{HH}=5.0 Hz), 2.90 (t, CH₂, 3 J_{HH}=5.7 Hz), 1.60-0.74 (m, 9H, BH), 1.12-1.08 (m, 2CH₂,) ppm, signal of NH was not observed.

¹H {¹¹B} NMR (500.1 MHz, CD₃CN): δ=3.33 (t, CH₂, ³J_{HH}=5.0 Hz), 2.96 (s, 1H, BH), 2.90 (t, CH₂, 3J_{HH}=5.7 Hz), 1.12-1.08 (m, 2CH₂)), 0.87 (s, 2H, BH), 0.44 (s, 2H, BH), 0.12 (s, 2H, BH), −0.37 (s, 1H, BH) ppm, signal of NH was not observed.

¹¹B NMR (160.4 MHZ, CD₃CN): δ=−1.8 (s, 1B), −3.4 (d, 1B, ¹J_{BH}=153 Hz), −5.8 (d, 1B, ¹J_{BH}=144 Hz), −23.8 (d, 4B, ¹J_{BH}=143 Hz), −30.0 (d, 2B, ¹J_{BH}=140 Hz), −34.4 (d, 1B, ¹J_{BH}=136 Hz) ppm.

¹¹B{¹H} NMR (160.4 MHZ, CD₃CN): δ=−1.8 (s, 1B), −3.4 (s, 1B), −5.8 (s, 1B), −23.8 (s, 4B), −30.0 (s, 2B), −34.4 (s, 1B) ppm.

¹³C{¹H} NMR (125.7 MHZ, CD₃CN): δ=50.3 (s, 1C), 48.2 (s, 1C), 28.2 (s, 1C), 22.9 (s, 1C) ppm.

IR (ATR): [symbol] =2958 (m, n (C—H)), 2872 (m, n (C—H)), 2439 (s, n (B—H)) cm⁻¹.

### 1.1.5 Syntheses of [3,3′-Co (8-{NH₃CH₂CH₂OCH₂CH₂O}-1,2-C₂B₉H₁₀) (1′,2′-C₂B₉H₁₁)]

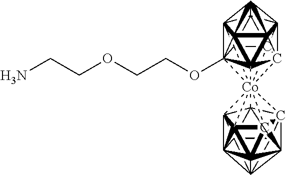

### Method A

The synthesis was performed in analogy to a literature procedure (Kvasničková, E.; Masák, J.; Čejka, J.; Maťátková, O.; Šícha, V. Preparation, characterization, and the selective antimicrobial activity of N-alkylammonium 8-diethyleneglycol cobalt bis-dicarbollide derivatives. J. Organomet. Chem. 2017, 827, 23-31.). [3,3′-Co (8-{O(CH₂CH₂)₂O}-1,2-C₂B₉H₁₀) (1′,2′-C₂B₉H₁₁)] (500 mg, 1.2 mmol) was dissolved in dry THF (20 mL). The orange solution was cooled to 0° C. and gaseous ammonia was bubbled through the solution. After 5 minutes, the ammonia flow was stopped and the reaction mixture was stirred for another 25 minutes. Afterwards, the solvent was evaporated and the residue was dissolved in diethyl ether and washed with aqueous HCl (3×20 mL, 20% v/v) and water (1×20 mL). The organic layer was separated and diethyl ether was removed under reduced pressure. Yield of [3,3′-Co (8-{NH₃CH₂CH₂OCH₂CH₂O}-1,2-C₂B9H₁₀) (1′,2′-C₂B9H₁₁)]: 480 mg (1.122 mmol, 92%) of an orange solid.

### Method B

[3,3′-Co (8-{O(CH₂CH₂)₂O}-1,2-C₂B9H₁₀) (1′,2′-C₂B9H₁₁)] (211 mg, 513.6 μmol) was dissolved in acetonitrile (11 mL) and aqueous ammonia (25%, 8 mL) was added. The orange solution was stirred 17 hours at 55° C. The solvent was evaporated under reduced pressure.

Yield of [3,3′-Co (8-{NH₃CH₂CH₂OCH₂CH₂O}-1,2-C₂B₉H₁₀) (1′,2′-C₂B₉H₁₁)]: 200 mg (467 μmol, 91%) of an orange solid.

### Characterization:

¹H {¹¹B} NMR (400.3 MHZ, CD₃CN): δ=6.61 (s, 3H, NH₃), 3.97 (s, 2H, C_{cluster}H), 3.88 (s, 2H, C_{cluster}H), 3.73 (m, 4H, CH₂), 3.60 (m, 2H, CH₂), 3.13 (t, 2H, CH), 3.08 (s, 1H, BH), 2.84 (s, 3H, BH), 2.61 (s, 3H, BH), 2.04 (s, 2H, BH), 1.80 (s, 2H, BH), 1.65 (s, 1H, BH), 1.56 (s, 2H, BH), 1.48 (s, 2H, BH), 1.40 (s, 1H, BH) ppm.

¹¹B{¹H} NMR (128.4 MHZ, CD₃CN): δ=24.38 (s, 1B), 6.71 (s, 1B), 0.14 (s, 1B), −2.81 (s, 1B), −5.45 (s, 2B), −6.97 (s, 4B), −9.71 (s, 2B), −17.56 (s, 2B), −20.49 (s, 2B), −22.83 (s, 1B), −29.14 (s, 1B) ppm.

¹¹B NMR (128.4 MHZ, CD₃CN): δ=24.34 (s, 1B), 6.60 (d, 1B, ¹J (11B, ¹H)=135 Hz), 0.33 (d, 1B, ¹J (11B, ¹H)=135 Hz), −2.65 (d, 1B, ¹J (11B, ¹H)=142 Hz), −5.46 (d, 2B, ¹J (11B, ¹H)=192 Hz), −6.76 (d, 4B, ¹J (11B, ¹H)=134 Hz), −9.35 (d, 2B, ¹J (11B, ¹H)=148 Hz), −17.29 (d, 2B, ¹J (¹¹B,¹H)=153 Hz), −20.23 (d, 2B, ¹J (¹¹B,¹H)=154 Hz), −22.28 (d, 1B, 178 Hz), −28.66 (d, 1B, ¹J (¹¹B,¹H)=181 Hz) ppm.

HRMS (ESI): found: 427.3475 (100), 426.3515 (96), 428.3439 (78), 425.3552 (61), 429.3403 (36), 424.3589 (35); calculated: 427.3476 (100), 426.3512 (99), 425.3548 (95), 428.3439 (79), 424.3585 (59), 429.3403 (47).

### Elemental Analysis:

Calculated for C₈H₃₂B₁₈CoNO₂: C: 22.46%, H: 7.34% N: 3.27%.

Found: C: 22.70%, H: 6.86%, N: 2.95%.

### 1.1.6 Syntheses of 10-{O(CH₂CH₂)₂O}-nido-7,8-C₂B₉H₁₂

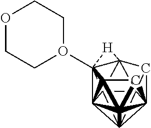

The synthesis was performed in analogy to a literature procedure (Řezáčová, P.; Pokorná, J.; Brynda, J.; Kožíšek, M.; Cígler, P.; Lepšík, M.; Fanfrlík, J.; Řezáč, J.; Grantz Šašková, K.; Sieglová, I.; Plešek, J.; Sícha, V.; Grüner, B.; Oberwinkler, H.; Sedláček, J.; Krausslich, H.-G.; Hobza, P.; Král, P.; Konvalinka, J. Design of HIV Protease Inhibitors Based on Inorganic Polyhedral Metallacarboranes. J. Med. Chem. 2009, 52, 7132-7141.).

[Et₃NH] [7,8-C₂B₉H₁₂] (1.27 g, 5.39 mmol) was suspended in toluene (9 mL) and concentrated sulfuric acid (2.2 mL) was added. The two-phase system was vigorously stirred for 10 minutes. The toluene layer was separated and the sulfuric acid phase was extracted with one more portion of toluene (4 mL). Dioxane (922.2 μL, 10.78 mmol) was added to the collected toluene phases. The clear solution was stirred for 22 hours at 80° C. Afterwards the solvent was evaporated under reduced pressure.

Yield of 10-{O(CH₂CH₂)₂O]-nido-7,8-C₂B⁹H₁₂: 890 mg (4.04 mmol, 75%) of a white solid.

### Characterization:

1H {¹¹B} NMR (400.3 MHZ, CD₂Cl₂): δ=4.51 (t, 4H, CH₂), 3.94 (t, 4H, CH₂), 2.39 (s, 2H, BH), 2.01 (s, 2H, C_{cluster}H), 1.68 (s, 1H, BH), 1.62 (s, 2H, BH), 1.36 (s, 2H, BH), 0.23 (s, 1H, m-BH) ppm.

¹¹B{¹H} NMR (128.4 MHZ, CD₂Cl²): δ=−9.45 (s, 1B), −12.52 (s, 2B), −17.06 (s, 2B), −22.06 (s, 3B), −39.74 (s, 1B) ppm.

¹¹B NMR (128.4 MHZ, CD₂Cl₂): δ=−9.34 (s, 1B), −12.46 (d, 2B, ¹J (¹¹B,¹H)=143 Hz), −16.96 (d, 2B, ¹J (¹¹B,¹H)=137 Hz), −21.89 (d, 3B, ¹J (¹¹B,¹H)=152 Hz), −39.57 (d, 1B, ¹J (¹¹B,¹H)=144 Hz) ppm.

### 1.1.7 Syntheses of 10-(NH₃CH₂CH₂OCH₂CH₂O)-nido-7,8-C₂B₉H₁₁

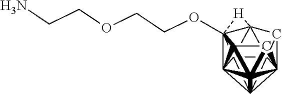

The synthesis was performed in analogy to a literature procedure (Bakardjiev, M.; Anwar, S. E.; Bavol, D.; Růžičková, Z.; Grüner, B. Focus on Chemistry of the 10-Dioxane-nido-7,8-dicarba-undecahydrido Undecaborate Zwitterion; Exceptionally Easy Abstraction of Hydrogen Bridge and Double-Action Pathways Observed in Ring Cleavage Reactions with OH⁻ as Nucleophile. Molecules 2020, 25, 814.). 10-{O(CH₂CH₂)₂O]-nido-7,8-C₂B₉H₁₂ (890 mg, 4.04 mmol) was dissolved in dry THF (20 mL) and the solution was cooled to 0° C. Gaseous ammonia was bubbled through the solution for 5 minutes at 0° C. After the ammonia flow was stopped, the reaction mixture was stirred for further 30 minutes at 0° C. The mixture was warmed to room temperature and stirred for 15 minutes. Degassed H₂O (10 mL) was added and the solution was stirred for 10 minutes. The solvent was evaporated under reduced pressure.

Yield of 10-(NH₃CH₂CH₂OCH₂CH₂O)-nido-7,8-C₂B₉H₁₁: 770 mg (3.24 mmol, 80%) of a white solid.

### Characterization:

1H {¹¹B} NMR (400.3 MHZ, CD₃CN): δ=6.72 (s, 3H, NH₃), 3.67 (m, 4H, CH₂), 3.58 (m, 2H, CH₂), 3.08 (t, 2H, CH₂), 2.01 (s, 2H, BH), 1.66 (s, 2H, C_{cluster}H), 1.41 (s, 1H, BH), 1.27 (s, 2H, BH), 1.12 (s, 2H, BH), −0.56 (s, 1H, □-BH) ppm.

¹¹B{¹H} NMR (128.4 MHZ, CD₃CN): δ=−10.51 (s, 1B), −12.56 (s, 2B), −17.36 (s, 2B), −23.66 (s, 2B), −24.88 (s, 1H), −40.61 (s, 1B) ppm.

¹¹B NMR (128.4 MHZ, CD₃CN): δ=−10.40 (s, 1B), −12.50 (d, 2B, ¹J (¹¹B,¹H)=136 Hz), −17.27 (d, 2B, ¹J (¹¹B,¹H)=132 Hz), −23.56 (d, 2B, ¹J (¹¹B,¹H)=149 Hz), −24.79 (d, 1B, ¹J (¹¹B,¹H)=161 Hz), −40.49 (d, 1B, ¹J (¹¹B,¹H)=141 Hz) ppm.

### 1.1.8 Syntheses of 7-Ph-10-O(CH₂)₄O-nido-7,8-C₂B9H₁₀

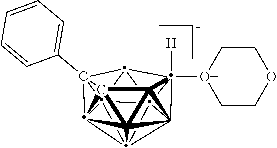

K[7-Ph-nido-7,8-C₂B₉H₁₀] (501 mg, 2.02 mmol) was suspended in dry toluene (6 mL) and sulfuric acid (0.9 mL) was added. The two-phase system was stirred vigorously for one hour. The toluene layer was separated and the sulfuric acid phase was extracted with toluene (2×2 mL). Dioxane (537 μL, 6.27 mmol) was added to the combined toluene phases. The clear solution was stirred 16 hours at 80° C. Afterwards the solvent was evaporated under reduced pressure. The residue was dissolved in CHCl₃ and was filtered through a short silica column. The product was eluted with CHCl₃ and then with CH₂Cl₂.

Yield: 451 mg (1.52 mmol, 75%) of a white solid.

¹¹B NMR (192.6 MHZ, CD₂Cl₂): δ=−8.6 (s, 1B), −10.8 (d, 1B, ¹J (¹¹B,¹H)=158.9 Hz), −12.1 (d, 1B, ¹J (¹¹B,¹H)=160.2 Hz), −17.0 (d, 1B, ¹J (¹¹B,¹H)=117.8 Hz), −18.0 (d, 2B, ¹J (¹¹B,¹H)=137.8 Hz), −19.5 (d, 1B, ¹J (¹¹B,¹H)=175.2 Hz), −22.1 (d, 1B, 1J (¹¹B,¹H)=155.6), −37.7 (d, 1B, ¹J (¹¹B,¹H)=143.5 Hz) ppm.

¹¹B{¹H} NMR (192.6 MHZ, CD₂Cl₂): δ=8.6 (s, 1B), −10.8 (s, 1B), −12.1 (s, 1B), −17.0 (s, 1B), −18.0 (s, 2B), −19.5 (s, 1B), −22.1 (s, 1B), −37.7 (s, 1B) ppm.

¹H NMR (600.2 MHZ, CD₂Cl₂): δ=7.28-7.10 (m, 5H, C_{Ar}H), 4.53 (t, 4H, CH₂), 3.94 (t, 4H, CH₂), 2.49 (s, 1H, C_{Cluster}H) ppm.

¹H {¹¹B} NMR (600.2 MHZ, CD₂Cl₂): δ=7.28-7.10 (m, 5H, CATH), 4.53 (t, 4H, CH₂), 3.94 (t, 4H, CH₂), 2.59 (s, 1H, BH), 2.55 (s, 1H, BH), 2.49 (m, 1H, C_{Cluster}H) 1.98 (s, 1H, BH), 1.83 (s, 1H, BH), 1.72 (s, 2H, BH), 1.48 (s, 1H, BH), 0.67 (s, 1H, BH) ppm; The signal of the bridging endo-proton of the boron cluster was not observed.

¹³C{¹H} NMR (150.9 MHZ, CD₂Cl₂): δ=143.2 (s, 1C, C_{Ar}), 128.2 (s, 2C, C_{Ar}), 127.2 (s, 2C, C_{Ar}), 126.3 (s, 1C, C_{Ar}), 82.5 (s, 4C, CH₂), 64.0 (m, 1C, C_{Cluster}), 65.3 (s, 4C, CH₂), 47.0 (m, 1C, HC_{Cluster}) ppm.

The 1,4-dioxane derivative 7-Ph-10-O(CH₂)₄O-nido-7,8-C₂B₉H₁₀ can be converted into the respective ammonia derivative according to Example 1.1. The ammonia derivative can be attached to polymer particles according to the procedure described under 1.8.12. Alternatively, the 1,4-dioxane derivative 7-Ph-10-O(CH2)₄O-nido-7,8-C2B9H10 can be converted into the respective allylamine derivative according to Example 1.4.

### 1.1.9 Syntheses of 7-Ph-10-O(CH₂)₂O(CH₂)₂NH₃-nido-7,8-C₂B₉H₁₀

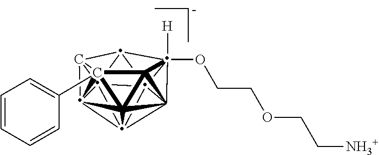

7-Ph-10-O(CH₂)₄O-nido-7,8-C₂B₉H₁₀ (599 mg, 2.02 mmol) was dissolved in dry THF (15 mL) and the solution was cooled to 0° C. Gaseous ammonia was bubbled though the solution. After five minutes, the gas flow was stopped, and the cloudy white suspension was slowly warmed to room temperature over 20 minutes. Degassed water (5 mL) was added and the solvent of the resulting yellow solution was evaporated under reduced pressure.

Yield: 480 mg (1.53 mmol, 76%).

¹¹B NMR (192.6 MHZ, CD₃CN): δ=−9.9 (s 1B), −10.4 (d, 1B, ¹J (¹¹B,¹H)=not resolved), −12.0 (d, 1B, ¹J (¹¹B,¹H)=134.7 Hz), −17.2 (d, 1B, 1J (¹¹B,¹H)=136.4 Hz), −18.5 (d, 1B, ¹J (¹¹B,¹H)=135.6 Hz), −21.4 (m, 2B, 1 J (¹¹B,¹H)=not resolved), −24.1 (d, 1B, ¹J (¹¹B,¹H)=149.4 Hz), −38.6 (d, 1B, 1J (¹¹B,¹H)=140.4 Hz) ppm.

¹¹B{¹H} NMR (192.6 MHZ, CD₃CN): δ=−9.9 (s 1B), −10.4 (s, 1B), −12.0 (s, 1B), −17.2 (s, 1B), −18.5 (s, 1B), −21.4 (m, 2B), −24.1 (s, 1B), −38.6 (s, 1B) ppm. 1H NMR (600.2 MHZ, CD₃CN): δ=7.20-7.16 (m, 2H, C_{Ar}Hₒᵣₜₕₒ), 7.16-7.11 (m, 2H, C_{Ar}Hₘₑₜₐ), 7.07-7.03 (m, 2H, C_{Ar}Hₚₐᵣₐ), 8.64 (s, 3H NH₃⁺), 3.71-3.62 (m, 4H, CH₂), 3.60-3.57 (m, 2H, CH₂), 3.12-3.04 (m, 2H, CH₂), 2.15 (s, 1H, C_{Cluster}H) ppm.

¹H {¹¹B} NMR (600.2 MHZ, CD₃CN): δ=7.20-7.16 (m, 2H, C_{Ar}Hₒᵣₜₕₒ), 7.16-7.11 (m, 2H, C_{Ar}Hₘₑₜₐ), 7.07-7.03 (m, 1H, C_{Ar}Hₚₐᵣₐ), 8.64 (s, 3H NH₃+), 3.71-3.62 (m, 4H, CH₂), 3.60-3.57 (m, 2H, CH₂), 3.12-3.04 (m, 2H, CH₂), 2.30 (s, 1H, BH), 2.52 (s, 1H, BH), 2.15 (m, 1H, C_{Cluster}H), 1.71 (s, 1H, BH), 1.49 (s, 1H BH), 1.38 (s, 1H BH), 1.34 (s, 1H, BH), 1.19 (s, 1H BH), 0.54 (s, 1H, BH), −0.08 (s, 1H, BH) ppm.

¹³C{¹H} NMR (150.9 MHZ, CD₃CN): δ=146.0 (s, 1C, C_{Ar}), 128.5 (s, 2C, C_{Ar}), 128.0 (s, 2C, C_{Ar}), 126.0 (s, 1C, C_{Ar}), 71.9 (s, 1C, CH₂), 71.0 (s, 1C, CH₂), 66.9 (s, 1C, CH₂), 60.4 (m, 1C, HC_{Cluster}), 43.6 (m, 1C, C_{Cluster}), 40.6 (s, 1C, CH₂) ppm.

The ammonia derivative can be attached to the polymer particles according to the procedure described under 1.8.12.

### 1.1.10 Syntheses of 7-Ph-10-O(CH₂)₂O(CH₂)₂NH₃-nido-7,8-C₂B₉H₁₀

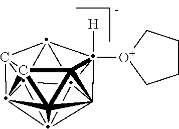

Na[nido-7,8-C₂B₉H₁₂] (709 mg, 4.53 mmol) was suspended in dry toluene (7.5 mL) and sulfuric acid (1.8 mL) was added. The two-phase system was stirred vigorously for 45 minutes at room temperature. The toluene layer was separated and the sulfuric acid phase was extracted with toluene (2×4 mL). Dry THF (678 mg, 9.40 mmol) was added to the combined toluene phases. The clear solution was stirred 21 hours at 80° C. The solvent was evaporated under reduced pressure. The residue was dissolved in CHCl₃ and filtered through a short silica column. The product was eluted with CHCl₃ and then with CH₂Cl₂.

Yield: 601 mg (2.94 mmol, 65%).

¹¹B NMR (160.5 MHZ, CD₂Cl₂): δ=−11.2 (s, 1B), −12.5 (d, 2B,

¹J (¹¹B,¹H)=144.1 Hz), −17.0 (s, 2B, ¹J (¹¹B,¹H)=137.6 Hz), −22.1 (s, 3B,

¹J (¹¹B,¹H)=151.1 Hz), −39.6 (s, 1B, ¹J (¹¹B,¹H)=144.0) ppm.

¹¹B{¹H} NMR (160.5 MHZ, CD₂Cl₂): δ==−11.2 (s, 1B), −12.5 (s, 2B), −17.0 (s, 2B), 22.07 (s, 3B), −39.6 (s, 1B) ppm.

¹H NMR (500.1 MHZ, CD₂Cl₂): δ=4.50-4.44 (m, 4H, CH₂), 2.23-2.18 (m, 4H, CH₂), 1.97 (s, 2H, C_{Cluster}H) ppm.

¹H {¹¹B} NMR (500.1 MHZ, CD₂Cl₂): δ=4.50-4.44 (m, 4H, CH₂), 2.23-2.18 (m, 4H, CH₂), 2.35 (s, 2H, BH), 1.97 (s, 2H, C_{Cluster}H), 1.65 (s, 1B, BH), 1.59 (s, 2B, BH), 1.34 (s, 2H, BH), 0.61 (s, 1H, BH), 0.12 (s, 1H, BH) ppm.

¹³C{¹H} NMR (125.8 MHZ, CD₂Cl₂): δ=83.2 (s, 2C, OCH₂), 42.0 (m, 2C, H_{CCluste}), 25.7 (s, 2C, CH₂) ppm.

The THF derivative 10-O(CH₂)₄-nido-7,8-C₂B₉H₁₁ can be reacted with ammonia to yield 10-O(CH₂)₄NH₃-nido-7,8-C₂B₉H₁₁ according to Example 1.1.2. The ammonia derivative can be attached to polymer particles according to the procedure described under 1.8.12. Alternatively, the THF derivative 10-O(CH₂)₄-nido-7,8-C₂B₉H₁₁ can be converted into an allylamine derivative according to Example 1.4.

### 1.2 Syntheses of acrylamides and N-allylamides with an adamantyl Substituent or with boron Clusters as Substituents

### 1.2.1 Synthesis of N-adamantylacrylamide

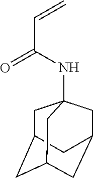

The synthesis was carried out according to a protocol described in the literature (Sokolov, V. B.; Aksinenko, A. Y.; Epishina, T. A.; Goreva, T. V.; Bachurin, S. O. Synthetic approaches to conjugation of aminoadamantanes and carbazoles. Russ. Chem. Bull. 2017, 66, 2110-2114; Bayguzina, A. R.; Lutfullina, A. R.; Khusnutdinov, R. I. Synthesis of N-(Adamantan-1-yl) carbamides by Ritter Reaction from Adamantan-1-ol and Nitriles in the Presence of Cu-Catalysts. Russ. J. Org. Chem. 2018, 54, 1127-1133.).

Amantadine (2.50 g, 16.5 mmol) and triethylamine (5.03 g, 6.70 mL, 49.6 mmol) were dissolved in dichloromethane (100 mL) and cooled to 0° C. Subsequently, acrylic acid chloride (1.79 g, 1.60 mL, 19.8 mmol) was added dropwise. The reaction mixture was stirred for 4 hours at room temperature and the solvent was removed under reduced pressure. The crude product was purified by column chromatography (eluent: diethyl ether/petroleum ether, 4:1+1% of triethylamine). Yield: 2.10 g (10.2 mmol, 62%), white solid.

### Characterization:

¹H NMR (400.4 MHZ, CDCl₃): δ=6.22 (dd, 1H, ³J_{HH}=16.9 Hz, ²J_{HH}=1.7 Hz, Hₜᵣₐₙₛ), 6.03 (dd, 1H, ³J_{HH}=16.9 Hz, ³J_{HH}=10.0 Hz, H_{gem}), 5.54 (dd, 1H, ³J_{HH}=10.0 Hz, ²J_{HH}=1.7 Hz, Hₒᵢₛ), 5.50 (s, 1H, NH), 2.09 (m, 9H, C₁₀H₁₅), 1.74 (m, 6H, C₁₀H₁₅) ppm.

### 1.2.2 Synthesis of 1-H₅C₃HNC(O)—C₁₀H₁₅

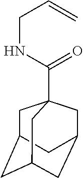 [SMILES: C=CCNC(=O)C12CC3CC(CC(C3)C1)C2]

1-Adamantanecarboxylic acid (2.00 g, 11.1 mmol) was dissolved in thionyl chloride (50 mL) and heated to 60° C. for 12 hours. Subsequently, all volatiles were removed under reduced pressure and the residue was dissolved in dichloromethane (50 mL). Allylamine (1.99 g, 2.50 mL, 33.3 mmol) was added dropwise at 0° C. The reaction mixture was stirred for 12 hours at room temperature and the solvent was removed under reduced pressure. The crude product was purified by column chromatography (eluent: hexane/ethyl acetate, 2:1). Yield: 1.71 g (7.80 mmol, 70%), white solid.

### Characterization:

1H NMR (400.4 MHZ, CDCl₃): δ=5.83 (ddt, 1H, ³J_{HH}=17.1 Hz, ³J_{HH}=10.2 Hz, ³J_{HH}=6.1 Hz, H_{gem}), 5.7 (s, 1H, NH), 5.14 (dd, 1H, ³J_{HH}=17.1 Hz, 2 J_{HH}=1.6 Hz, Hₜᵣₐₙₛ), 5.10 (dd, 1H, ³J_{HH}=10.4 Hz, ²J_{HH}=1.3 Hz, Hₒᵢₛ), 3.85 (tt, 2H, CH₂, 3J_{HH}=5.7 Hz, 4 J_{HH}=1.5 Hz), 2.03 (m, 3H, C₁₀H₁₅), 1.85 (m, 6H, C₁₀H₁₅), 1.71 (m, 6H, C₁₀H₁₅) ppm.

### 1.2.3 Synthesis of 1-H₃C₂C(O)HN-closo-1,2-C₂B₁₀H₁₁

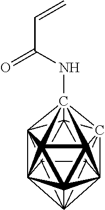

1-H₂N-closo-1,2-C₂B₁₀H₁₁ (2.25 g, 14.1 mmol) was dissolved in diethyl ether (200 mL) and mixed with triethylamine (8.68 g, 9.26 mL, 31.1 mmol). The solution was cooled to 0° C. and acrylic acid chloride (2.31 g, 2.08 mL, 16.9 mmol) was added dropwise. The reaction mixture was stirred for 12 hours at room temperature and the solvent was evaporated under reduced pressure. The crude product was purified by column chromatography (eluent: acetic acid ethyl ester/hexane, 1:1+1% of triethylamine). Yield: 1.05 g (4.92 mmol, 35%), white solid.

### Characterization:

1H NMR (500.1 MHZ, CDCl₃): δ=6.56 (s, 1H, NH), 6.37 (dd, 1H, ³J_{HH}=16.8 Hz, 2 J_{HH}=1.0 Hz, Hₜᵣₐₙₛ), 5.99 (dd, 1H, ³J_{HH}=16.8 Hz, ³J_{HH}=10.3 Hz, H_{gem}), 5.82 (dd, 1H, ³J_{HH}=10.3 Hz, ²J_{HH}=1.0 Hz, H_{cis}), 3.0-1.5 (m, 10H, BH), 1.27 (s, 1H, C_{Cluster}H) ppm.

¹H {¹¹B} NMR (500.1 MHZ, CDCl₃): δ=6.56 (s, 1H, NH), 6.37 (dd, 1H, 3J_{HH}=16.8 Hz, ²J_{HH}=1.0 Hz, Hₜᵣₐₙₛ), 5.99 (dd, 1H, 3J_{HH}=16.8 Hz, ³J_{HH}=10.3 Hz, H_{gem}), 5.82 (dd, 1H, ³J_{HH}=10.3 Hz, ²J_{HH}=1.0 Hz, H_{cis}), 2.39 (s, 2H, BH), 2.26 (s, 2H, BH), 2.20 (s, 6H, BH), 1.27 (s, 1H, C_{Cluster}H) ppm.

¹¹B NMR (160.4 MHZ, CDCl₃): δ=−4.3 (d, 1B, ¹J_{BH}=149 Hz), −7.1 (d, 1B, ¹J_{BH}=145 Hz), −11.1 (d, 6B, ¹J_{BH}=146 Hz), −13.8 (d, 2B, ¹J_{BH}=158 Hz) ppm. 11B {1H} NMR (160.4 MHZ, CDCl₃): δ=−4.3 (s, 1B), −7.1 (s, 1B), −11.1 (s, 6B), −13.8 (s, 2B) ppm.

¹³C{1H} NMR (125.7 MHZ, CDCl₃): δ=163.4 (s, 1C), 130.5 (s, 1C), 128.9 (s, 1C), 78.3 (s, 1C, C_{Cluster}N), 59.5 (s, 1C, C_{Cluster}H) ppm.

IR (ATR): [symbol] =3210 (w, w (N—H)), 3031 (m, v (═CH)), 2575 (s, v(B—H)), 1668 (m, v(C═O)), 1537 (m, w (C═C)) cm⁻¹.

### Elemental Analysis:

Calculated for 1-H₃C₂C(O)HN-closo-1,2-C₂B₁₀H₁₁: C, 28.16; H, 7.09; N, 6.57%. Found for C₅H₁₅B₁₀NO: C, 30.40; H, 7.24; N, 6.52%.

### 1.2.4 Synthesis of 1-H₅C₃HNC(O)-closo-1,7-C₂B₁₀H₁₁

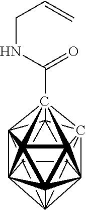

1-HO(O)C-closo-1,7-C₂B₁₀H₁₁ (2.70 g, 14.3 mmol) was dissolved in chloroform (150 mL) and mixed with thionyl chloride (2.00 ml, 3.41 g, 28.7 mmol). The reaction mixture was stirred for 1 hour at room temperature, and cooled down to 0° C. Subsequently, allylamine (2.37 mmol, 1.80 g, 31.6 mmol) was added. The reaction mixture was stirred for another 12 hours at room temperature. The solvent was removed under reduced pressure and the residue was purified by column chromatography (eluent: acetic acid ethyl ester/hexane, 1:1+1% of triethylamine). Yield: 1.31 g (5.76 mmol, 40%) of a beige solid.

### Characterization:

¹H NMR (500.1 MHZ, CDCl₃): δ=5.89 (s, 1H, NH), 5.78 (ddt, 1H, ³J_{HH}=17.0 Hz, ³J_{HH}=10.4 HZ, ³J_{HH}=5.4 Hz, H_{gem}), 5.15 (dd, 1H, ³J_{HH}=10.4 Hz, ²J_{HH}=1.6 Hz, H_{cis}), 5.14 (dd, 1H, ³J_{HH}=17.0 Hz, ²J_{HH}=1.6 Hz, Hₜᵣₐₙₛ), 3.82 (tt, 2H, CH₂ ³J_{HH}=5.4 Hz, 4 J_{HH}=1.6 Hz), 2.7-1.7 (m, 10H, BH), 1.25 (s, 1H, C_{Cluster}H) ppm.

¹H {¹¹B} NMR (500.1 MHZ, CDCl₃): δ=5.89 (s, 1H, NH), 5.78 (ddt, 1H, ³J_{HH}=17.0 Hz, ³J_{HH}=10.4 Hz, ³J_{HH}=5.4 Hz, H_{gem}), 5.15 (dd, 1H, 3J_{HH}=10.4 Hz, ²J_{HH}=1.6 Hz, H_{cis}), 5.14 (dd, 1H, ³J_{HH}=17.0 Hz, ²J_{HH}=1.6 Hz, Hₜᵣₐₙₛ), 3.82 (tt, 2H, CH₂, 3 J_{HH}=5.4 Hz, 4 J_{HH}=1.6 Hz), 3.04 (s, 2H, BH), 2.53 (s, 1H, BH), 2.45 (s, 2H, BH), 2.35 (s, 1H, BH), 2.28 (s, 2H, BH), 2.21 (s, 2H, BH), 1.25 (s, 1H, C_{Cluster}H) ppm.

¹¹B NMR (160.4 MHZ, CDCl₃): δ=−5.9 (d, 1B, ¹J_{BH}=162 Hz), −7.5 (d, 1B, ‘J_{BH}=162 Hz), −10.9 (d, 2B, ¹J_{BH}=141 Hz), −11.6 (d, 2B, ¹J_{BH}=139 Hz), −13.2 (d, 2B, 1 J_{BH}=166 Hz), −15.6 (d, 2B, ¹J_{BH}=164 Hz) ppm.

¹¹B {1H} NMR (160.4 MHZ, CDCl₃): δ=−5.9 (s, 1B), −7.5 (s, 1B), −10.9 (s, 2B), −11.6 (s, 2B), −13.2 (s, 2B), −15.6 (s, 2B) ppm.

¹³C{1H} NMR (125.7 MHz, CDCl₃): δ=160.0 (s, 1C), 132.7 (s, 1C), 117.0 (s, 1C), 74.2 (s, 1C, C_{Cluster}N), 54.9 (s, 1C, C_{Cluster}H), 42.9 (s, 1C) ppm.

IR (ATR): [symbol] =3378 (w, v(N—H)), 3014 (m, v(═CH)), 2925 (s, v(C—H)), 2604 (s, v(B—H)), 1661 (m, v(C═O)), 1517 (m, v(C═C)) cm⁻¹.

### Elemental Analysis:

Calculated for 1-H₅C₃HNC(O)-closo-1,7-C₂B₁₀H₁₁: C, 31.70; H, 7.54; N, 6.16%. Found for C₆H₁₇B₁₀NO: C, 33.04; H, 6.86; N, 5.41%.

### 1.2.5 Synthesis of 1-H₅C₃HNC(O)-closo-1,12-C₂B₁₀H₁₁

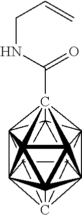

1-HO(O)C-closo-1,12-C₂B₁₀H₁₁ (0.50 g, 2.65 mmol) was dissolved in thionyl chloride (20 mL). The reaction mixture was stirred for 12 hours at 80° C. and subsequently all volatiles were removed under vacuum. The residue was dissolved in dichloromethane (20 mL) and mixed at 0° C. with allylamine (0.39 mL, 0.30 g, 5.3 mmol) and triethylamine (0.73 mL, 0.53 g, 5.3 mmol). The reaction mixture was stirred for another 12 hours at room temperature. The solvent was removed under reduced pressure and the residue was washed with water (2×30 mL). The residual solid was dried in a fine vacuum.

Yield: 0.53 g (2.33 mmol, 88%), white solid.

### Characterization:

¹H NMR (500.1 MHZ, CDCl₃): δ=5.71 (ddt, 1H, 3J_{HH}=17.2 Hz, ³J_{HH}=10.3 Hz, ³J_{HH}=5.2 Hz, H_{gem}), 5.67 (s, 1H, NH), 5.12 (dd, 1H, 3J_{HH}=10.2 Hz, ²J_{HH}=1.4 Hz, H_{cis}), 5.09 (dd, 1H, ³J_{HH}=17.2 Hz, 2 J_{HH}=1.4 Hz, Hₜᵣₐₙₛ), 3.82 (tt, 2H, CH₂, 3J_{HH}=5.6 Hz, ⁴J_{HH}=1.6 Hz), 2.80-1.97 (m, 10H, BH), 1.25 (s, 1H, C_{Cluster}H) ppm.

¹H {¹¹B} NMR (500.1 MHZ, CDCl₃): δ=5.71 (ddt, 1H, ³J_{HH}=17.2 Hz, ³J_{HH}=10.3 Hz, ³J_{HH}=5.2 Hz, H_{gem}), 5.67 (s, 1H, NH), 5.12 (dd, 1H, 3J_{HH}=10.2 Hz, ²J_{HH}=1.4 Hz, Hₒᵢₛ), 5.09 (dd, 1H, ³J_{HH}=17.2 Hz, ²J_{HH}=1.4 Hz, Hₜᵣₐₙₛ), 3.75 (tt, 2H, CH₂, 3 J_{HH}=5.6 Hz, ⁴J_{HH}=1.6 Hz), 2.45 (s, 5H, BH), 2.28 (s, 5H, BH), 1.25 (s, 1H, C_{Cluster}H) ppm.

¹¹B NMR (160.4 MHZ, CDCl₃): δ=−13.8 (d, 5B, ¹J_{BH}=169 Hz), −15.4 (d, 5B, ¹J_{BH}=170 Hz) ppm.

¹¹B{¹H} NMR (160.4 MHZ, CDCl₃): δ=−13.8 (s, 5B), −15.4 (s, 5B) ppm.

¹³C{1H} NMR (125.7 MHz, CDCl₃): δ=160.5 (s, 1C), 132.8 (s, 1C), 116.7 (s, 1C), 82.4 (s, 1C, C_{Cluster}N), 61.4 (s, 1C, C_{Cluster}H), 42.8 (s, 1C) ppm.

IR (ATR): [symbol] =3330 (w, v (N—H)), 3069 (m, v (═CH)), 2929 (s, (C—H)), 2607 (s,

v(B—H)), 1666 (m, v (C═O)), 1519 (m, ((C═C)) cm⁻¹.

### Elemental Analysis:

Calculated for 1-H₅C₃HNC(O)-closo-1,12-C₂B₁₀H₁₁: C, 31.70; H, 7.54; N, 6.16%. Found for C₆H₁₇B₁₀NO: C, 29.35; H, 6.05; N, 4.34%.

### 1.2.6 Synthesis of K[1-H₃C₂C(O)HN-closo-CB₁₁H₁₁]

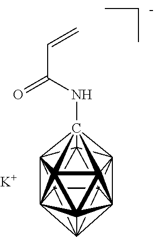

K[1-H₂N-closo-CB₁₁H₁₁] (3.00 g, 15.2 mmol) was dissolved in acetonitrile (50 mL) and triethylamine (4.61 g, 6.30 mL, 45.7 mmol) was added. The solution was cooled to 0° C. and acrylic acid chloride (1.65 g, 1.48 mL, 18.3 mmol) was added dropwise. The reaction mixture was stirred for 1 hour at room temperature and filtered. The filtrate was evaporated under reduced pressure and the residue was dissolved in 10% hydrochloric acid. The aqueous phase was extracted with diethyl ether (4×50 mL) and the organic phase was dried with potassium carbonate. The volume of the organic phase was reduced to ca. 30 mL. The addition of chloroform (100 mL) resulted in the precipitation of a white solid. It was separated by filtration and dried in a fine vacuum. Yield: 2.36 g (11.1 mmol, 73%).

### Characterization:

¹¹B{¹H} NMR (128.5 MHZ, CDCl₃): δ=−10.7 (s, 1B), −13.7 (s, 5B), −14.7 (s, 5B) ppm.

### 1.2.7 Synthesis of K[1-H₃C₂C(O)HN-12-I-closo-CB₁₁H₁₀]

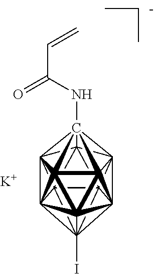

Cs[1-H₂N-12-I-closo-CB₁₁H₁₀] (500 mg, 1.19 mmol) was dissolved in acetonitrile (20 mL) and mixed with triethylamine (730 mg, 0.33 mL, 3.82 mmol). The solution was cooled to 0° C. and acrylic acid chloride (237 mg, 0.21 mL, 2.62 mmol) was added dropwise. The reaction mixture was stirred for 1 hour at room temperature and filtered. The filtrate was evaporated under reduced pressure and the residue was dissolved in 10% aqueous HCl. The aqueous phase was extracted with diethyl ether (4×50 mL) and organic phase was dried with potassium carbonate. After filtration, the volume of the organic phase was reduced to ca. 20 mL. The addition of chloroform (100 mL) resulted in the precipitation of a white solid. The product was separated by filtration and dried in a fine vacuum. Yield: 2.36 g 260 mg (0.69 mmol, 58%).

### Characterization:

1H NMR (500.1 MHZ, CDCl₃): δ=6.73 (s, 1H, NH), 6.10 (dd, 1H, ³J_{HH}=16.8 Hz, 2 J_{HH}=2.1 Hz, Hₜᵣₐₙₛ), 6.01 (dd, 1H, 3J_{HH}=16.8 Hz, ³J_{HH}=10.1 Hz, H_{gem}), 5.51 (dd, 1H, ³J_{HH}=10.1 Hz, ²J_{HH}=2.1 Hz, H_{cis}), 2.4-1.5 (m, 10H, BH) ppm. 1H {¹¹B} NMR (500.1 MHZ, CDCl₃): δ=6.73 (s, 1H, NH), 6.10 (dd, 1H, 3J_{HH}=16.8 Hz, ²J_{HH}=2.1 Hz, Hₜᵣₐₙₛ), 6.01 (dd, 1H, ³J_{HH}=16.8 Hz, ³J_{HH}=10.1 Hz, H_{gem}), 5.51 (dd, 1H, ³J_{HH}=10.1 Hz, ²J_{HH}=2.1 Hz, H_{cis}), 1.86 (s, 10H, BH) ppm.

¹¹B NMR (160.4 MHZ, CDCl₃): δ=−12.9 (d, 10B, ¹J_{BH}=137 Hz), −19.1 (s, 1B) ppm.

¹¹B{¹H} NMR (160.4 MHZ, CDCl₃): δ=−12.9 (s, 10B), −19.1 (s, 1B) ppm.

¹³C{¹H} NMR (125.7 MHz, CDCl₃): δ=163.4 (s, 1C), 130.5 (s, 1C), 125.5 (s, 1C), 74.8 (s, 1C, C_{Cluster}) ppm.

IR (ATR): [symbol] =3414 (w, (N—H)), 3031 (m, v (═CH)), 2552 (s, v(B—H)), 1600 (m, “(C═O)), 1489 (m, v (C═C)) cm⁻¹.

### Elemental Analysis:

Calculated for K[1-H₃C₂C(O)HN-12-I-closo-CB₁₁H₁₀]: C, 12.74; H, 3.74; N, 3.71%.

Found for C₄H₁₄B₁₁IKNO: C, 13.97; H, 3.81; N, 3.71%.

### 1.3 Derivatization of dioxane- and tetrahydrofuran-substituted closo-dodecaborate- and closo-decaborate Anions Using sodium allyl alcoholate

### General Protocol

The respective dioxane- and tetrahydrofuran-substituted closo-dodecaborate and closo-decaborate anions were dissolved in acetonitrile and mixed with potassium carbonate. The sodium allyl alcoholate was dissolved in acetonitrile and added to the suspension. The reaction mixture was stirred for 12 hours at 60° C. The suspension was filtered, and the solvent was removed under reduced pressure. The residue was dissolved in methanol and mixed with tetrabutylammonium hydroxide solution (1 M in methanol). The reaction mixture was evaporated under reduced pressure and the residue was taken up into dichloromethane. The organic phase was washed with water (3×50 mL) and dried over magnesium sulfate. After filtration, the solvent was removed under reduced pressure and the solid obtained was dried in a fine vacuum.

Metathesis to cesium salt:

The tetrabutylammonium salt was dissolved in dichloromethane and mixed with a solution of cesium fluoride in methanol. The precipitate was filtered off, washed with dichloromethane and methanol, and dried in a fine vacuum.

### 1.3.1 Synthesis of Kat₂[1-H₅C₃O(CH₂)₂O(CH₂)₂O-closo-B₁₂H₁₁] (Kat=[nBu₄N], Cs)

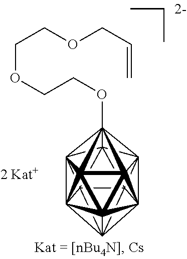

Quantities of Chemicals used for these Syntheses:

Kat=[nBu₄N]:
1.98 g (4.20 mmol) of [nBu₄N] [1-O(C₂H₄)₂O-closo-B₁₂H₁₁]; 5.80 g (42.0 mmol) of K₂CO₃;
0.67 g (8.40 mmol) of sodium allyl alcoholate;
50 mL of acetonitrile,
5.03 mL of a tetrabutylammonium hydroxide solution (1 M in methanol);
100 mL of dichloromethane.
Yield: 2.49 g (3.21 mmol, 77%), beige solid.

### Metathesis to Kat=Cs:

1.95 g (2.53 mmol) of [nBu₄N] 2 [1-H₅C₃O(CH₂)₂O(CH₂)₂O-closo-B₁₂H₁₁] dissolved in 20 mL of dichloromethane;
844 mg (5.55 mmol) of CsF dissolved in 30 mL of methanol.
Washing with 3×10 mL dichloromethane and 3×10 mL methanol.
Yield: 1.11 g (2.01 mmol, 80%), white solid.

### Characterization:

NMR data of the [1-H₅C₃O(CH₂)₂O(CH₂)₂O-closo-B₁₂H₁₁]²⁻ anion:

¹H NMR (500.1 MHZ, CD₃CN): δ=5.92 (ddt, 1H, 3J_{HH}=17.2 HZ, 3J_{HH}=10.4 Hz, ³J_{HH}=5.6 Hz, H_{gem}), 5.26 (dd, 1H, 3J_{HH}=17.2 Hz, ²J_{HH}=2.0 Hz, Hₜᵣₐₙₛ), 5.13 (dd, 1H, ³J_{HH}=10.4 Hz, ²J_{HH}=2.0 Hz, Hₒᵢₛ), 4.02 (dt, 2H, CH₂, 3 J_{HH}=5.6 Hz, 4 J_{HH}=1.5 Hz), 3.53-3.40 (m, 8H, 4CH₂), 1.45-0.41 (m, 11H, BH) ppm.

¹H {¹¹B} NMR (500.1 MHZ, CD₃CN): δ=5.92 (ddt, 1H, ³J_{HH}=17.2 Hz, ³J_{HH}=10.4 Hz, ³J_{HH}=5.6 Hz, H_{gem}), 5.26 (dd, 1H; 3J_{HH}=17.2 Hz, ²J_{HH}=2.0 Hz, Hₜᵣₐₙₛ), 5.13 (dd, 1H, ³J_{HH}=10.4 Hz, ²J_{HH}=2.0 Hz, H_{cis}), 4.02 (dt, 2H, CH₂, 3 J_{HH}=5.6 Hz, 4 J_{HH}=1.5 Hz, H₃), 3.53-3.40 (m, 8H, 4CH₂), 1.36 (s, 5H, BH), 0.99 (s, 5H, BH), 0.76 (s, 1H, BH) ppm.

¹¹B NMR (160.4 MHZ, CD₃CN): δ=6.4 (s, 1B), −16.7 (d, 5B, ¹J_{BH}=125 Hz), −18.1 (d, 5B, ¹J_{BH}=126 Hz), −22.9 (d, 1 J_{HH}=126 Hz, 1B) ppm.

¹¹B {1H} NMR (160.4 MHZ, CD₃CN): δ=6.4 (s, 1B), −16.6 (s, 5B), −18.1 (s, 5B), −22.9 (s, 1B) ppm.

¹³C{1H} NMR (125.7 MHZ, CD₃CN): δ=133.7 (s, 1C), 118.6 (s, 1C), 71.7 (s, 1C), 71.1 (s, 1C), 69.4 (s, 1C), 68.8 (s, 1C), 67.6 (s, 1C) ppm.

IR (ATR): [symbol] =3074 (vw, v(=CH)), 2959 (s, v(C—H)), 2870 (m, v(C—H)), 2480 (s, v(B—H)), 1474 (m, v(C═C)) cm⁻¹.

### Elemental Analysis:

Calculated for [nBu₄N]₂[1-H₅C₃O(CH₂)₂O(CH₂)₂O-closo-B₁₂H₁₁]: C, 60.76; H, 12.55; N, 3.63%.

Found for C₃₉H₉₆B₁₂N₂O₃: C, 58.49; H, 11.98; N, 3.62%.

Calculated for Cs₂[1-H₅C₃O(CH₂)₂O(CH₂)₂O-closo-B₁₂H₁₁]: C, 15.24; H, 4.38.

Found for C₇H₂₄B₁₂Cs₂O₃: C, 14.11; H, 4.13%.

### 1.3.2 Synthesis of Kat₂[1-H₅C₃O(CH₂)₄O-closo-B₁₂H₁₁] (Kat=[nBu₄N], Cs)

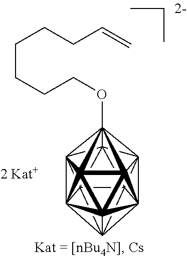

Quantities of Chemicals used for these Syntheses:

Kat=[nBu₄N]:
1.21 g (2.80 mmol) of [nBu₄N] [1-(C₂H₄)₄O-closo-B₁₂H₁₁]; 3.87 g (28.0 mmol) of K₂CO₃;
0.45 g (5.60 mmol) of sodium allyl alcoholate;
50 mL of acetonitrile,
6.02 mL of a tetrabutylammonium hydroxide solution (1 M in methanol);
60 mL of dichloromethane.
Yield: 1.49 g (1.97 mmol, 70%), beige solid.

### Metathesis to Kat=Cs:

1.34 g (1.78 mmol) of [nBu₄N]₂[1-H₅C₃O(CH₂)₄O-closo-B₁₂H₁₁] dissolved in 20 mL of dichloromethane;
594 mg (3.91 mmol) of CsF in 30 mL of methanol.
Washing with 3×10 mL dichloromethane and 3×10 mL methanol.
Yield: 460 mg (0.86 mmol, 48%), white solid.

### Characterization:

NMR data of the [1-H₅C₃O(CH₂)₄O-closo-B₁₂H₁₁]²⁻ anion:

¹H NMR (500.1 MHZ, CD₃CN): δ=5.92 (ddt, 1H, 3J_{HH}=17.3 HZ, ³J_{HH}=10.4 Hz, ³J_{HH}=5.3 Hz, H_{gem}), 5.26 (dd, 1H, ³J_{HH}=17.3 Hz, ²J_{HH}=1.8 Hz, Hₜᵣₐₙₛ), 5.11 (dd, 1H, ³J_{HH}=10.4 Hz, ²J_{HH}=1.8 Hz, Has), 4.06 (dt, 2H, CH₂, 3 J_{HH}=5.3 HZ, 4 J_{HH}=1.6 Hz), 3.60-3.53 (m, 4H, 2CH₂), 1.82-0.69 (m, 11H, BH), 1.60 (quin, 4H, 2CH₂, 3 J_{HH}=3.5 Hz) ppm.

¹H {¹¹B} NMR (500.1 MHZ, CD₃CN): δ=5.92 (ddt, 1H, ³J_{HH}=17.3 Hz, ³J_{HH}=10.4 Hz, ³J_{HH}=5.3 Hz, H_{gem}), 5.26 (dd, 1H, 3J_{HH}=17.3 Hz, ²J_{HH}=1.8 Hz, Hₜᵣₐₙₛ), 5.11 (dd, 1H, ³J_{HH}=10.4 Hz, ²J_{HH}=1.8 Hz, H_{cis}), 4.06 (dt, 2H, CH₂, 3J_{HH}=5.3 Hz, ⁴J_{HH}=1.6 Hz, H₃), 3.60-3.53 (m, 4H, 2CH₂), 1.60 (quin, 4H, 2CH₂, 3 J_{HH}=3.5 Hz), 1.34 (s, 5H, BH), 0.97 (s, 5H, BH), 0.74 (s, 1H, BH) ppm.

¹¹B{¹H} NMR (160.4 MHZ, CD₃CN): δ=6.5 (s, 1B), −16.4 (d, 5B, ¹J_{BH}=125 Hz), −18.1 (d, 5B, ¹J_{BH}=126 Hz), −23.1 (d, 1B, ¹J_{HH}=126 Hz) ppm.

¹¹B{¹H} NMR (160.4 MHZ, CD₃CN): δ=6.5 (s, 1B), −16.4 (s, 5B), −18.1 (s, 5B), −23.1 (s, 1B) ppm.

¹³C{¹H} NMR (125.7 MHZ, CDCl₃): δ=133.9 (s, 1C), 118.2 (s, 1C), 71.4 (s, 1C), 70.3 (s, 1C), 69.1 (s, 1C), 27.2 (s, 1C), 25.3 (s, 1C) ppm.

IR (ATR): [symbol] =3081 (vw, v(=CH)), 2938 (s, v(C—H)), 2872 (m, v(C—H)), 2463 (s, v(B—H)), 1477 (m, v(C═C)) cm⁻¹.

### Elemental Analysis:

Calculated for [nBu₄N]₂[1-H₅C₃O(CH₂)₄O-closo-B₁₂H₁₁]: C, 62.05; H, 12.82; N, 3.71%.

Found for C₃₉H₉₆B₁₂N₂O₂: C, 61.42; H, 12.63; N, 3.62%.

Calculated for Cs₂[1-H₅C₃O(CH₂)₄O-closo-B₁₂H₁₁]: C, 15.69; H, 4.52. Found for C₇H₂₄B₁₂Cs₂O₂: C, 14.84; H, 4.49%.

### 1.3.3 Synthesis of Kat₂[2-H₅C₃O(CH₂)₂O(CH₂)₂O-closo-B₁₀H₉] (Kat=[nBu₄N], Cs)

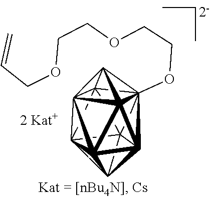

Quantities of Chemicals used for these Syntheses:

Kat=[nBu₄N]:
650 mg (1.45 mmol) of the [nBu₄N] [2-O(C₂H₄)₂O-closo-B₁₀H₉]; 449 mg (3.25 mmol) K₂CO₃;
157 mg (1.97 mmol) of the Sodium allyl alcoholate;
50 mL of acetonitrile,
2.05 mL of a tetrabutylammonium hydroxide solution (1 M in methanol);
60 mL of dichloromethane.
Yield: 690 mg (0.92 mmol, 64%), beige solid.

### Metathesis to Kat=Cs:

1.05 g (1.41 mmol) of the [nBu₄N] 2 [2-H₅C₃O(CH₂)₂O(CH₂)₂O-closo-B₁₀H₉] dissolved in 10 mL of dichloromethane;
470 mg (3.09 mmol) of CsF in 15 mL of methanol.
Washing with 3×5 mL dichloromethane and 3×5 mL methanol.
Yield: 212 mg (0.40 mmol, 28%), white solid.

### Characterization:

NMR data of the [2-H₅C₃O(CH₂)₂O(CH₂)₂O-closo-B₁₀H₉]²⁻ anion:

¹H NMR (500.1 MHZ, CD₃CN): δ=5.87 (ddt, 1H, ³J_{HH}=17.4 Hz, ³J_{HH}=10.5 Hz, ³J_{HH}=6.0 Hz, H_{gem}), 5.26 (dd, 1H, ³J_{HH}=17.3 Hz, ²J_{HH}=1.6 Hz, Hₜᵣₐₙₛ), 5.19 (dd,

¹H, ³J_{HH}=10.4 Hz, ²J_{HH}=1.4 Hz, Hₒᵢₛ), 3.98 (dt, 2H, CH₂, 3 J_{HH}=5.9 Hz, 4 J_{HH}=1.3 Hz), 3.58-3.30 (m, 8H, 4CH₂), 1.60-0.74 (m, 9H, BH) ppm.

¹H{¹¹B} NMR (500.1 MHZ, CD₃CN): δ=5.87 (ddt, 1H, ³J_{HH}=17.4 Hz, ³J_{HH}=10.5 Hz, ³J_{HH}=6.0 Hz, H_{gem}), 5.26 (dd, 1H, 3J_{HH}=17.3 Hz, ²J_{HH}=1.6 Hz, Hₜᵣₐₙₛ), 5.19 (dd, 1H, ³J_{HH}=10.4 Hz, ²J_{HH}=1.4 Hz, H_{cis}), 3.98 (dt, 2H, ³J_{HH}=5.9 Hz, 4 J_{HH}=1.3 Hz, H3), 3.58-3.30 (m, 8H, 4CH₂), 2.96 (s, 1H, BH), 0.87 (s, 2H, BH), 0.44 (s, 2H, BH), 0.12 (s, 2H, BH), −0.37 (s, 1H, BH) ppm.

¹¹B NMR (160.4 MHZ, CD₃CN): δ=−1.7 (s, 1B), −3.3 (d, 1B, ¹J_{BH}=147 Hz), −5.4 (d, 1B, ¹J_{BH}=142 Hz), −23.9 (d, 4B, ¹J_{BH}=145 Hz, B), −29.7 (d, 2B, ¹J_{BH}=132 Hz) −34.5 (d, 1B, ¹J_{BH}=136 Hz) ppm.

¹¹B{¹H} NMR (160.4 MHZ, CD₃CN): δ=−1.7 (s, 1B), −3.3 (s, 1B), −5.4 (s, 1B), −23.9 (s, 4B), −29.7 (s, 2B) −34.5 (s, 1B) ppm.

¹³C{¹H} NMR (125.7 MHZ, CD₃CN): δ=133.6 (s, 1° C.), 118.5 (s, 1° C.), 71.9 (s, 1C), 71.2 (s, 1C), 69.8 (s, 1C), 69.3 (s, 1C), 68.7 (s, 1C) ppm.

IR (ATR): [symbol] =3081 (vw, v(═CH)), 2958 (s, M (C—H)), 2872 (m, v (C—H)), 2438 (s, v(B—H)), 1473 (m, w (C═C)) cm⁻¹.

### Elemental Analysis:

Calculated for Cs₂[2-H₅C₃O(CH₂)₂O(CH₂)₂O-closo-B₁₀H₉]: C, 15.92; H, 4.20. Found for C₇H₂₄B₁₀Cs₂O₃: C, 14.35; H, 4.46%.

### 1.3.4 Synthesis of Kat₂[2-H₅C₃O(CH₂)₄O-closo-B₁₀H₉] (Kat=[nBu₄N], Cs)

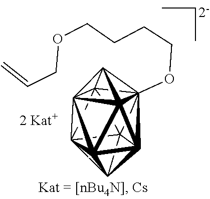

Quantities of Chemicals used for these Syntheses:

Kat=[nBu₄N]:
650 mg (1.51 mmol) of [nBu₄N] [2-(CH₂)₄O-closo-B₁₀Hg]; 1.04 g (7.55 mmol) of K₂CO₃;
266 mg (3.32 mmol) of sodium allyl alcoholate;
50 ml of acetonitrile;
6.02 mL of a tetrabutylammonium hydroxide solution (1 M in methanol);
60 mL of dichloromethane.
Yield: 1.08 g (1.48 mmol, 98%), beige solid.

### Metathesis to Kat=Cs:

940 mg (1.29 mmol) of [nBu₄N] 2 [2-H₅C₃O(CH₂)₄O-closo-B₁₀H₉] dissolved in 10 mL of dichloromethane;
427 mg (2.83 mmol) of CsF in 15 mL of methanol.
Washing with 3×5 mL dichloromethane and 3×5 mL methanol.
Yield: 377 mg (0.74 mmol, 57%), white solid.

### Characterization:

NMR data of the [2-H₅C₃O(CH₂)₄O-closo-B₁₀H₉]²⁻ anion:

¹H NMR (500.1 MHZ, CD₃CN): δ=5.87 (ddt, 1H, ³J_{HH}=17.3 Hz, ³J_{HH}=10.4 Hz, ³J_{HH}=6.0 Hz, H_{gem}), 5.26 (dd, 1H, ³J_{HH}=17.4 Hz, ²J_{HH}=2.3 Hz, Hₜᵣₐₙₛ), 5.19 (dd, 1H, ³J_{HH}=10.3 Hz, ²J_{HH}=1.7 Hz, Has), 3.96 (dt, 2H, CH₂, 3 J_{HH}=6.0 Hz, 4 J_{HH}=1.2 Hz), 3.44 (t, 2H, CH₂, 3 J_{HH}=6.2 Hz), 3.22 (t, 2H, CH₂, 3 J_{HH}=6.0 Hz), 1.60-0.74 (m, 9H, BH), 1.45-1.35 (m, 4H, 2CH₂) ppm.

¹H {¹¹B} NMR (500.1 MHZ, CD₃CN): δ=5.87 (ddt, 1H, 3J_{HH}=17.3 Hz, ³J_{HH}=10.4 Hz, ³J_{HH}=6.0 Hz, H_{gem}), 5.26 (dd, 1H, 3J_{HH}=17.4 Hz, ²J_{HH}=2.3 Hz, Hₜᵣₐₙₛ), 5.19 (dd, 1H, ³J_{HH}=10.3 Hz, ²J_{HH}=1.7 Hz, H_{cis}), 3.96 (dt, 2H, CH₂, 3 J_{HH}=6.0 Hz, ⁴J_{HH}=1.2 Hz), 3.44 (t, 2H, CH₂, 3 J_{HH}=6.2 Hz), 3.22 (t 2H, CH₂, 3J_{HH}=6.0 Hz), 2.96 (s. 1H, BH), 1.45-1.35 (m, 4H, 2CH₂), 0.87 (s, 2H, BH), 0.44 (s, 2H, BH), 0.12 (s, 2H, BH), −0.37 (s, 1H, BH) ppm.

¹¹B NMR (160.4 MHZ, CD₃CN): δ=−1.6 (s, 1B), −3.3 (d, 1B, ¹J_{BH}=147 Hz), −5.6 (d, 1B, ¹J_{BH}=142 Hz), −23.7 (d, 4B, ¹J_{BH}=145 Hz), −29.7 (d, 2B, ¹J_{BH}=132 Hz) −34.6 (d, 1B, ¹J_{BH}=136 Hz) ppm.

¹¹B {1H} NMR (160.4 MHZ, CD₃CN): δ=−1.6 (s, 1B), −3.3 (s, 1B), −5.6 (s, 1B), −23.7 (s, 4B), −29.7 (s, 2B) −34.6 (s, 1B) ppm.

¹³C{1H} NMR (125.7 MHz, CD₃CN): δ=133.9 (s, 1C), 118.2 (s, 1C), 71.3 (s, 1C), 71.2 (s, 1C), 70.3 (s, 1C), 27.2 (s, 1C), 25.2 (s, 1C) ppm.

IR (ATR): [symbol] =3085 (vw, v(=CH)), 2957 (s, v(CH)), 2871 (m, v(CH)), 2438 (s, v(BH)), 1478 (m, w (C═C)) cm⁻¹.

### Elemental Analysis:

Calculated for [nBu₄N] > [2-H₅C₃O(CH₂)₄O-closo-B₁₀H₉]: C, 64.05; H, 12.96; N, 3.83%.

Found for C₃₉H₉₄B₁₀N₂O₂: C, 62.53; H, 12.86; N, 3.85%.

Calculated for Cs₂[2-H₅C₃O(CH₂)₄O-closo-B₁₀H₉]: C, 16.42; H, 4.33. Found for C₇H₂₂B₁₀Cs₂O₂: C, 16.05; H, 4.46%.

### 1.4 Derivatization of dioxane- and tetrahydrofuran-substituted closo-dodecaborate- and closo-decaborate anions Using allylamine

General Protocol for the Reaction of dioxane and tetrahydrofuran Derivatives of closo-dodecaborate and closo-decaborate with allylamine

The reaction of dioxane and tetrahydrofuran derivatives of closo-dodecaborate and closo-decaborate anions with allylamine was carried out similar to the reaction with other amines as described in the literature (Semioshkin, A.; Nizhnik, E.; Godovikov, I.; Starikova, Z.; Bregadze, V. I. Reactions of oxonium derivatives of [B₁₂H₁₂]²⁻ with amines: Synthesis and structure of novel B12-based ammonium salts and amino acid. J. Organomet. Chem. 2007, 692, 4020-4028.). The respective derivative of the closo-dodecaborate or closo-decaborate anion was dissolved in allylamine and the reaction mixture was stirred for 12 hours at 60° C. The excess allylamine was distilled off under reduced pressure. The residue was dissolved in methanol and mixed with a tetrabutylammonium hydroxide solution (1 M in methanol). The solvent was removed under reduced pressure and the residue was taken up into dichloromethane. The organic phase was washed with water (3×50 mL) and dried with magnesium sulfate. The dichloromethane was distilled off under reduced pressure and the solid obtained was dried in a fine vacuum.

### 1.4.1 Synthesis of Kat₂[1-H₅C₃HN(CH₂)₂O(CH₂)₂O-closo-B₁₂H₁₁] (Kat=[nBu₄N], Cs)

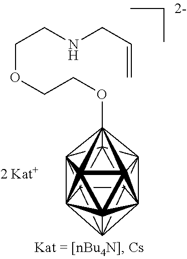

Quantities of Chemicals used for these Syntheses:

Kat=[nBu₄N]:
1.37 g (2.91 mmol) of [nBu₄N] [1-O(C₂H₄)₂O-closo-B₁₂H₁₁];
25 mL of allylamine
20 mL of methanol,
5.81 mL of a tetrabutylammonium hydroxide solution (1 M in methanol);
100 mL of dichloromethane.
Yield: 1.89 g (2.45 mmol, 85%), beige solid.

### Metathesis to Kat=Cs:

1.66 g (2.16 mmol) of [nBu₄N] 2 [1-H₅C₃HN(CH₂)₂O(CH₂)₂O-closo-B₁₂H₁₁] dissolved in 20 mL of dichloromethane;
679 mg (4.72 mmol) of CsF in 30 mL of methanol.
Washing with 3×10 mL dichloromethane and 3×10 mL methanol.
Yield: 1.00 g (1.82 mmol, 84%), white solid.

### Characterization:

NMR data of the [1-H₅C₃HN(CH₂)₂O(CH₂)₂O-closo-B₁₂H₁₁]²⁻ anion:

¹H NMR (500.1 MHZ, CD₃CN): δ=5.94 (ddt, 1H; ³J_{HH}=17.3 Hz, ³J_{HH}=10.4 Hz, ³J_{HH}=6.0 Hz, H_{gem}), 5.20 (dd, 1H, ³J_{HH}=17.3 Hz, ²J_{HH}=1.6 Hz, Hₜᵣₐₙₛ), 5.08 (dd, 1H, ³J_{HH}=10.4 Hz, ²J_{HH}=1.6 Hz, Has), 3.51 (t, 4H, 2CH₂, ³J_{HH}=5.4 Hz), 3.25 (dt, 2H, CH₂, 3J_{HH}=6.0 HZ, 4 J_{HH}=1.3 Hz), 2.70 (t, 4H, 2CH₂, 3 J_{HH}=5.4 Hz), 1.45-0.41 (m, 11H, BH) ppm, signal of NH was not observed.

¹H {¹¹B} NMR (500.1 MHZ, CD₃CN): δ=5.94 (ddt, 1H, 3J_{HH}=17.3 Hz, ³J_{HH}=10.4 Hz, ³J_{HH}=6.0 Hz, H_{gem}), 5.20 (dd, 1H, 3J_{HH}=17.3 Hz, ²J_{HH}=1.6 Hz, Hₜᵣₐₙₛ), 5.08 (dd, 1H, ³J_{HH}=10.4 Hz, ²J_{HH}=1.6 Hz, H_{cis}), 3.51 (t, 4H, 2CH₂, 3 J_{HH}=5.4 Hz), 3.25 (dt, 2H, CH₂, 3 J_{HH}=6.0 HZ, 4 J_{HH}=1.3 Hz), 2.70 (t, 4H, CH₂, 3 J_{HH}=5.4 Hz), 1.36 (s, 5H, BH), 0.99 (s, 5H, BH), 0.76 (s, 1H, BH) ppm, signal of NH was not observed.

¹¹B NMR (160.4 MHZ, CD₃CN): δ=6.4 (s, 1B, B1), −16.4 (d, 5B, ¹J_{BH}=125 Hz, B2-6), −18.2 (d, 5B, ¹J_{BH}=126 Hz, B7-11), −23.1 (d, 1B, ¹J_{HH}=126 Hz, B12) ppm.

¹¹B{¹H} NMR (160.4 MHZ, CD₃CN): δ=6.4 (s, 1B), −16.4 (s, 5B), −18.2 (s, 5B), −23.1 (s, 1B) ppm.

¹³C{1H} NMR (125.7 MHZ, CDCl₃): δ=136.8 (s, 1C), 115.5 (s, 1C), 72.7 (s, 1C), 69.5 (s, 1C), 67.9 (s, 1C), 51.5 (s, 1C), 48.3 (s, 1C) ppm.

IR (ATR): [symbol] =3294 (w, v(N—H)), 3074 (vw, v(═CH)), 2956 (s, v(C—H)), 2872 (m, v(C—H)), 2466 (s, v(B—H)), 1469 (m, v (C═C)) cm⁻¹.

### 1.4.2 Synthesis of Kat₂[1-H₅C₃HN(CH₂)₄O-closo-B₁₂H₁₁] (Kat=[nBu₄N], Cs)

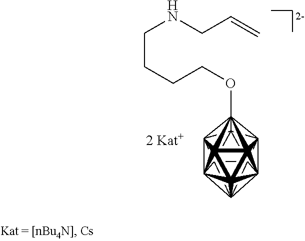

Quantities of Chemicals used for these Syntheses:

Kat=[nBu₄N]:
1.64 g (3.79 mmol) of [nBu₄N] [1-(CH₂)₄O-closo-B₁₂H₁₁];
25 mL of allylamine;
20 mL of methanol;
7.60 mL of a tetrabutylammonium hydroxide solution (1 M in methanol);
70 mL of dichloromethane.
Yield: 2.42 g (3.21 mmol, 85%), beige solid.

### Metathesis to Kat=Cs:

2.27 g (3.01 mmol) of [nBu₄N] 2 [1-H₅C₃HN(CH₂)₄O-closo-B₁₂H₁₁] dissolved in 20 mL of dichloromethane;
1.01 g (6.65 mmol) of CsF in 30 mL of methanol.
Washing with 3×10 mL dichloromethane and 3×10 mL methanol.
Yield: 0.79 g (1.48 mmol, 39%), white solid.

### Characterization:

NMR data of the [1-H₅C₃HN(CH₂)₄O-closo-B₁₂H₁₁]²⁻ anion:

¹H NMR (500.1 MHZ, CD₃CN): δ=5.93 (ddt, 1H, 3J_{HH}=17.4 Hz, ³J_{HH}=10.4 Hz, ³J_{HH}=6.0 Hz, H_{gem}), 5.19 (dd, 1H, ³J_{HH}=17.4 Hz, ²J_{HH}=1.7 Hz, Hₜᵣₐₙₛ), 5.02 (dd, 1H, ³J_{HH}=10.4 Hz, ²J_{HH}=1.7 Hz, Has), 3.36 (t, 2H, CH₂, ³J_{HH}=5.9 Hz), 3.24 (dt, 2H, CH₂, 3 J_{HH}=6.0 Hz, ⁴J_{HH}=1.4 Hz), 2.56 (t, 2H, CH₂, 3J_{HH}=5.9 Hz), 1.82-0.69 (m, 11H, BH), 1.45 (quin, 4H, 2CH₂, 3J_{HH}=3.5 Hz) ppm, signal of NH was not observed.

¹H NMR (500.1 MHZ, CD₃CN): δ=5.93 (ddt, 1H, ³J_{HH}=17.4 Hz, ³J_{HH}=10.4 Hz, ³J_{HH}=6.0 Hz, H_{gem}), 5.19 (dd, 1H, 3J_{HH}=17.4 Hz, 2 J_{HH}=1.7 Hz, Hₜᵣₐₙₛ), 5.02 (dd, 1H, ³J_{HH}=10.4 Hz, ²J_{HH}=1.7 Hz, Has), 3.36 (t, 2H, CH₂, 3 J_{HH}=5.9 Hz, H_{4/7}), 3.24 (dt, 2H, CH₂, 3 J_{HH}=6.0 HZ, 4 J_{HH}=1.4 Hz, H₃), 2.56 (t, 2H, CH₂, 3 J_{HH}=5.9 Hz, H_{4/7}), 1.45 (quin, 4H, 2CH₂, 3 J_{HH}=3.5 Hz, H_{5,6}), 1.14 (s, 5H, BH), 0.83 (s, 5H, BH), 0.58 (s, 1H, BH) ppm, signal of NH was not observed.

¹¹B NMR (160.4 MHZ, CD₃CN): δ=6.4 (s, 1B), −16.7 (d, 5B, ¹J_{BH}=125 Hz), −18.0 (d, 5B, ¹J_{BH}=126 Hz), −22.8 (d, 1B, ¹J_{HH}=126 Hz) ppm.

¹¹B{¹H} NMR (160.4 MHZ, CD₃CN): δ=6.4 (s, 1B), −16.7 (s, 5B), −18.0 (s, 5B), −22.8 (s, 1B) ppm.

¹³C{¹H} NMR (125.7 MHZ, CDCl₃): δ=136.6 (s, 1C), 115.7 (s, 1C), 68.4 (s, 1C), 51.5 (s, 1C), 48.8 (s, 1C), 28.3 (s, 1C), 24.3 (s, 1C) ppm.

IR (ATR): [symbol] =3151 (w, v(N—H)), 3077 (vw, v(═CH)), 2959 (s, v(C—H)), 2873 (m, v(C—H)), 2460 (s, v(B—H)), 1470 (m, v(C═C)) cm⁻¹.

### Elemental Analysis:

Calculated for [nBu₄N] 2 [1-H₅Ca HN(CH₂)₄O-closo-B₁₂H₁₁]: C, 62.13; H, 12.97; N, 5.57%.

Found for C₃₉H₉₇B₁₂NO: C, 60.59; H, 12.57; N, 5.12%.

Calculated for Cs₂[1-H₅C₃HN(CH₂)₄O-closo-B₁₂H₁₁]: C, 15.72; H, 4.71; N, 2.62%.

Found for C₇H₂₅B₁₂Cs₂NO: C, 16.29; H, 4.93; N, 2.28%.

### 1.4.3 Synthesis of Kat₂[2-H₅C3HN(CH₂)₂O(CH₂)₂O-closo-B₁₀H₉] (Kat=[nBu₄N], Cs)

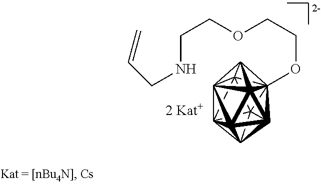

Quantities of Chemicals used for these Syntheses:

Kat=[nBu₄N]:
650 mg (1.45 mmol) of [nBu₄N] [2-O(C₂H₄)₂O-closo-B₁₀Hg];
25 mL of allylamine;
20 mL of methanol;
1.43 mL of a tetrabutylammonium hydroxide solution (1 M in methanol);
70 mL of dichloromethane.
Yield: 1.06 g (1.42 mmol, 98%), beige solid.

### Metathesis to Kat=Cs:

1.24 g (1.68 mmol) of [nBu₄N]₂[2-H₅C₃HN(CH₂)₂O(CH₂)₂O-closo-B₁₀H₉] dissolved in 20 mL of dichloromethane;
561 mg (3.98 mmol) of CsF in 30 mL of methanol.
Washing with 3×5 mL dichloromethane and 3×5 mL methanol.
Yield: 432 mg (0.82 mmol, 49%), white solid.

### Characterization:

NMR data of the [2-H₅C₃HN(CH₂)₂O(CH₂)₂O-closo-B₁₀H₉]²⁻ anion:

¹H NMR (500.1 MHZ, CD₃CN): δ=5.84 (ddt, 1H, ³J_{HH}=17.1 Hz, ³J_{HH}=10.2 Hz, ³J_{HH}=6.8 Hz, H_{gem}), 5.44 (dd, 1H, 3J_{HH}=17.1 Hz, ²J_{HH}=1.9 Hz, Hₜᵣₐₙₛ), 5.41 (dd, 1H, ³J_{HH}=10.0 Hz, ²J_{HH}=1.4 Hz, Has), 3.62 (t, 2H, CH₂, 3 J_{HH}=5.0 Hz), 3.59 (dt, 2H, CH₂, 3 J_{HH}=6.8 Hz, 4 J_{HH}=1.1 Hz), 3.45 (t, 2H, CH₂, 3J_{HH}=4.8 Hz), 3.35 (t, 2H, CH₂, 3 J_{HH}=4.9 Hz), 3.10 (t, 2H, CH₂, 3 J_{HH}=5.3 Hz), 1.60-0.74 (m, 9H, BH) ppm, signal of NH was not observed.

¹H{¹¹B} NMR (500.1 MHZ, CD₃CN): δ=5.84 (ddt, 1H, 3J_{HH}=17.1 HZ, ³J_{HH}=10.2 Hz, ³J_{HH}=6.8 Hz, H_{gem}), 5.44 (dd, 1H, 3J_{HH}=17.1 Hz, ²J_{HH}=1.9 Hz, Hₜᵣₐₙₛ), 5.41 (dd, 1H, ³J_{HH}=10.0 Hz, ²J_{HH}=1.4 Hz, H_{cis}), 3.62 (t, 2H, CH₂, 3 J_{HH}=5.0 Hz), 3.59 (dt, 2H, CH₂, 3 J_{HH}=6.8 Hz, 4 J_{HH}=1.1 Hz), 3.45 (t, 2H, CH₂, 3 J_{HH}=4.8 Hz), 3.35 (t, 2H, CH₂, 3J_{HH}=4.9 Hz), 3.10 (t, 2H, CH₂, 3 J_{HH}=5.3 Hz), 2.96 (s, 1H, BH), 0.87 (s, 2H, BH), 0.44 (s, 2H, BH), 0.12 (s, 2H, BH), −0.37 (s, 1H, BH) ppm, signal of NH was not observed.

¹¹B NMR (160.4 MHZ, CD₃CN): δ=−1.8 (s, 1B), −3.2 (d, 1B, ¹J_{BH}=147 Hz), −5.4 (d, 1B, ¹J_{BH}=142 Hz), −24.0 (d, 4B, ¹J_{BH}=145 Hz), −29.9 (d, 2B, ¹J_{BH}=132 Hz) −34.5 (d, 1B, ¹J_{BH}=136 Hz) ppm.

¹¹B {1H} NMR (160.4 MHZ, CD₃CN): δ=−1.8 (s, 1B), −3.2 (s, 1B), −5.4 (s, 1B), −24.0 (s, 4B), −29.9 (s, 2B) −34.5 (s, 1B) ppm.

¹³C{¹H} NMR (125.7 MHZ, CD₃CN): δ=127.8 (s, 1C), 123.4 (s, 1C), 71.2 (s, 1C), 70.0 (s, 1C), 65.1 (s, 1C), 49.3 (s, 1C), 46.1 (s, 1C).

IR (ATR): [symbol] =3208 (w, v(N—H)), 3066 (vw, v(═CH)), 2958 (s, M (C—H)), 2871 (m, v(C—H)), 2438 (s, v(B—H)), 1470 (m, w (C═C)) cm⁻¹.

### 1.4.4 Synthesis of Kat₂[2-H₅C₃HN(CH₂)₄O-closo-B₁₀H₉] (Kat=[nBu₄N], Cs)

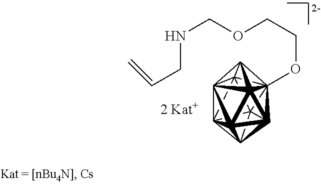

Quantities of Chemicals used for these Syntheses:

Kat=[nBu₄N]:
650 mg (1.51 mmol) of [nBu₄N] [2-(CH₂)₄O-closo-B₁₀H₉];
25 mL of allylamine,
20 mL of methanol,
1.43 mL of a tetrabutylammonium hydroxide solution (1 M in methanol);
70 mL of dichloromethane.
Yield: 1.10 g (1.50 mmol, 99%), beige solid.

### Metathesis to Kat=Cs:

569 mg (0.78 mmol) of [nBu₄N] 2 [2-H₅C₃HN(CH₂)₄O-closo-B₁₀H₉] dissolved in 20 mL of dichloromethane;
260 mg (1.71 mmol) of CsF in 30 mL of methanol.
Washing with 3×5 mL dichloromethane and 3×5 mL methanol.
Yield: 176 mg (0.34 mmol, 44%), white solid.

### Characterization:

NMR data of the [2-H₅C₃HN(CH₂)₄O-closo-B₁₀H₉]²⁻ anion:

¹H NMR (500.1 MHZ, CD₃CN): δ=5.86 (ddt, 1H, 3J_{HH}=17.2 Hz, ³J_{HH}=10.4 Hz, ³J_{HH}=6.7 Hz, H_{gem}), 5.43 (dd, 1H, ³J_{HH}=17.0 Hz, ²J_{HH}=1.2 Hz, Hₜᵣₐₙₛ), 5.40 (dd, 1H, ³J_{HH}=10.4 Hz, ²J_{HH}=1.2 Hz, Has), 3.57 (d, 2H, CH₂, 3J_{HH}=7.1 Hz), 3.20 (t, 2H, CH₂, 3 J_{HH}=6.2 Hz), 2.88 (t, 2H, CH₂, 3J_{HH}=6.7 Hz), 1.60-0.74 (m, 9H, BH), 1.55 (quin, 2H, CH₂, 3 J_{HH}=6.9 Hz), 1.45 (quin, 2H, CH₂, 3 J_{HH}=6.5 Hz) ppm, signal of NH was not observed.

¹H {¹¹B} NMR (500.1 MHZ, CD₃CN): δ=5.86 (ddt, 1H, 3J_{HH}=17.2 Hz, ³J_{HH}=10.4 Hz, ³J_{HH}=6.7 Hz, H_{gem}), 5.43 (dd, 1H, 3J_{HH}=17.0 Hz, ²J_{HH}=1.2 Hz, Hₜᵣₐₙₛ), 5.40 (dd, 1H,³J_{HH}=10.4 Hz, ²J_{HH}=1.2 Hz, H_{cis}), 3.57 (d, 2H, CH₂, 3 J_{HH}=7.1 Hz), 3.20 (t, 2H, CH₂, 3 J_{HH}=6.2 Hz), 2.96 (s, 1H, BH), 2.88 (t, 2H, CH₂, 3 J_{HH}=6.7 Hz), 1.55 (quin, 2H, CH₂, 3 J_{HH}=6.9 Hz), 1.45 (quin, 2H, CH₂, 3 J_{HH}=6.5 Hz), 0.87 (s, 2H, BH), 0.44 (s, 2H, BH), 0.12 (s, 2H, BH), −0.37 (s, 1H, BH) ppm, signal of NH was not observed.

¹¹B NMR (160.4 MHZ, CD₃CN): δ=−1.7 (s, 1B), −3.5 (d, 1B, ¹J_{BH}=147 Hz, 1B,), −5.5 (d, 1B, ¹J_{BH}=142 Hz), −23.8 (d, 4B, ¹J_{BH}=145 Hz), −29.5 (d, 2B, ¹J_{BH}=132 Hz) −34.4 (d, 1B, ¹J_{BH}=136 Hz) ppm.

¹¹B {1H} NMR (160.4 MHZ, CD₃CN): δ=−1.7 (s, 1B), −3.5 (s, 1B), −5.5 (s, 1B), −23.8 (s, 4B), −29.5 (s, 2B) −34.4 (s, 1B) ppm.

¹³C{1H} NMR (125.7 MHZ, CDCl₃): δ=127.9 (s, 1C), 123.3 (s, 1C), 70.9 (s, 1C), 49.3 (s, 1C), 46.7 (s, 1C), 28.2 (s, 1C), 23.4 (s, 1C) ppm.

IR (ATR): [symbol] =3083 (vw, v(═CH)), 2938 (s, v(C—H)), 2872 (m, v(C—H)), 2463 (s, v(B—H)), 1477 (m, v(C═C)) cm⁻¹.

### 1.5 Derivatization of dioxane- and tetrahydrofuran-substituted closo-dodecaborate- and closo-decaborate anions Using acrylic acid chloride

General Protocol for the Reaction of Amino-Functionalized closo-dodecaborate and closo-decaborate Derivatives with acrylic acid chloride:

The respective closo-dodecaborate or closo-decaborate derivative with an amino functionality was dissolved in acetonitrile and mixed with triethylamine. The solution was cooled to 0° C. and acrylic acid chloride was added dropwise. The reaction was stirred for 12 hours at room temperature, filtered, and the solvent was removed under reduced pressure. The residue was dissolved in dichloromethane and washed with 10% aqueous HCl and water. The organic phase was dried over magnesium sulfate, filtered, and evaporated under reduced pressure. The solid obtained was dried in a fine vacuum.

### 1.5.1 Synthesis of Kat₂[1-H₃C₂C(O)HN(CH₂)₂O(CH₂)₂O-closo-B₁₂H₁₁] (Kat= [nBu₄N], Cs)

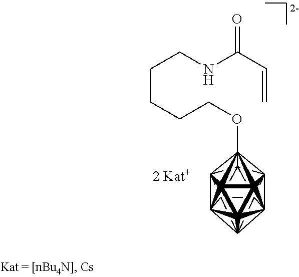

Quantities of Chemicals used for these Syntheses:

Kat=[nBu₄N]:
4.22 g (5.77 mmol) of [nBu₄N] 2 [1-H₂N(CH₂)₂O(CH₂)₂O-closo-B₁₂H₁₁];
2.37 mL (1.75 g, 17.3 mmol) of triethylamine;
0.71 ml (0.78 g, 8.66 mmol) of acrylic acid chloride;
20 mL of dichloromethane,
3×20 mL of an 10% aqueous HCl
2×20 ml of water.
Yield: 2.27 g (2.90 mmol, 50%), beige solid.

### Metathesis to Kat=Cs:

2.03 g (2.59 mmol) of [nBu₄N] 2 [1-H₃C₂C(O)HN(CH₂)₂O(CH₂)₂O-closo-B₁₂H₁₁] dissolved in 20 mL of dichloromethane;
863 mg (5.68 mmol) of CsF in 30 mL of methanol.
Washing with 3×15 mL dichloromethane and 3×15 mL methanol.
Yield: 1.32 g (2.33 mmol, 90%), white solid.

### Characterization:

NMR data of the [1-H₃C₂C(O)HN(CH₂)₂O(CH₂)₂O-closo-B₁₂H₁₁]²⁻ anion:

¹H NMR (500.1 MHZ, CD₃CN): δ=6.34 (dd, 1H, ³J_{HH}=17.0 Hz, ³J_{HH}=10.2 Hz, H_{gem}), 6.13 (dd, 1H, ³J_{HH}=17.0 HZ, ²J_{HH}=2.2 Hz, Hₜᵣₐₙₛ), 5.53 (dd, 1H, ³J_{HH}=10.2 Hz, ²J_{HH}=2.2 Hz, H_{cis}), 3.64-3.54 (m, 8H, 4CH₂), 1.72-0.60 (m, 11H, BH) ppm, signal of NH was not observed.

¹H {¹¹B} NMR (500.1 MHZ, CD₃CN): δ=6.34 (dd, 1H, ³J_{HH}=17.0 Hz, ³J_{HH}=10.2 Hz, H_{gem}), 6.13 (dd, 1H, 3J_{HH}=17.0 Hz, ²J_{HH}=2.2 Hz, Hₜᵣₐₙₛ), 5.53 (dd, 1H, ³J_{HH}=10.2 Hz, ²J_{HH}=2.2 Hz, H_{cis}), 3.64-3.54 (m, 8H, 4CH₂), 1.34 (s, 5H, BH), 0.97 (s, 5H, BH), 0.75 (s, 1H, BH) ppm, signal of NH was not observed.

¹¹B NMR (160.4 MHZ, CD₃CN): δ=6.2 (s, 1B), −16.7 (d, 5B, ¹J_{BH}=125 Hz), −17.8 (d, 5B, ¹J_{BH}=126 Hz), −22.6 (d, 1 J_{HH}=126 Hz, 1B) ppm.

¹¹B{¹H} NMR (160.4 MHZ, CD₃CN): δ=6.2 (s, 1B), −16.7 (s, 5B), −17.8 (s, 5B), −22.6 (s, 1B) ppm.

¹³C{¹H} NMR (125.7 MHZ, CDCl₃): δ=165.3 (s, 1C), 132.3 (s, 1C), 124.2 (s, 1C), 71.0 (s, 1C), 68.5 (s, 1C), 67.7 (s, 1C), 67.5 (s, 1C) ppm.

IR (ATR): [symbol] =3360 (w, v(N—H)), 3059 (vw, v(═CH)), 2959 (s, v(C—H)), 2872 (m, v(C—H)), 2463 (s, v(B—H)), 1666 (m, v(C═O)), 1469 (m, v(C═C)) cm⁻¹.

### Elemental Analysis:

Calculated for Cs₂[1-H₃C₂C(O)HN(CH₂)₂O(CH₂)₂O-closo-B₁₂H₁₁]: C, 14.89; H, 4.10; N, 2.48%.

Found for C₇H₂₃B₁₂Cs₂NO₃: C, 14.20; H, 5.33; N, 2.48%.

### 1.5.2 Synthesis of Kat₂[1-H₃C₂C(O)HN(CH₂)₄O-closo-B₁₂H₁₁] (Kat=[nBu₄N], Cs)

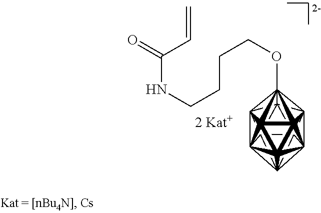

Quantities of Chemicals used for these Syntheses:

Kat=[nBu₄N]:
2.15 g (3.01 mmol) of the [nBu₄N] 2 [1-H₂N(CH₂)₄O-closo-B₁₂H₁₁];
1.24 mL (0.91 g, 9.03 mmol) of triethylamine;
0.37 ml (0.41 g, 4.52 mmol) of acrylic acid chloride;
20 mL of dichloromethane,
3× 20 mL of 10% aqueous HCl
2×20 ml of water.
Yield: 1.29 g (1.68 mmol, 56%), beige solid.

### Metathesis to Kat=Cs:

1.13 g (1.47 mmol) of [nBu₄N] 2 [1-H₃C₂C(O)HN(CH₂)₄O-closo-B₁₂H₁₁] dissolved in 20 mL of dichloromethane;
492 mg (3.24 mmol) of CsF in 30 mL of methanol.
Washing with 3×10 mL dichloromethane and 3×10 mL methanol.
Yield: 0.60 g (1.09 mmol, 74%), white solid.

### Characterization:

NMR data of the [1-H₃C₂C(O)HN(CH₂)₄O-closo-B₁₂H₁₁]²⁻ anion:

¹H NMR (500.1 MHZ, CD₃CN): δ=6.45 (dd, 1H, ³J_{HH}=17.1 Hz, ³J_{HH}=10.3 Hz, H_{gem}), 6.09 (dd, 1H, ³J_{HH}=17.1 Hz, ²J_{HH}=2.2 Hz, Hₜᵣₐₙₛ), 5.47 (dd, 1H, 3J_{HH}=10.3 Hz, ²J_{HH}=2.2 Hz, H_{cis}), 3.55 (1, 2H, CH₂, 3 J_{HH}=5.8 Hz), 3.30 (t, 2H, CH₂, 3 J_{HH}=6.1 Hz), 1.80-0.53 (m, 11H, BH), 1.58 (quin, 4H, 2CH₂, 3 J_{HH}=6.3 Hz) ppm, signal of NH was not observed.

¹H {¹¹B} NMR (500.1 MHZ, CD₃CN): δ=6.45 (dd, 1H, ³J_{HH}=17.1 Hz, ³J_{HH}=10.3 Hz, H_{gem}), 6.09 (dd, 1H, ³J_{HH}=17.1 Hz, ²J_{HH}=2.2 Hz, Hₜᵣₐₙₛ), 5.47 (dd, 1H, ³J_{HH}=10.3 Hz, ²J_{HH}=2.2 Hz, H_{cis}), 3.55 (t, 2H, CH₂, 3 J_{HH}=5.8 Hz), 3.30 (t, 2H, CH₂, 3 J_{HH}=6.1 Hz), 1.58 (quin, 4H, 2CH₂, 3 J_{HH}=6.3 Hz), 1.43 (s, 5H, BH), 1.06 (s, 5H, BH), 0.83 (s, 1H, BH) ppm, signal of NH was not observed.

¹¹B NMR (160.4 MHZ, CD₃CN): δ=6.3 (s, 1B), −16.6 (d, 5B, ¹J_{BH}=125 Hz), −18.0 (d, 5B, ¹J_{BH}=126 Hz), −22.7 (d, 1B, ¹J_{HH}=126 Hz) ppm.

¹¹B {1H} NMR (160.4 MHZ, CD₃CN): δ=6.3 (s, 1B), −16.6 (s, 5B), −18.0 (s, 5B), −22.7 (s, 1B) ppm.

¹³C {1H} NMR (125.7 MHZ, CDCl₃): δ=168.4 (s, 1C), 130.2 (s, 1C), 126.9 (s, 1C), 68.9 (s, 1C), 51.5 (s, 1C), 28.9 (s, 1C), 24.9 (s, 1C) ppm.

IR (ATR): [symbol] =3379 (w, v(N—H)), 3079 (vw, v(=CH)), 2959 (s, v(C—H)), 2873 (m, v(C—H)), 2462 (s, v(B—H)), 1663 (m, v(C═O)), 1472 (m, v(C═C)) cm⁻¹.

### Elemental Analysis:

Calculated for [nBu₄N] [1-H₃C₂C(O)HN(CH₂)₄O-closo-B₁₂H₁]: C, 61.00; H, 12.47; N, 5.47%.

Found for C₃₉H₉₅B₁₂N₃O₂: C, 58.46; H, 12.00; N, 4.77%.

### 1.6 General Protocol for the Reaction of amino-functionalized adamantane and closo-boron Cluster Derivatives with Eshmuno®_{epoxy} Materials

### 1.6.1 Eshmuno®_{epoxy} Starting Material

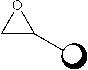

Elemental analysis: Found: C, 57.58; H, 8.50; N, 10.51%.

IR (ATR): [symbol] =3420 (m, v(O—H)), 2934 (s, v(C—H)), 2866 (m, v(C—H)), 1687 (m, v(C═O)), 1488 (m, v(C═C)) cm⁻¹.

A defined amount of sodium borohydride and sodium hydroxide is dissolved in 2-propanol under stirring followed by the addition of —OH functional groups baring stationary phase, Eshmuno® base beads. The obtained suspension is stirred for 30 minutes following by the addition of butan-diol-glycidyl-ether under inert conditions. The reaction mixture is stirred for 21 hours at room temperature following with an addition of acetic acid and further reaction time for 1 hour. The produced epoxy group containing stationary material is extensively washed with 2-propanol and water to remove the non-reacted components. Finally the obtained stationary phase (Eshmuno®_{epoxy}) can be dried and stored for later use.

The Eshmuno®_{epoxy} material (5 g, dry, density of epoxy functions on the surface: 554 μeq g⁻¹) was washed with water (2 L). The moist material was transferred into a 500 mL 3-neck flask, equipped with a KPG stirrer, and suspended in water (150 mL). The corresponding amino derivative (5.07 mmol) and lithium bromide (436 mg, 5.07 mmol) were dissolved in water (100 mL), and this solution was added at 60° C. to the suspension of Eshmuno®_{epoxy} in water. The resulting suspension was stirred for 4 hours at 120 rpm and filtered. The solid material was washed with water (1 L), warm water (60° C., 500 mL) and dried in a fine vacuum.

### 1.6.2 Eshmuno®_{Amino Adamantane}

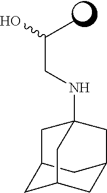

Quantity of amino-functionalized adamantane used for this synthesis: 766 mg 1-H₂N—C₁₀H₁₅ (5.07 mmol).

### Characterization:

Elemental analysis, found: C, 58.50; H, 8.74; N, 10.14%.

IR (ATR): [symbol] =3420 (m, v(O—H)), 2934 (s, v(C—H)), 2866 (m, v(C—H)), 2860 (m, v(C—H)), 1687 (m, v(C—O)), 1488 (m, v(C═C)) cm⁻¹.

### 1.6.3 Eshmuno®_{HN-doso-CB11H11}

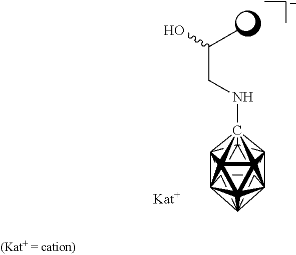

Quantity of K[1-H₂N-closo-CB₁₁H₁₁] used for this synthesis: 1.00 g (5.07 mmol).

### Characterization:

Elemental analysis, found: C, 54.66; H, 8.36; N, 10.21; B, 2.38%.

¹¹B [¹H} MAS-NMR (128.4 MHZ, vᵣₒₜ=15 KHZ): δ=−13.8 (s, 11B) ppm. IR (ATR): [symbol] =3420 (m, v(O—H)), 2934 (s, v(C—H)), 2866 (m, v(C—H)), 2553 (m, v(B—H)), 1687 (m, v(C—O)), 1488 (m, v(C═C)) cm⁻¹.

### 1.6.4 Eshmuno®_{HN-12-I-closo-CB11H10}

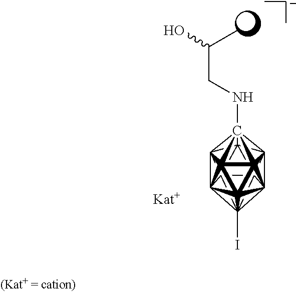

Quantity of Cs[1-H₂N-12-I-closo-CB₁₁H₁₀] used for this synthesis: 2.11 g (5.07 mmol).

### Characterization:

Elemental analysis, found: C, 54.37; H, 8.16; N, 11.35; B, 1.91%.

¹¹B{¹H} MAS-NMR (128.4 MHZ, vᵣₒₜ=15 kHz): δ=−12.4 (s, 11B), −19.4 (s, 1B) ppm.

IR (ATR): [symbol] =3420 (m, v(O—H)), 2934 (s, v(C—H)), 2866 (m, v(C—H)), 2565 (m, v(B—H)), 1687 (m, v(C—O)), 1488 (m, v(C═C)) cm⁻¹.

### 1.7 General Procedure for the Functionalization of the Eshmuno® Materials

### 1.7.1 Eshmuno®_{hydroxy} starting material

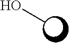

Elemental analysis, Found: C, 53.47; H, 7.87; N, 10.98%.

IR (ATR): [symbol] =3419 (m, (O—H), 2935 (s, v(C—H), 2865 (m, v(C—H), 1687 (m, v(C═O)), 1488 (m, v(C═C)) cm⁻¹.

The syntheses were carried under an inert gas atmosphere similar to the synthesis described in the literature (Graalfs, H.; Graft Copolymer for Cation-exchange Chromatography. Merck Patent GmbH, WO2008145270, 2008.). The starting Eshmuno® material, which was stored under an 20% ethanolic aqueous solution, was filtered and washed with distilled water (2 L). Afterwards, the Eshmuno®_{hydroxy} material was stored under water (50 mL) for one day. After 24 hours, the Eshmuno®_{hydroxy} material was filtered and transferred into a 500 mL 3-neck flask, equipped with a KPG stirrer, a drip funnel, a reflux condenser, and an inlet tube for nitrogen gas. The acrylic acid or other allylic derivatives (5.00 mmol) were dissolved in a solvent (180 mL) and added to the Eshmuno®_{hydroxy} material. The initiator, consisting of ammonium cerium (IV) nitrate (850 mg, 1.55 mmol), concentrated nitric acid (631 mg, 0.417 mL, 10.0 mmol), and water (70 ml), was added as soon as possible to the reaction mixture under vigorous stirring. Subsequently, the suspension was stirred for four hours at 30° C. and 120 rpm. The functionalized Eshmuno® material was filtered, washed with water (3×250 mL), 1 M sulfuric acid and 0.2 M ascorbic acid (8×250 ml), water (3×250 mL), warm water (60° C.; 10×250 ml), water (2×250 mL), 1 M sodium hydroxide solution (2×250 mL), water (2×250 ml), 70% ethanolic aqueous solution (2×250 ml), water (2×250 ml), 20% ethanolic aqueous solution, containing 150 mM sodium chloride (2×250 ml) and stored in a 20% ethanolic aqueous solution containing 150 mM sodium chloride.

### 1.7.2 Syntheses of Eshmuno®_{amidocarboxy-adamantyl}

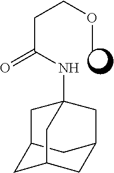 [SMILES: COCCC(=O)NC12CC3CC(CC(C3)C1)C2]

Quantities of Chemicals used for this Synthesis:
1.03 g (5.00 mmol) of 1-H₃C₂C(O)HN—C₁₀H₁₅;
40 mL Eshmuno®_{hydroxy} suspension.
Solvent: 105 mL of water and 75 mL tert-butanol.

### Product Characterization:

Elemental analysis, found: C, 62.22; H, 8.49; N, 10.37%.

IR (ATR): [symbol] =3419 (m, v(O—H)), 2935 (s, v(C—H)), 2910 (m, v(C—H)), 2865 (m, v(C—H)), 1687 (m, v(C—O)), 1488 (m, v(C═C)) cm⁻¹.

### 1.7.3 Syntheses of Eshmuno®_{1-amidocarboxy-closo-1,2-C2B10H11}

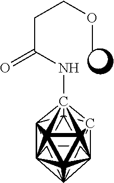

Quantities of Chemicals used for this Synthesis:
1.13 g (5.00 mmol) of the 1-H₃C₂C(O)HN-closo-1,2-C₂B₁₀H₁₁;
40 mL Eshmuno®_{hydroxy} suspension.
Solvent: 105 mL of water and 75 mL tert-butanol.

### Product Characterization:

Elemental analysis, found: C, 54.06; H, 8.64; N, 11.66; B, 1.47%.

¹¹B{¹H} MAS-NMR (128.4 MHz, vᵣₒₜ=15 kHz): δ=−13.7 (s, 10B) ppm.

IR (ATR): [symbol] =3419 (m, v(O—H)), 2935 (s, v(C—H)), 2865 (m, v(C—H)), 2591 (m, v(B—H)), 1687 (m, v(C═O)), 1488 (m, v(C═C)) cm⁻¹.

### 1.7.4 Syntheses of Eshmuno®_{1-amidocarboxy-closo-CB11H11}

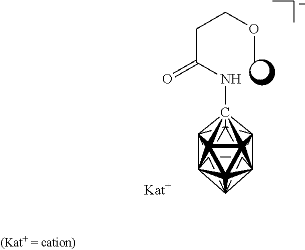

Quantities of chemicals used for this Synthesis:
1.13 g (5.00 mmol) of the K[1-H₃C₂ (O) CHN-closo-CB₁₁H₁₁];
40 mL Eshmuno®_{hydroxy} suspension.
Solvent: 180 ml of water.

### Product Characterization:

Elemental analysis, found: C, 50.88; H, 7.95; N, 10.24; B, 1.09%.

¹¹B{₁} MAS-NMR (128.4 MHZ, vᵣₒₜ=15 kHz): δ=−14.0 (s, 11B) ppm.

IR (ATR): [symbol] =3419 (m, v(O—H)), 2935 (s, v(C—H)), 2865 (m, v(C—H)), 2547 (m, v(B—H)), 1687 (m, v(C—O)), 1488 (m, v(C═C)) cm⁻¹.

### 1.8 Preparation of Functionalized Eshmuno® Materials by the Reaction of adamantane and closo-boron Cluster Derivatives Containing amino-group with Eshmuno®_{COO} Materials

### 1.8.1 Syntheses of Eshmuno®_{carboxy} Starting Materials

The synthesis of the COO-containing starting material, necessary about of the acrylic acid is dissolved in water and the pH is adjusted, if necessary to pH 2.2. The mixture is stirred at a temperature between 0-5° C. to obtain a homogeneous solution. Afterwards the —OH containing base material is added, e.g. Eshmuno® particles. Polymerization is started by adding Cerium (IV) nitrate. The reaction takes place for 4 hours at 30-50° C. After the polymerization reaction the non-reacted components are removed by extensive washing using acidic, basic and solvent mixtures at room or elevated temperatures.

### Eshmuno®_{COO-200}

Quantities of Chemicals used for this Synthesis:
1.19 g (16.5 mmol) of acrylic acid;
250 ml of an Eshmuno® suspension.

### Product Characterization:

Elemental analysis, found: C, 55.89; H, 8.51; N, 11.82%.

IR (ATR): [symbol] =3419 (m, v(O—H)), 2935 (s, (C—H)), 2865 (m, v(C—H)), 1687 (m, v(C═O)), 1488 (m, v(C═C)) cm⁻¹.
Carboxylic group density: 198 μeq g⁻¹

### Eshmuno®_{COO-700}

Quantities of Chemicals used for this sSynthesis:
3.96 g (55.0 mmol) of acrylic acid;
250 mL of an Eshmuno® suspension.

### Product Characterization:

Elemental analysis, found: C, 56.98; H, 8.44; N, 11.73%.

IR (ATR): [symbol] =3419 (m, v(O—H)), 2935 (s, v(C—H)), 2865 (m, v(C—H)), 1687 (m, v(C═O)), 1488 (m, v(C═C)) cm⁻¹.

Carboxylic group density: 679 μeq g-1

### Eshmuno®_{COO-1000}

Quantities of chemicals used for this synthesis:
5.94 g (82.5 mmol) of acrylic acid;
250 mL of an Eshmuno® suspension.

### Product Characterization:

Elemental analysis, found: C, 56.03; H, 8.25; N, 10.89%.

IR (ATR): [symbol] =3419 (m, v(O—H)), 2935 (s, v(C—H)), 2865 (m, v(C—H)), 1687 (m, v(C═O)), 1488 (m, v(C═C)) cm⁻¹.
Carboxylic group density: 1053 μeq g-1

### Eshmuno®_{COO-2400}

Quantities of Chemicals used for this Synthesis:
15.9 g (220.0 mmol) of acrylic acid;
250 mL of an Eshmuno® suspension.

### Product Characterization:

Elemental analysis, found: C, 55.89; H, 8.51; N, 11.82%.

IR (ATR): [symbol] =3419 (m, v(O—H)), 2935 (s, v(C—H)), 2865 (m, v(C—H)), 1687 (m, v(C═O)), 1488 (m, v(C═C)) cm⁻¹.
Carboxylic group density: 2366 μeq g⁻¹

The syntheses presented below were carried out under an inert gas atmosphere similar to syntheses described in the literature (Graalfs, H.; Graft Copolymer for Cation-exchange Chromatography. Merck Patent GmbH, WO2008145270, 2008.).

### General Protocol:

The Eshmuno®_{COO} material (300 μeq g-1) stored in 20% ethanolic aqueous solution containing sodium chloride (150 mM), was filtered and washed with water (2 L). Afterward, the Eshmuno®_{COO} material (5 mL gel height) was stored in water (25 mL). After 24 hours, the Eshmuno®_{COO} material was filtered and transferred into a 250 mL round flask. The amine derivative of adamantane or closo-boron cluster was dissolved in water (100 mL) and added to the Eshmuno®_{COO} material. The suspension was shaken for 17 hours, heated to 60° C. and 1-ethyl-3-(3-dimethylamino-propyl) carbodiimide (EDC) was added. After 3 hours, EDC was added again. The Eshmuno® functionalized material has been filtered, washed with water (4×50 mL), 1 M sodium chloride solution (3×50 mL), 0.1 M sodium phosphate buffer (pH=7) (4×50 mL), 20% ethanolic aqueous solution containing sodium chloride (150 mM) (2×50 mL)) and stored in a 20% ethanolic aqueous solution containing sodium chloride (150 mM).

Syntheses using 5 mL-Eshmuno®_{COO}-Gel starting material:

### 1.8.2 Syntheses of Eshmuno®_{amidocarboxy-adamantyl}

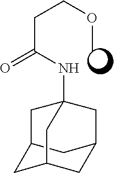 [SMILES: COCCC(=O)NC12CC3CC(CC(C3)C1)C2]

Quantities of Chemicals used for this Synthesis:
766 mg (5.00 mmol) of 1-H₂N—C₁₀H₁₅;
1.00 g+1.00 g EDC (5.22 mmol+5.22 mmol).
Eshmuno®_{COO}

### Product Characterization:

Elemental analysis, found: C, 55.84; H, 8.78; N, 13.59%.

IR (ATR): [symbol] =3419 (m, v(O—H)), 2935 (s, v(C—H)), 2865 (m, v(C—H)), 2860 (w, v(C—H)), 1687 (m, v(C═O)), 1488 (m, v(C═C)) cm⁻¹.

### 1.8.3 Syntheses of Eshmuno®_{1-amidocarboxy-dloso-CB11H11}

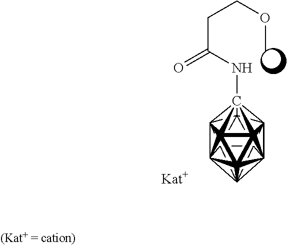

Quantities of Chemicals used for this Synthesis:
1.00 g (5.00 mmol) of the K[1-H₂N-closo-CB₁₁H₁₁];
1.00 g+1.00 g EDC (5.22 mmol+5.22 mmol).
Eshmuno®_{COO}

### Product Characterization:

Elemental analysis, found: C, 52.76; H, 7.87; N, 11.48; B, 1.34%.

¹¹B{¹H} MAS-NMR (128.4 MHZ, vᵣₒₜ=14.5 kHz): δ=−13.6 (s, 11B) ppm.

IR (ATR): [symbol] =3419 (m, v(O—H), 2935 (s, v(C—H), 2865 (m, v(C—H), 2538 (m, v(B—H)), 1687 (m, v(C—O)), 1488 (m, v(C═C)) cm⁻¹.

### 1.8.4 Syntheses of Eshmuno®_{amidocarboxy-doso-B10H9}

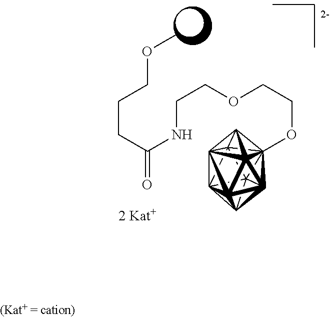

Quantities of Chemicals used for this Synthesis:
2.38 g (5.00 mmol) Cs₂[2-H₂N(CH₂)₂O(CH₂)₂O-closo-B₁₀H₉];
1.00 g+1.00 g EDC (5.22 mmol+5.22 mmol).
Eshmuno®_{COO}

### Product Characterization:

Elemental analysis, found: C, 53.16; H, 8.54; N, 13.32;

B, 0.57%.

¹¹B{¹H} MAS-NMR (128.4 MHZ, vᵣₒₜ=14 kHz): δ=6.6 (s, 1B), −1.8 (s, 2B), −25.1 (s, 4B), −31.4 (s, 3B) ppm.

IR (ATR): [symbol] =3419 (m, v(O—H)), 2935 (s, v(C—H)), 2865 (m, v(C—H)), 2453 (m, v(B—H)), 1687 (m, v(C═O)), 1488 (m, v(C═C)) cm⁻¹

### 1.8.5 Syntheses of Eshmuno®_{amidocarboxy-doso-B12H11}

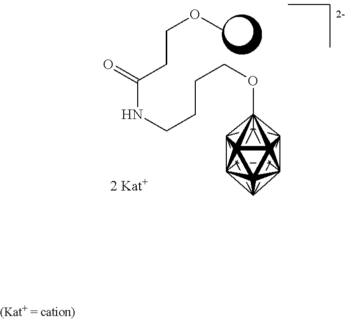

Quantities of Chemicals used for this Synthesis:
3.22 g (6.51 mmol) of the Cs₂[1-H₂N(CH₂)₄O-closo-B₁₂H₁₁];
1.30 g+1.30 g EDC (6.78 mmol+6.78 mmol),
Eshmuno®_{COO-300}.

### Product Characterization:

Elemental analysis, found: C, 53.51; H, 8.63; N, 11.54; B, 1.66%.

¹¹B {1H} MAS-NMR (128.4 MHZ, vᵣₒₜ=14 kHz): δ=6.3 (s, 1B), −17.0 (s, 10 B), −22.8 (s, 1B) ppm.

IR (ATR): [symbol] =3419 (m, v(O—H)), 2935 (s, v(C—H)), 2865 (m, v (C—H)), 2576 (m, v(B—H)), 1687 (m, v(C═O)), 1488 (m, v(C═C)) cm⁻¹.

### 1.8.6 Syntheses of Eshmuno®_{oxo-amidocarboxy-doso-B12H11}

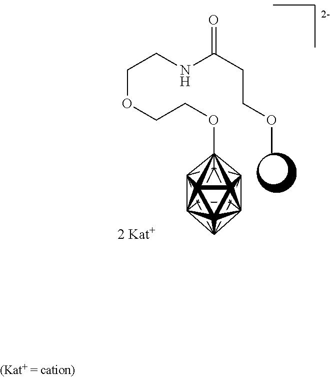

Quantities of Chemicals used for this Synthesis:
2.50 g (5.00 mmol) Cs₂[1-H₂N(CH₂)₂O(CH₂)₂O-closo-B₁₂H₁₁];
1.00 g+1.00 g EDC (5.22 mmol+5.22 mmol).
Eshmuno®_{COO}

### Product Characterization:

Elemental analysis, found: C, 54.49; H, 8.54; N, 12.04; B, 0.64%.

¹¹B{¹H} MAS-NMR (128.4 MHZ, vᵣₒₜ=14 kHz): δ=6.3 (s, 1B), −17.0 (s, 10 B), −22.8 (s, 1B) ppm.

IR (ATR): [symbol] =3419 (m, v(O—H)), 2935 (s, v(C—H)), 2865 (m, v(C—H)), 2576 (m, v(B—H)), 1687 (m, v(C═O)), 1488 (m, v(C═C)) cm⁻¹.

Syntheses using 50 mL-Eshmuno@coo-Gel starting material:

### General Protocol

The Eshmuno®_{COO} material, which was stored under 20% ethanolic aqueous solution containing sodium chloride (150 mM), was filtered and washed with water (2 L). Afterwards, the Eshmuno®_{COO} material (50 mL gel height) was stored for 24 hours in water (250 mL). Then, the Eshmuno®_{COO} material was washed with additional water (3×500 mL), filtered, transferred into a 1 L round-bottom flask, and suspended in water (100 mL). The amino derivate of adamantane or a closo-boron cluster and 1-ethyl-3-(3-dimethylaminopropyl) carbodiimide (EDC) were added to this suspension. The reaction mixture was stirred (115 rpm) at 60° C. for 3 hours. After three hours, additional EDC was added and the reaction mixture was stirred for further 17 hours at 60° C. and 115 rpm. The Eshmuno@-functionalized material was filtered, washed with water (4×50 mL), 1 M sodium chloride solution (3×50 mL), 0.1 M sodium phosphate buffer (pH=7) (4×50 mL), 20% ethanolic aqueous solution containing sodium chloride (150 mM) (2×50 mL), and stored in a 20% ethanolic aqueous solution containing sodium chloride (150 mM).

### 1.8.7 Syntheses of Eshmuno®_{oxo-amidocarboxy-closo-B12H11}

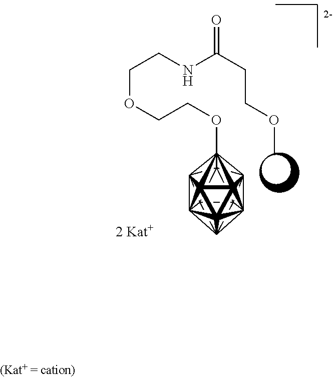

Quantities of Chemicals used for this Synthesis:
1.25 g (2.41 mmol) of Cs₂[1-H₂N(CH₂)₂O(CH₂)₂O-closo-B₁₂H₁₁];
0.60 g+0.60 g (1.57 mmol+1.57 mmol) EDC;
50 mL Eshmuno®_{COO-200}.

### Product Characterization:

Elemental analysis, found: C, 53.80; H, 8.64; N, 11.59; B, 0.50%.

¹¹B{¹H} MAS-NMR (128.4 MHZ, vᵣₒₜ=14.6 kHz): δ=6.3 (s, 1B), −17.0 (s, 10B), −22.8 (s, 1B) ppm.

IR (ATR): [symbol] =3419 (m, v(O—H)), 2935 (s, v (C—H)), 2865 (m, w (C—H)), 2480 (vw, v(B—H)), 1687 (m, v(C═O)), 1488 (m, v(C═C)) cm⁻¹.

Residual carboxylic group density: 54 μeq g⁻¹.

### 1.8.8 Syntheses of Eshmuno®_{oxo-amidocarboxy-closo-B12H11}

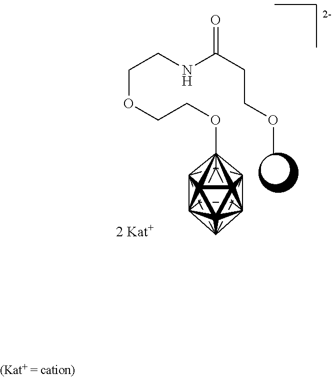

Quantities of Chemicals used for this Synthesis:
3.32 g (6.51 mmol) of the CS₂[1-H₂N(CH₂)₂O(CH₂)₂O-closo-B₁₂H₁₁];
2.24 g+2.24 g (5.90 mmol+5.90 mmol) EDC;
50 mL Eshmuno®_{COO-700}.

### Product Characterization:

Elemental analysis, found: C, 52.99; H, 8.57; N, 11.24; B, 1.33%.

¹¹B{¹H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3419 (m, v(O—H)), 2935 (s, v(C—H)), 2865 (m, v(C—H)), 2480 (w, v(B—H), 1687 (m, v(C═O)), 1488 (m, v(C═C)) cm⁻¹.

Residual carboxylic group density: 158 μeq g⁻¹.

### 1.8.9 Syntheses of Eshmuno®_{oxo-amidocarboxy-closo-B12H11}

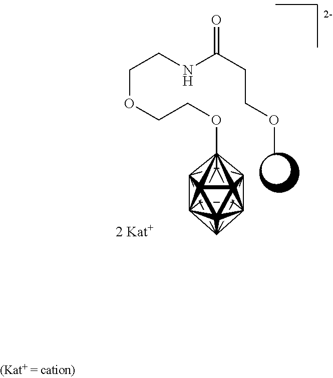

Quantities of Chemicals used for this Synthesis:
6.56 g (12.9 mmol) of the Cs₂[1-H₂N(CH₂)₂O(CH₂)₂O-closo-B₁₂H₁₁];
4.50 g+4.50 g (11.8 mmol+11.8 mmol) EDC;
50 mL Eshmuno®_{COO-1000}.

### Product Characterization:

Elemental analysis, found: C, 50.28; H, 8.50; N, 10.71; B, 2.13%.

¹¹B{¹H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3419 (m, O—H)), 2935 (s, v(C—H)), 2865 (m, v(C—H)), 2480 (w, v(B—H)), 1687 (m, v(C—O)), 1488 (m, v(C═C)) cm⁻¹.

Residual carboxylic group density: 379 μeq g⁻¹.

### 1.8.10 Syntheses of Eshmuno®_{oxo-amidocarboxy-doso-B12H11}

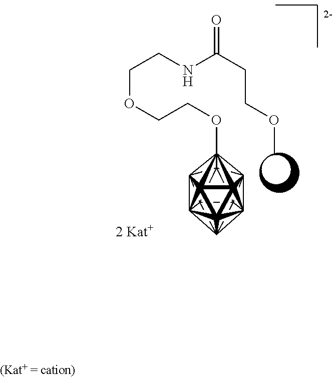

Quantities of Chemicals used for this Synthesis:
11.5 g (22.5 mmol) of Cs₂[1-H₂N(CH₂)₂O(CH₂)₂O-closo-B₁₂H₁₁];
7.90 g+7.90 g (20.6 mmol+20.6 mmol) EDC;
50 mL Eshmuno®_{COO-2400}.

### Product Characterization:

Elemental analysis, found: C, 49.20; H, 8.53; N, 10.75; B, 2.22%.

¹¹B{¹H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3419 (m, v(O—H)), 2935 (s, v(C—H)), 2865 (m, v(C—H)), 2480 (w, v(B—H)), 1687 (m, v(C—O)), 1488 (m, v(C═C)) cm⁻¹.

Residual carboxylic group density: 940 μeq g⁻¹.

### 1.8.11 Syntheses of Eshmuno®_{oxo-amidocarboxy-COSAN}

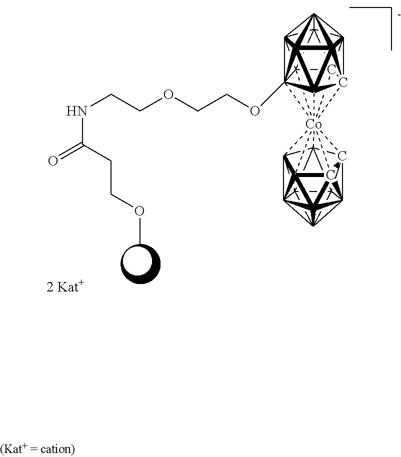

The synthesis was performed by a slight modification of the aforementioned general method:

Eschmuno®_{COO} (6.0 mL, 1000 μeq g⁻¹) was washed with water (1 L) and stored in water (40 mL) for 24 hours. The particles were washed with water (2×500 mL) and transferred into a 250 mL Schlenk flask. [3,3′-Co(8-{NH₃CH₂CH₂OCH₂CH₂O}-1,2-C₂B₉H₁₀) (1′,2′-C₂B₉H₁₁)] (689 mg, 1.61 mmol) was treated with KOH (90 mg, 1.61 mmol) in a mixture of water (50 mL) and tert-butanol (50 mL). The resulting solution was added to the BDM particles under an inert gas atmosphere. EDC (1-ethyl-3-(3-dimethylaminopropyl) carbodiimide) (253 mg, 1.32 mmol) was added and the mixture was shaken at a rotary evaporator (120 rpm) at 60° C. After 3 hours, additional EDC (253 mg, 1.32 mmol) was added and the reaction mixture was shaken at a rotary evaporator (120 rpm) for further 21 hours. The Eschmuno®_{COO} material was filtered off. The residue was rinsed with water (2×50 mL) and with aqueous sodium chloride (2×250 mL, 1 M), sodium phosphate buffer solution (3×250 mL, pH=7, 50 mM), and an aqueous solution of ethanol (3×250 mL, 20% v/v) containing sodium chloride (150 mM). The moist material was stored in an aqueous solution of ethanol (20% v/v) containing sodium chloride (150 mM).

### Product Characterization:

IR (ATR): [symbol] =3391 (vs vbr, v(O—H)), 2935 (s, v(C—H)), 2865 (m, v(C—H)), 2557 (s, v(B—H)), 1672 (vs, v(C═O)) cm⁻¹.

### 1.8.12 Syntheses of Eshmuno®_{oxo-amidocarboxy-nido-C2B9H11}

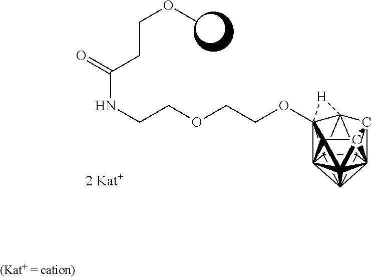

The synthesis was performed by a slight modification of the aforementioned general method:

Eschmuno®_{COO} (9.5 mL, 1000 μeq g-1) was washed with water (1 L) and stored in water (40 mL) for 24 hours. The particles were washed with water (2×500 mL) and transferred into a 250 mL Schlenk flask. 10-(NH₃CH₂CH₂OCH₂CH₂O)-nido-7,8-C₂B₉H₁₁ (596 mg, 2.51 mmol) was treated with KOH (140 mg, 2.51 mmol) in water (100 mL). The resulting solution was added to BDM particles under an inert gas atmosphere. EDC (1-ethyl-3-(3-dimethylaminopropyl) carbodiimide) (402.0 mg, 2.09 mmol) was added and the mixture was shaken at a rotary evaporator (120 rpm) at 60° C. After 3 hours, additional EDC (402.0 mg, 2.09 mmol) was added and the reaction mixture was shaken for further 21 hours at 60° C. The Eschmuno®_{COO} material was filtered off. The residue was rinsed with water (2×50 mL) and washed with aqueous sodium chloride (2×250 mL, 1 M), sodium phosphate buffer solution (3×250 mL, pH=7, 50 mM), and an aqueous solution of ethanol (3×250 mL, 20% v/v) containing sodium chloride (150 mM). The moist material was stored in an aqueous solution of ethanol (20% v/v) containing sodium chloride (150 mM).

### Product Characterization:

IR (ATR): [symbol] =3395 (vs vbr, v(O—H)), 2935 (s, v(C—H)), 2869 (m, v(C—H)), 2527 (m, v(B—H)), 1675 (vs, v(C═O)) cm⁻¹.

### 1.8.13 Syntheses of Eshmuno®_{oxo-amidocarboxy-cioso-B12H11}

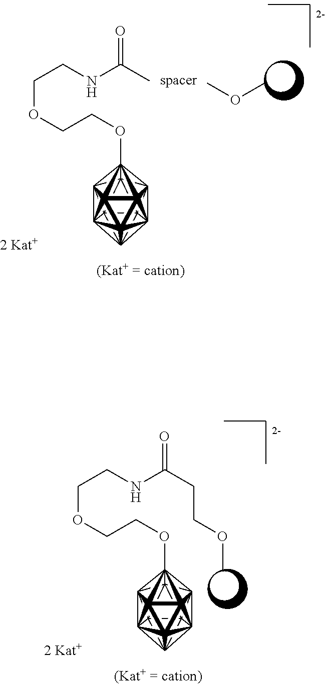

Eschmuno®_{COO} (11.0 mL, 1000 μeq g-1) was washed with water (1 L) and stored in water (40 mL) for 20 hours. The particles were washed with water (2×500 mL) and transferred into a 250 mL Schlenk flask. Cs₂[1-H₂N(CH₂)₂O(CH₂)₂O-closo-B₁₂H₁₁] (1.35 g, 2.64 mmol) was added. A mixture of water (50 mL) and tert-butanol (50 mL) was added to the BDM particles under an inert gas atmosphere. EDC (1-ethyl-3-(3-dimethyl-aminopropyl) carbodiimide) (464 mg, 2.42 mmol) was added and the mixture was shaken at a rotary evaporator (120 rpm) at 60° C. After 3 hours, additional EDC (464 mg, 2.42 mmol) was added and the reaction mixture was shaken at a rotary evaporator (120 rpm) for further 21 hours. The Eschmuno@coo material was filtered off. The residue was rinsed with water (2×500 mL) and with aqueous sodium chloride (2×250 mL, 1 M), sodium phosphate buffer solution (3×250 mL, pH=7, 50 mM), and an aqueous solution of ethanol (3×250 mL, 20% v/v) containing sodium chloride (150 mM). The moist material was stored in an aqueous solution of ethanol (20% v/v) containing sodium chloride (150 mM).

### Product Characterization:

IR (ATR): [symbol] =3392 (vs vbr, v(O—H)), 2940 (s, v(C—H)), 2861 (m, v(C—H)), 2478 (m, v(B—H)), 1688 (vs, v(C═O)) cm⁻¹.

### 1.9 General Protocol for the Synthesis of Chromatography Materials by Modification of Macro-Prep CM Resin, Toyopearl® HW-65F and Sepharose® 4B with Closo-Dodecaborate Anions

Gravity sedimented starting material (5.00 mL) was washed with water (500 mL) and stored in water (25 mL) for 24 hours. The material was washed again with water (250 mL). The moist gel was placed into a 100 mL round bottom flask together with Cs₂[1-H₂N(CH₂)₂O(CH₂)₂O-closo-B₁₂H₁₁] and water (50 mL). The reaction mixture was heated to 60° C. and 1-ethyl-3-(3-dimethylaminopropyl) carbodiimide (EDC) was added while shaking. After 3 hours, an additional amount of EDC was added and the suspension was shaken for 17 hours at 60° C. The material was filtered, washed with water (4×50 mL), 1 M sodium chloride solution (3×50 mL), 0.1 M sodium phosphate buffer (pH=7) (4×50 mL), 20% ethanolic aqueous solution containing sodium chloride (150 mM) (2×50 mL), and stored in an 20% ethanolic aqueous solution containing sodium chloride (150 mM).

### Characterization of the Starting Materials:

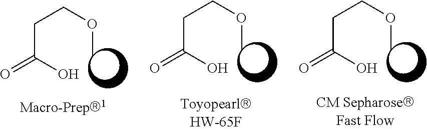

Elemental analysis, found: C, 50.43; H, 7.62%.

IR (ATR): [symbol] =3410 (m, v(O—H)), 2939 (s, v(C—H)), 1721 (m, (C═O)), 1450 (m, v (C═C)) cm⁻¹ Carboxylic groups density: 210 μeq g⁻¹.

Elemental analysis, found: C, 50.45; H, 7.71; N, 0.32%.

IR (ATR): [symbol] =3434 (m, v(O—H)), 2941 (s, v(C—H)), 1723 (m, v(C═O)), 1450 (m, v(C═C)) cm⁻¹

Carboxylic groups density: 550 μeq g⁻¹.

Elemental analysis, found: C, 42.04; H, 6.38%.

IR (ATR): [symbol] =3436 (m, v(O—H)), 2877 (s, v(C—H)), 1699 (m, v(C═O)), 1400 (m, v(C═C)) cm⁻¹

Carboxylic groups density: 50 mg mL⁻¹.

### 1.9.1 Macro-Prep®_{B12}

Quantities of Chemicals used for this Synthesis:
1.24 g (2.42 mmol) of the CS₂[1-H₂N(CH₂)₂O(CH₂)₂O-closo-B₁₂H₁₁]; 0.85 g+0.85 g (2.22 mmol+2.22 mmol) of EDC.

Macro-Prep CM Resin (Macro-Prep CM Support) is a commercially available weak cation exchange support and obtained from Bio-Rad.

### Product Characterization:

Elemental analysis, found: C, 53.06; H, 7.78; N, 1.22; B, 1.10%, 11B {′H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3410 (m, MO-H)), 2939 (s, w (C—H)), 2480 (m, v(B—H)), 1721 (m, v(C═O)), 1450 (m, v(C═C)) cm⁻¹.

Toyopearl® HW-65F_{B12} Quantities of chemicals used for this synthesis:

740 mg (1.44 mmol) of the Cs₂ [1-H₂N(CH₂)₂O(CH₂)₂O-closo-B₁₂H₁₁]; 0.55 g+0.55 g (1.32 mmol+1.32 mmol) of EDC.

Toyopearl® HW-65F is a commercially available hydroxylated methacrylate chromatography support and obtained from Sigma-Aldrich.

### Product Characterization:

Elemental analysis, found: C, 52.24; H, 7.80; N, 1.21; B, 0.51%. 11B {1H} MAS-NMR (128.4 MHZ, vᵣₒₜ=14.8 kHz): δ=6.3 (s, 1B), −17.0 (s, 10B), −22.8 (s, 1B) ppm.

IR (ATR): [symbol] =3434 (m, MO-H)), 2941 (s, v(C—H)), 2480 (m, v(B—H)), 1723 (m, v(C═O)), 1450 (m, v(C═C)) cm⁻¹.

### 1.9.2 CM Sepharose® Fast Flow_{B12}

Quantities of Chemicals used for this Synthesis:
720 mg (1.40 mmol) of the Cs₂[1-H₂N(CH₂)₂O(CH₂)₂O-closo-B₁₂H₁₁];
495 mg+495 g (1.29 mmol+1.29 mmol) of EDC.
CM Sepharose® Fast Flow is a commercially available 6% cross-linked agarose weak cation exchange support and obtained from Sigma-Aldrich.

### Product Characterization:

Elemental analysis, found: C, 44.46; H, 6.97; N, 1.79; B, 1.57%.

¹¹B{¹H} MAS-NMR (128.4 MHZ, vᵣₒₜ=14.8 kHz): δ=6.3 (s, 1B), −17.0 (s, 10B), −22.8 (s, 1B) ppm.

IR (ATR): [symbol] =3436 (m, MO-H)), 2877 (s, v(C—H)), 2480 (m, v(B—H)), 1699 (m, v(C═O)), 1400 (m, v(C═C)) cm⁻¹.

### 1.10 One-Pot Synthesis for Chromatography Materials Based on Eshmuno®_{COO} Modified with Boron Clusters

### General Protocol

closo-Dodecaborate as triethylammonium or tetrabutylammonium salt was placed into a 2 L three neck flask, equipped with reflux condenser, KPG stirrer, inlet tube for nitrogen gas, and Na[BF₄] and [nBu₄N] Br (in case of triethylammonium closo-dodecaborate was used as starting material) were added. The mixture was suspended in dioxane (250 mL), HCl (4 M in dioxane) was added, and the suspension was warmed to 120° C. and stirred (120 rpm-1) for 24 hours. After that, the reaction mixture was cooled to 50° C. and gaseous ammonia was passed through the solution for 15 min. The reaction mixture was warmed to 120° C. again and stirred for 12 hours. The excess ammonia was removed by passing nitrogen through the reaction mixture at 80° C. for 60 minutes. After addition of water (100 ml) and the Eshmuno®_{COO} material (1050 μeq g 1) (50 mL gel), the pH of the suspension was adjusted to 4.7. EDC was added and the suspension was stirred (100 rpm) at 70° C. for 17 hours. In-between, after 3 hours, an additional quantity of EDC was added. The functionalized Eshmuno® material was filtered, washed with water (4×50 ml), 1 M sodium chloride solution (3×50 ml), 0.1 M sodium phosphate buffer (pH=7) (4×50 ml), 20% ethanolic aqueous solution containing sodium chloride (150 mM) (2×50 mL), and stored in a 20% ethanolic aqueous solution containing sodium chloride (150 mM).

### 1.10.1 One-Pot Synthesis of Eshmuno®_{oxo-amidocarboxy-cioso-B12H11}

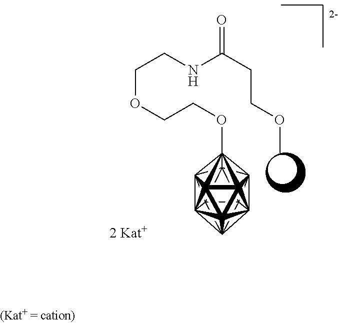

Quantities of chemicals used for this synthesis:
4.36 g (12.6 mmol) of [HNEt₃]₂[closo-B₁₂H₁₂];
8.93 g (27.7 mmol) of [nBu₄N] Br;
6.92 g (63.0 mmol) of Na[BF₄];
16 mL of HCl (4 M in dioxane);
5.63 g+5.63 g of EDC (14.7 mmol+14.7 mmol).

### Product Characterization:

Elemental analysis, found: C, 56.85; H, 8.32; N, 11.98; B, 0.52%. 11B {1H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3419 (m, MO-H)), 2935 (s, M (C—H)), 2865 (m, v (C—H)), 2481 (m, v(B—H)), 1687 (m, v(C═O)), 1488 (m, v(C═C)) cm⁻¹.

Residual carboxylic groups density: 87 μeq g⁻¹.

### 1.10.2 One-Pot Synthesis of Eshmuno®_{oxo-amidocarboxy-cioso-B12H11}

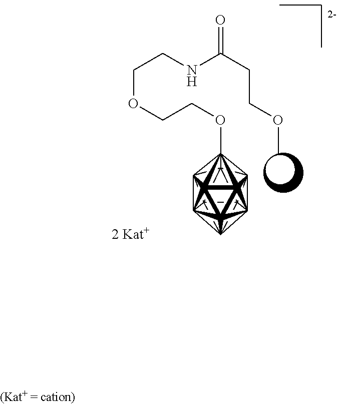

Quantities of chemicals used for this synthesis:
8.72 g (25.2 mmol) of [HNEt₃]₂[closo-B₁₂H₁₂];
17.9 g (55.5 mmol) of [nBu₄N] Br;
13.8 g (130.0 mmol) of Na[BF₄];
32 mL of HCl (4 M in dioxane);
5.63 g+5.63 g of EDC (14.7 mmol+14.7 mmol).

### Product Characterization:

Elemental analysis, found: C, 56.57; H, 8.37; N, 12.36; B, 0.82%.

¹¹B{¹H} MAS-NMR (128.4 MHz, vᵣₒₜ=14.8 KHz): δ=6.3 (s, 1B), −17.0 (s, 10B), −22.8 (s, 1B) ppm.

IR (ATR): [symbol] =3419 (m, v(O—H)), 2935 (s, v(C—H)), 2865 (m, v(C—H)), 2482 (m, v(B—H)), 1687 (m, v(C═O)), 1488 (m, v(C═C)) cm⁻¹.

Residual carboxylic groups density: 51 μeq g⁻¹.

### 1.10.3 One-Pot Synthesis of Eshmuno®_{oxo-amidocarboxy-cioso-B12H11}

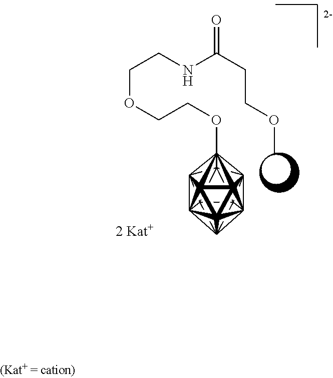

Quantities of Chemicals used for this Synthesis:
7.83 g (12.6 mmol) of [nBu₄N] 2 [closo-B₁₂H₁₂];
6.92 g (63.0 mmol) of Na[BF₄];
16 mL of HCl (4 M in dioxane);
5.63 g+5.63 g of EDC (14.7 mmol+14.7 mmol).

Instead of gaseous ammonia, an aqueous solution of NH₃ (25%) was used.

### Product Characterization:

Elemental analysis, found: C, 56.94; H, 8.23; N, 12.22; B, 0.63%.

¹¹B{¹H} MAS-NMR (128.4 MHZ, vᵣₒₜ=14.8 KHZ): δ=6.3 (s, 1B), −17.0 (s, 10B), −22.8 (s, 1B) ppm.

IR (ATR): [symbol] =3419 (m, v(O—H)), 2935 (s, v(C—H)), 2865 (m, v(C—H)), 2482 (m, v(B—H)), 1687 (m, v(C═O)), 1488 (m, v(C═C)) cm⁻¹.

Residual carboxylic groups density: 75 μeq g⁻¹.

### 1.10.4 One-Pot Synthesis of Eshmuno®_{oxo-amidocarboxy-cioso-B12H11}

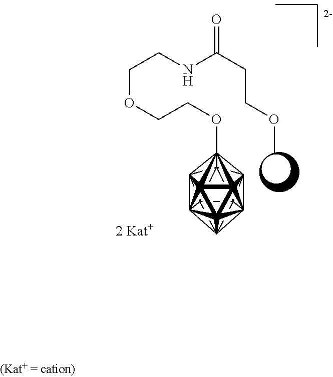

Quantities of Chemicals used for this Synthesis:
200 g (0.32 mol) of [nBu₄N]₂[closo-B₁₂H₁₂];
175 g (63.0 mmol) of Na[BF₄];
1.2 L of dioxane
239 mL of HCl (4 M in dioxane);
50.0 g+50.0 g of EDC (0.13 mol+0.13 mol).
500 mL gel (Eshmuno®_{COO-1050})

### Product Characterization:

Elemental analysis, found: C, 55.57; H, 8.47; N, 11.77; B, 1.21%.

¹¹B{¹H} MAS-NMR (128.4 MHZ, vᵣₒₜ=14.8 KHz): δ=6.3 (s, 1B), −17.0 (s, 10B), −22.8 (s, 1B) ppm.

IR (ATR): [symbol] =3419 (m, v(O—H)), 2935 (s, v(C—H)), 2865 (m, v(C—H)), 2482 (m, v(B—H)), 1687 (m, v(C═O)), 1488 (m, v(C═C)) cm⁻¹.

Residual carboxylic groups density: 140 μeq g⁻¹.

### 2. Application Examples

### 2.1 Use of Chromatography Material (as Described Above) for Separation of bovine serum albumin (BSA)

The boron cluster material as prepared according to Example 1.8.4, 1.8.5, and 1.8.7 to 1.8.10 (e.g. with the average particle size between 20-63 μm, the average pore size between 40-110 nm, COO-group density of the starting material of 200-2400 and a ligand density between 200-1460 μeq/g) was evaluated for its ability to bind and elute bovine serum albumin (BSA). The boron cluster modified material was packed into a chromatographic column of 5×50 mm dimensions with asymmetry between 0,8-1,2 and >3000 plates/m. After packing the boron cluster-modified material, the obtained chromatographic column was cleaned with 1 M NaOH solution for 30 minutes and preequilibrated by loading a buffer solution of pH of 6.0. The buffer solution contained sodium dihydrogen phosphate and NaOH or/and HCl to obtain the pH of 6.0. The same solution was used to dissolve the lyophilized BSA sample till 1 mg/ml concentration.

This solution was loaded onto the prepared chromatographic column till 10% break through values were reached. These steps and the following steps were performed at 75 cm/h buffer velocity. After loading BSA, the chromatographic column was washed with pH 6.0 buffer solution and then eluted used gradient elution with buffer having pH of 10.0 and 3M Imidazole. This elution buffer was prepared using different salts such as TRIS, Imidazole and NaCl. The conductivity and pH values were traced during the experimental set-up, showing that the BSA elution from the column was achieved due to the pH change during the gradient elution.

The sample elution was fractionated and obtained fractions were evaluated for the amount of the eluted BSA.

Eshmuno®COO without the boron cluster modification, as shown in example 1.8.1, does not bind BSA under the above mentioned conditions.

The analytical evaluation of collected fractions is displayed in Table 3, showing quantity of BSA bound per ml CV (column volume) of boron cluster modified material, ranging from ˜4 mg for 300 μeq COO-groups to 67 mg/ml for 1000 μeq modified material.

TABLE 3

| | | COO— group | | Bound Protein (mg/ml | | |
|---|---|---|---|---|---|---|
| | | density of the | Ligand | of column volume) | | |
| | Preparation | starting material | density | Run | Run | Run |
| Material | example | (μeq/g) | (μeq/g) | 1 | 2 | 3 |
| Eshmuno ®_{B12-200} | 1.8.7 | 200 | 146 | 0 | 0 | 0 |
| Eshmuno ®_{B10-300} | 1.8.4 | 300 | | 3.96 | 4.02 | 4.07 |
| Eshmuno ®_{B12-300} | 1.8.5 | 300 | | 6.20 | 6.17 | 6.12 |
| Eshmuno ®_{B12-2400} | 1.8.10 | 2400 | 1460 | 11.01 | 11.35 | 11.32 |
| Eshmuno ®_{B12-700} | 1.8.8 | 700 | 542 | 22.09 | 16.25 | 16.63 |
| Eshmuno ®_{B12-1000} | 1.8.9 | 1000 | 621 | 67.08 | 66.18 | 63.49 |

Increasing the group density/ligand density (starting material with COO-group density 2400 μeq/g) decreased the binding capacity of the BSA, suggesting that the optimum group density for the given modified starting material with COO-group density within 700-2400 μeq/g (ligand density between 542 to 1460 μeq/g). Moreover, the modified material was applied three times consequentially under the same conditions (run 1 to run 3) measuring the amount of the BSA in the elution fraction. The amounts of the eluted BSA were comparable within the runs enabling to implement this modified material numerous times.

### 2.2 Use of Chromatography Material (as Described Above) for Separation of trypsin in Varying pH and Conductivity Conditions

The boron cluster materials as prepared according to Examples 1.8.5 and 1.8.8 to 1.8.10 (e.g. with the average particle size between 20-63 μm, the average pore size between 40-110 nm, a group density between 400-900 μeq/g and a ligand density between 200-1460 μeq/g) was evaluated for its ability to bind trypsin in varying pH and conductivity conditions. The boron cluster-modified material was cleaned with 1 M NaOH solution for 30 minutes before subjecting to a vessel containing the corresponding buffer of certain pH and conductivity. The buffer solution contained sodium dihydrogen phosphate, sodium sulfate and NaOH or/and HCl. The pH of the buffer was varying between 4.5 to 7.5. The amount of Na₂SO₄: from 0 mM to 900 mM.

The same solution was used to dissolve the lyophilized trypsin sample till 5 mg/ml concentration. This solution was loaded onto the preconditioned chromatographic resin and incubated for 2 hours under constant stirring. After the solution containing the rest of not adsorbed protein was subtracted from the vessel. The trypsin concentration differences between the subjected and subtracted solution corresponded to the amount of the trypsin bound to the chromatographic material.

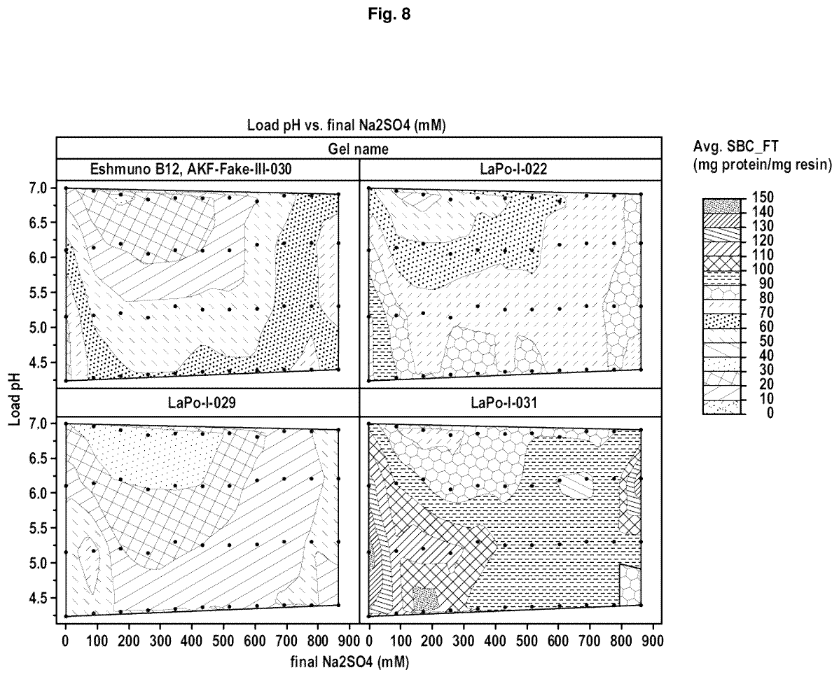

The analytical results of each selected pH and conductivity condition are displayed in the FIG. 8, showing the static binding capacity values for the trypsin binding on different boron cluster modified chromatographic materials (Eshmuno® B12-300 (bottom-left), Eshmuno® B12-700 (top-left), Eshmuno® B12-1000 (top-right), Eshmuno® B12-2400 (bottom-right)). FIG. 8 demonstrates strong binding at pH of 4.5-6.0 and low Na₂SO₄ concentrations and binding in elevated pH values of 6.0 and high Na₂SO₄ concentrations, thereby the conductivity of the solution is ˜90 mS/cm.

The amount of the bound protein was dependent on the ligand density: the higher the ligand density the higher the binding capacity, and the windows for the optimum binding remained for all selected boron cluster modified chromatographic materials.

### 2.3 Use of Chromatography Material Prepared in Aqueous and Organic Solvent (as Described Above) for Separation of Bovine Serum Albumin (BSA)

The boron cluster material as prepared according to Example 1.8.9 and 1.8.13 (e.g. with the average particle size between 20-63 μm, the average pore size between 40-110 nm, a group density between 400-900 μeq/g and a ligand density between 900-1000 μeq/g) was evaluated for its ability to bind and elute bovine serum albumin (BSA).

The boron cluster modified material was packed into a chromatographic column of 5×50 mm dimensions with asymmetry between 0,8-1,2 and >3000 plates/m. After packing the boron cluster-modified material, the obtained chromatographic column was cleaned with 1 M NaOH solution for 30 minutes and preequilibrated by loading a buffer solution of pH of 6.0. The buffer solution contained sodium dihydrogen phosphate and NaOH or/and HCl to obtain the pH of 6.0. The same solution was used to dissolve the lyophilized BSA sample till 1 mg/ml concentration. This solution was loaded onto the prepared chromatographic column till 10% break through values were reached. These steps and the following steps were performed at 75 cm/h buffer velocity. After loading BSA, the chromatographic column was washed with pH 6.0 buffer solution and then eluted used gradient elution with buffer having pH of 10.0 and 3M Imidazole. This elution buffer was prepared using different salts such as TRIS, Imidazole and NaCl. The conductivity and pH values were traced during the experimental set-up, showing that the BSA elution from the column was achieved due to the pH change during the gradient elution.

The sample elution was fractionated and obtained fractions were evaluated for the amount of the eluted BSA.

The analytical evaluation of collected fractions is displayed in the Table 4, showing the amount of BSA bound per ml CV of boron cluster modified material prepared in aqueous and organic solvents.

TABLE 4

| | | Amount of bound protein (mg/ml) | | |
|---|---|---|---|---|
| Material | Prepared as | Run 1 | Run 2 | Run 3 |
| TaKn-IV-301 | 1.8.9 | 35.12 | 34.95 | 34.98 |
| TaKn-IV-315 | 1.8.13 | 45.94 | 45.27 | 45.03 |

Both boron cluster modified materials showed binding capacity for BSA ranging from 34-46 mg/ml. Moreover, this range was maintained in the consequential implementation of the material for at least 3 runs.

### 2.4 Use of Chromatography Material (Nido-Cluster as Described Above) for Separation of Bovine Serum Albumin (BSA)

The boron cluster material as prepared according to Example 1.8.12 (e.g. with the average particle size between 20-63 μm, the average pore size between 40-110 nm and a group density between 400-900 μeq/g) was evaluated for its ability to bind and elute bovine serum albumin (BSA).

The boron cluster modified material was packed into a chromatographic column of 5×50 mm dimensions with asymmetry between 0,8-1,2 and >3000 plates/m. After packing the boron cluster-modified material, the obtained chromatographic column was cleaned with 1 M NaOH solution for 30 minutes and preequilibrated by loading a buffer solution of pH of 6.0. The buffer solution contained sodium dihydrogen phosphate and NaOH or/and HCl to obtain the pH of 6.0. The same solution was used to dissolve the lyophilized BSA sample till 1 mg/ml concentration.

This solution was loaded onto the prepared chromatographic column till 10% break through values were reached. These steps and the following steps were performed at 75 cm/h buffer velocity. After loading BSA, the chromatographic column was washed with pH 6.0 buffer solution and then eluted used gradient elution with buffer having pH of 10.0 and 3M Imidazole. This elution buffer was prepared using different salts such as TRIS, Imidazole and NaCl. The conductivity and pH values were traced during the experimental set-up, showing that the BSA elution from the column was achieved due to the pH change during the gradient elution.

The sample elution was fractionated and obtained fractions were evaluated for the amount of the eluted BSA.

The analytical evaluation of collected fractions is displayed in the Table 5, showing the amount of BSA bound per ml CV of boron nido-cluster modified material.

TABLE 5

| | | COO— | | | |
|---|---|---|---|---|---|
| | | group | | | |
| | | density of | | | |
| | | the starting | | | |
| | Prepared | material | Amount of bound protein (mg/ml) | | |
| Material | as | (μeq/g) | Run 1 | Run 2 | Run 3 |
| TaKn-IV- | 1.8.12 | 1000 | 23.28 | 23.94 | 24.56 |
| 305 | | | | | |
| TaKn-IV- | 1.8.12 | 450 | 10.64 | 17.55 | 16.22 |
| 311 | | | | | |

Both boron cluster modified materials showed binding capacity for BSA ranging from 10-25 mg/ml. Moreover, this range was maintained in the consequential implementation of the material for at least 3 runs.

### 2.5 Use of Chromatography Material (COSAN-Cluster as Described Above) for Separation of Bovine Serum Albumin (BSA)

The boron cluster material as prepared according to Example 1.8.11 (e.g. with the average particle size between 20-63 μm, the average pore size between 40-110 nm and a group ligand density of 1000 μeq/g) was evaluated for its ability to bind and elute bovine serum albumin (BSA).

The boron cluster modified material was packed into a chromatographic column of 5×50 mm dimensions with asymmetry between 0,8-1,2 and >3000 plates/m. After packing the boron cluster-modified material, the obtained chromatographic column was cleaned with 1 M NaOH solution for 30 minutes and preequilibrated by loading a buffer solution of pH of 6.0. The buffer solution contained sodium dihydrogen phosphate and NaOH or/and HCl to obtain the pH of 6.0. The same solution was used to dissolve the lyophilized BSA sample till 1 mg/ml concentration.

This solution was loaded onto the prepared chromatographic column till 10% break through values were reached. These steps and the following steps were performed at 75 cm/h buffer velocity. After loading BSA, the chromatographic column was washed with pH 6.0 buffer solution and then eluted used gradient elution with buffer having pH of 10.0 and 3M imidazole. This elution buffer was prepared using different salts such as TRIS, Imidazole and NaCl. The conductivity and pH values were traced during the experimental set-up, showing that the BSA elution from the column was achieved due to the pH change during the gradient elution.

The sample elution was fractionated and obtained fractions were evaluated for the amount of the eluted BSA.

The analytical evaluation of collected fractions is displayed in the Table 6, showing the amount of BSA bound per ml CV of the COSAN-cluster modified material.

TABLE 6

| | | COO— | | | |
|---|---|---|---|---|---|
| | | group | | | |
| | | density of | | | |
| | | the starting | | | |
| | Prepared | material | Amount of bound protein (mg/ml) | | |
| Material | as | (μeq/g) | Run 1 | Run 2 | Run 3 |
| TaKn-IV- | 1.8.11 | 1000 | 9.09 | 4.17 | 3.41 |
| 307 | | | | | |

Boron cluster modified material showed binding capacity for BSA ranging from 3-9 mg/ml. Moreover, this range was maintained in the consequential implementation of the material for at least 3 runs.

### REFERENCES

Bakardjiev, M.; Anwar, S. E.; Bavol, D.; Růžičková, Z.; Grüner, B. Focus on Chemistry of the 10-Dioxane-nido-7,8-dicarba-undecahydrido Undecaborate Zwitterion; Exceptionally Easy Abstraction of Hydrogen Bridge and Double-Action Pathways Observed in Ring Cleavage Reactions with OH⁻ as Nucleophile. Molecules 2020, 25, 814.
Bayguzina, A. R.; Lutfullina, A. R.; Khusnutdinov, R. I. Synthesis of N-(Adamantan-1-yl) carbamides by Ritter Reaction from Adamantan-1-ol and Nitriles in the Presence of Cu-Catalysts. Russ. J. Org. Chem. 2018, 54, 1127-1133.
Beckett, M. A.; Brellochs, B.; Chizhevsky, I. T.; Damhus, T.; Hellwich, K.-H.; Kennedy, J. D.; Laitinen, R.; Powell, W. H.; Rabinovich, D.; Viñas, C.; Yerin, A. Nomenclature for boranes and related species (IUPAC Recommendations 2019). Pure Appl. Chem. 2020, 92, 355-381.
Goszczyński, T. M.; Fink, K.; Kowalski, K.; Leśnikowski, Z. J.; Boratyński, J. Interactions of Boron Clusters and their Derivatives with Serum Albumin. Sci. Rep. 2017, 7, 9800.
Graalfs, H.; Graft Copolymer for Cation-exchange Chromatography. Merck Patent GmbH, WO2008145270, 2008.
Grimes, R. N. Carboranes; 3 ed.; Academic Press/Elsevier Inc.: London, U K, 2016
Ishii, S.; Nakamura, H. Synthesis and biological evaluation of closo-dodecaborate ibuprofen conjugate (DIC) as a new boron agent for neutron capture therapy. J. Organomet. Chem. 2018, 865, 178-182.
Kubasov, A. S.; Matveev, E. Y.; Retivov, V. M.; Akimov, S. S.; Razgonyaeva, G. A.; Polyakova, I. N.; Votinova, N. A.; Zhizhin, K. Y.; Kuznetsov, N. T. Nickel (II) complexes with nitrogen-containing derivatives of the closo-decaborate anion. Russ. Chem. Bull. 2014, 63, 187-193.
Kvasničková, E.; Masák, J.; Čejka, J.; Maťátková, O.; Šícha, V. Preparation, characterization, and the selective antimicrobial activity of N-alkylammonium 8-diethyleneglycol cobalt bis-dicarbollide derivatives. J. Organomet. Chem. 2017, 827, 23-31.
Plešek, J. Potential Applications of the Boron Cluster Compounds. Chem. Rev. 1992, 92, 269-278.
Řezáčová, P.; Pokorná, J.; Brynda, J.; Kožíšek, M.; Cígler, P.; Lepšík, M.; Fanfrlík, J.; Řezáč, J.; Grantz Šašková, K.; Sieglová, I.; Plešek, J.; Sícha, V.; Grüner, B.; Oberwinkler, H.; Sedláček, J.; Kräusslich, H.-G.; Hobza, P.; Král, P.; Konvalinka, J. Design of HIV Protease Inhibitors Based on Inorganic Polyhedral Metallacarboranes. J. Med. Chem. 2009, 52, 7132-7141.
Semioshkin, A.; Nizhnik, E.; Godovikov, I.; Starikova, Z.; Bregadze, V. I. Reactions of oxonium derivatives of [B₁₂H₁₂]²⁻ with amines: Synthesis and structure of novel B₁₂-based ammonium salts and amino acid. J. Organomet. Chem. 2007, 692, 4020-4028.
Sivaev, I. B.; Semioshkin, A. A.; Brellochs, B.; Sjöberg, S.; Bregadze, V. I. Synthesis of oxonium derivatives of the dodecahydro-closo-dodecaborate anion [B12H₁₂]²⁻. Tetramethylene oxonium derivative of [B₁₂H₁₂]²⁻ as a convenient precursor for the synthesis of functional compounds for boron neutron capture therapy. Polyhedron 2000, 19, 627-632.
Sokolov, V. B.; Aksinenko, A. Y.; Epishina, T. A.; Goreva, T. V.; Bachurin, S. O. Synthetic approaches to conjugation of aminoadamantanes and carbazoles. Russ. Chem. Bull. 2017, 66, 2110-2114.

1. Stationary phase comprising a base material and at least one boron cluster, wherein the boron cluster contains only main group elements of the periodic table.

2. Stationary phase according to claim 1, wherein the boron cluster has a cluster type selected from the group consisting of closo, nido, arachno, hypho, hypercloso, conjuncto, preferably closo, nido or conjuncto.

3. Stationary phase according to claim 1, wherein the boron cluster contains at least one atom that is not a boron atom.

4. Stationary phase according to claim 1, wherein one or more hydrogen atoms of the boron cluster are replaced by a covalently bonded substituent.

5. Stationary phase according to claim 1, wherein the at least one boron cluster is non-covalently or covalently bonded to the base material.

6. Stationary phase according to claim 1, wherein the at least one boron cluster is bonded to the base material via a linker.

7. Stationary phase according to claim 1, comprising polymer chains grafted onto the base material, whereby the polymer chains comprise at least one boron cluster.

8. Stationary phase according to claim 1, wherein the base material is a bead or a membrane.

9. Separation device comprising the stationary phase according to claim 1, preferably the separation device is a chromatography column.

10. A method for separation and/or purification of a target molecule, by the use of a boron cluster, wherein the boron cluster contains only main group elements.

11. Process for a separation and/or purification of a target molecule using a stationary phase according to claim 1.

12. Process for the separation of a target molecule from at least one other compound whereby a stationary phase according to claim 1 is contacted with a liquid comprising the target molecule and the at least one other compound and whereby the target molecule shows an interaction with the stationary phase that is different from the interaction of the other compound.

13. Process according to claim 12, whereby the stationary phase is present in a chromatography column and the liquid comprising the target molecule and the at least one other compound is run through the column whereby the target molecule and the other compounds present in the liquid are eluted from the column depending on their interaction with the stationary phase.

14. Process according to claim 12, whereby the process is performed by loading the stationary phase with an aqueous loading buffer of a certain pH and a certain ionic strength and eluting the target molecule with an aqueous elution buffer of another pH and/or another ionic strength, optionally with a further additive.

15. The process according to claim 11, whereby the target molecule is a protein.
